SEQUENCE LISTING (52 sequences)

SEQ ID NO: 1 - DNA, 1871 bp, Oryza sativa
atggcgtcctcgacgggggggttggaccacgggttcacgttcacgccgccgccgttcatcacgtcgttcaccgagctgctgtcggggggtggtggggacctgctcggcgccggcggtgaggagcgctcgccgagggggttctccagaggcggagcgagggtgggcggcggggtgcccaagttcaagtccgcgcagccgccgagcctgccgctctcgccgccgccggtgtcgccgtcgtcctacttcgccatcccgccggggctcagccccaccgagctgctcgactcccccgtcctcctcagctcctcccatgtgcgttccgcgtgacacaccttcatcttcttcttctacctttagtgcgcaattgctgtgtagtgtggtgctgatgatgatccatggcgtttgtgcgtgtttgtgcttttcagatcttggcgtccccgaccaccggtgcaatcccggctcagaggtacgactggaaggccagcgccgatctcatcgcttctcagcaagatgacagccgcggcgacttctccttccacaccaactccgacgccatggccgcgcaaccggcctctttcccttccttcaaggtacgtacaaatgcttcagctcatctagctcaacccaagctgaataaactgttgacagtctagctcaccatggttcatgtacgcttgtgctggagcaggagcaagagcagcaagtggtcgagtcgagcaagaacggcggcgccggcgcgtcgagcaacaagagcggcggcggcgggaacaacaagctggaggacgggtacaactggaggaagtacgggcagaagcaggtgaaggggagcgagaacccgaggagctactacaagtgcacctacaacggctgctccatgaagaagaaggtggagcgctcgctcgccgacggccgcatcacccagatcgtctacaagggcgcacacaaccaccccaagccgctctccacccgccgcaacgcctcctcctgcgccaccgccgccgcctgcgccgacgacctcgcggcgcccggcgcgggcgcggaccagtactccgccgcgacgcccgagaactcctccgtcacgttcggcgacgacgaggccgacaacgcatcgcaccgcagcgagggcgacgagcccgaagccaagcgctggtaagcctcacatcatcactcaaatcagcaatcgaagcgtaatatctccatgcgtgtgcgtgtgttcttcttgggtctcatggatgtcatggttttcaggaaggaggatgctgacaacgagggcagctccggcggcatgggcggcggcgccggcggcaagccggtgcgcgagccgaggcttgtggtgcagacgctgagcgacatcgacatcctcgacgacggcttccggtggaggaagtacggccagaaggtcgtcaagggcaaccccaacccaaggtaatcaatcaatcaagctgatcaaaatccgtcgattagtcgattaatcatatctgttcttgcgtctctaataatcaaatctgttcttgaatcgcaggagctactacaagtgcacgacggtgggctgcccggtgcggaagcacgtggagcgggcgtcgcacgacacgcgcgccgtgatcaccacctacgagggcaagcacaaccacgacgtcccggtcggccgcggcggcggcggcggacgcgccccggcgccggcgccgccgacgtcgggggcgatccggccgtcggccgtcgccgccgcccagcaggggccctacaccctcgagatgctccccaaccccgccggcctctacggcggctacggcgccggcgccggcggcgccgcgttcccgcgcaccaaggacgagcggcgggacgacctgttcgtcgagtcgctcctctgctag

SEQ ID NO: 2 - protein, 487 aa, Oryza sativa
MASSTGGLDHGFTFTPPPFITSFTELLSGGGGDLLGAGGEERSPRGFSRGGARVGGGVPKFKSAQPPSLPLSPPPVSPSSYFAIPPGLSPTELLDSPVLLSSSHILASPTTGAIPAQRYDWKASADLIASQQDDSRGDFSFHTNSDAMAAQPASFPSFKEQEQQVVESSKNGAAAASSNKSGGGGNNKLEDGYNWRKYGQKQVKGSENPRSYYKCTYNGCSMKKKVERSLADGRITQIVYKGAHNHPKPLSTRRNASSCATAAACADDLAAPGAGADQYSAATPENSSVTFGDDEADNASHRSEGDEPEAKRWKEDADNEGSSGGMGGGAGGKPVREPRLVVQTLSDIDILDDGFRWRKYGQKVVKGNPNPRSYYKCTTVGCPVRKHVERASHDTRAVITTYEGKHNHDVPVGRGGGGGRAPAPAPPTSGAIRPSAVAAAQQGPYTLEMLPNPAGLYGGYGAGAGGAAFPRTKDERRDDLFVESLLC

SEQ ID NO: 3 - DNA, 2175 bp, Oryza sativa
atgacaacctcgtcgtccgggagcgtcgagacgtcggccaattcgcggctggggacgttctcgttcgccagcgcgagcttcacggacctcctcgggggaaacgcgggagccggcggcggaggtgtgtccaggtacaaggccatgaccccgccttcgctgccgctctcgccgccgccggtgtcgccgtcgtccttcttcaacagcccgatcggcatgaaccaggccgacttcctcggctcgccggtgctcctgacctccagtgtaagcacgcacgcgaatgggccgagctccattaatggcctaatgccatgtcatttctgtacggtttttcagatgataatgagaaattgagaattgaaccttgccggttttcagatcttcccgtcgccgacgacgggcgcattcgcgtcgcagcactttgactggaggccggaggtggcggcggcgcagagcgccgatcaaggcggcaaggacgagcagaggaattcctactccgacttctcgtttcagacggcgcctgcgagcgaggaggccgtgcgaacgacgactttccagccaccggtcccaccggccccactggtgagtgagagcaaccataacgtcagctgttaccctttttggaggggtccttcgctgactttgtccaaaagaaactactcctagcatctttctttcttactcctagctaaggcattttcactatttcagtttgcaaattgcaatagtcaaaacccaaactatagaagtagcctctaaacaataaccttttctgacagggggacgaggcatacagaagtcagcagcagcagcagccatggggctaccagcagcagcctgcaggcatggacgcgggtgccaacgcggcgagcttcggtgcggcgccgttccaggcgacctcgtcggagatggcgccacaggtgcagggtggcggcgggtacagccagccgcagtcgcagaggcggtcgtcggacgacggctacaactggcgcaagtacgggcagaagcaggtgaaggggagcgagaacccccgcagctactacaagtgcaccttcccgaactgcccgaccaagaagaaggtggagcggtcgctcgacggccagatcaccgagatcgtgtacaagggcacgcacaaccacgccaagccgcagaacacgcgcaggaactccggctcgtcggcggcgcaggtcctgcagagcggcggcgacatgtcggagcattccttcgggggcatgtccggcacagcggcgacgcccgagaactcgtcggcgtcgttcggtgacgacgagatcagagttggctcccctcgggccgggaacggcggcggcgacgagttcgacgacgacgagccggattccaagagatggtgagtgccctgttcccaaacctcttcgcgccaaattatgtttgttgagataaaacagtgaactgaacatgttcaccataatatcaggaggaaagacggtgacggcgaggggatctccatggctggcaaccggacggtgcgggaaccgagggtggtcgtccaaaccatgagcgacatcgacatcctcgacgacggctaccgctggagaaagtacggccagaaggtggtgaagggcaacccgaacccaaggtaaataaagaacccaaaaagtccatttttcagttcgtctactggtgtgatcgcgcgtgtgtatctctcgtacttgtacttatcgtgcgtgctgctttcgtgtctgcaggagctactacaagtgcaccacggccggttgccccgtgcggaagcacgtggagcgcgcgtcccacgacctgcgcgccgtgatcaccacctacgaggggaaacacaaccacgacgtgcccgccgcgcgggggagcgccgcgctctaccgccccgcgccgccggcggcggccgccaccagcagccacccgtacctgccgaaccagccgccgcccatgtcctaccagcccacggggccacagccgtacgcgctgaggcccgacgggttcggcggccagggaccgttcggcggcgtggtcggcgggagcagcttcggcggcttctccgggttcgacgacgccaggggctcgtacatgagccagcaccagcagcagcagaggcagaacgacgcgatgcacgcctcgagagccaaggaagagcccggagacgacatgttcttccagaactcgctctactga

SEQ ID NO: 4 - protein, 555 aa, Oryza sativa
MTTSSSGSVETSANSRLGTFSFASASFTDLLGGNAGAGGGGVSRYKAMTPPSLPLSPPPVSPSSFFNSPIGMNQADFLGSPVLLTSSIFPSPTTGAFASQHFDWRPEVAAAQSADQGGKDEQRNSYSDFSFQTAPASEEAVRTTTFQPPVPPAPLGDEAYRSQQQQQPWGYQQQPAGMDAGANAASFGAAPFQATSSEMAPQVQGGGGYSQPQSQRRSSDDGYNWRKYGQKQVKGSENPRSYYKCTFPNCPTKKKVERSLDGQITEIVYKGTHNHAKPQNTRRNSGSSAAQVLQSGGDMSEHSFGGMSGTAATPENSSASFGDDEIRVGSPRAGNGGGDEFDDDEPDSKRWRKDGDGEGISMAGNRTVREPRVVVQTMSDIDILDDGYRWRKYGQKVVKGNPNPRSYYKCTTAGCPVRKHVERASHDLRAVITTYEGKHNHDVPAARGSAALYRPAPPAAAATSSHPYLPNQPPPMSYQPTGPQPYALRPDGFGGQGPFGGVVGGSSFGGFSGFDDARGSYMSQHQQQQRQNDAMHASRAKEEPGDDMFFQNSLY

SEQ ID NO: 5 - DNA, 2422 bp, Oryza sativa
atgaccgccgcgccggggagcctcccgctggtgaactcgaggcccgtctccctctccttggcggcgagcaggtcgtccttctccagcctgctcagtggcggcgccggctcgtcgttgaacctcatgacgccgccgtcttctctcccgccgtcgtcgccgtcgtcctacttcggcggcgtctcgtcctccgggtttctcgactcgccgatcctcctcacgcccagtgtaagcaagcacggccgccgtagccgatcgagcatcccatttcctttgccaaattgcaccggcatgtgtggctgattattgagctccatcgtgtttgtttcagttattcccatcgccgacgacgacgggcgcattgttcagctggattacgacggcgacggcgacggcggcgatagcgccggagagccaggtgcaaggaggggtcaaggacgagcagcaacagtactcggacttcacgttcctgccgacggcgtccacggcgccggcgacgacgatggccggagccaccgcgacgacgtccaactccttcatgcaggactccatgctaatggctccattggtaagacgaacatcaagctcaacttctaaattattagtgcagagctaagaaattacgaacagggactgacatgtgggccagtagtcgcatggtagtgagtgatttatcacaaacttttttactaaaaaaaagtaatgataagcttatccggatcaacgacgtccttgcattattacgaatttcttgtggattgggaggttctcttttgacttttatgccaaagcatacatatatgatgaaatgcattaatatctcgcggccaagatatcttaatcaacatgtttttttaatgataagggacactcaggctagatttgcaaaagcaaacttcattttcacgttctccatgaaaacctaagcacataccattagtcaagcttgctacagttctaaactagatcaattagtcaaaactcccttggaaaaaataattaccctgcatgtgatgcactccaaaaaaaagatgcatgtatcattgatattgttgtgtttgcaatattgtagggaggggacccgtacaatggcgagcagcagcagccatggagctaccaagaaccgaccatggacgctgacactaggccagcggaattcacctcgtcggcggcggcgggtgacgtggccgggaacggcagctacagccaggtggcggcgccggcggcggccggcggcttccgtcagcagagccggcggtcgtcggacgacggctacaactggcgcaagtacgggcagaagcagatgaaggggagcgagaacccgcgcagctactacaagtgcaccttccctggctgcccgaccaagaagaaggtggagcagtcgccggacggccaggtcaccgagatcgtctacaagggcgcgcacagccaccccaagccgccgcagaacggccgcggccgcggcggctccggctacgcgctgcatggcggcgccgccagcgacgcatactcctccgccgacgcgctctccggcacgccggtggcgacgcccgagaactcgtcggcgtcgttcggggacgacgaggcggtcaacggcgtcagctcgtcgctgcgggtcgcctctagcgtcggcggcggcgaggatctcgacgacgacgagcctgattccaagaggtggaggagagacggcggcgacggcgagggcgtctcgctggtggccggcaaccggacggtgcgtgagccgagggtggttgtgcagacgatgagcgacatcgacatcctcgacgacggctaccggtggcgcaagtacgggcagaaggtggtcaagggcaacccaaacccaaggtacgttgcatgcgtgcgtaaacatatatcgatctgtcacgtaggtgttcgacgcgtgtacgtgtgggctgacatgcatctgtgctctatctgcaggagctactacaagtgcacgacggccgggtgccccgtgcggaagcacgtggagcgcgcgtccaacgacctgcgcgcggtgatcaccacgtacgagggcaagcacaaccacgacgtgcccgcggcgcgcgggagcgccgccgccgcgctctaccgcgccacgccgccgccgcaggcgagcaacgccggcatgatgcccaccacggcgcagccctcgagctacctgcagggcggcggcggcgtccttccggccggcgggtacggcgcgtcgtacggcggcgcgccgacgacgacgcagcccgcgaacggcggtggcttcgccgccctgtccggccggttcgacgacgacgcgacgggagcgtcttactcttacacgagccagcagcagcagcagccgaacgacgcggtgtactacgcgtcgagagccaaggatgagccgagagacgacggcatcatgtcgttctttgagcagccgctgctgttttga

SEQ ID NO: 6 - protein, 572 aa, Oryza sativa
MTAAPGSLPLVNSRPVSLSLAASRSSFSSLLSGGAGSSLNLMTPPSSLPPSSPSSYFGGVSSSGFLDSPILLTPSLFPSPTTTGALFSWITTATATAAIAPESQVQGGVKDEQQQYSDFTFLPTASTAPATTMAGATATTSNSFMQDSMLMAPLGGDPYNGEQQQPWSYQEPTMDADTRPAEFTSSAAAGDVAGNGSYSQVAAPAAAGGFRQQSRRSSDDGYNWRKYGQKQMKGSENPRSYYKCTFPGCPTKKKVEQSPDGQVTEIVYKGAHSHPKPPQNGRGRGGSGYALHGGAASDAYSSADALSGTPVATPENSSASFGDDEAVNGVSSSLRVASSVGGGEDLDDDEPDSKRWRRDGGDGEGVSLVAGNRTVREPRVVVQTMSDIDILDDGYRWRKYGQKVVKGNPNPRSYYKCTTAGCPVRKHVERASNDLRAVITTYEGKHNHDVPAARGSAAAALYRATPPPQASNAGMMPTTAQPSSYLQGGGGVLPAGGYGASYGGAPTTTQPANGGGFAALSGRFDDDATGASYSYTSQQQQQPNDAVYYASRAKDEPRDDGIMSFFEQPLLF

SEQ ID NO: 7 - DNA, 1857 bp, Oryza sativa
atggccgattcgccaaaccctagctccggtgaccaccccgcgggcgtcggcgggtcgccggagaagcagcccccggtggatcggcgcgtcgcggcgctcgccgcgggcgcggcgggcgcgggggcgaggtacaaggcgatgtcccccgcgcggctgccgatctcgcgggagccctgcctcaccatccccgcgggcttcagcccctcggctctcctcgagtcccccgtcctcctcaccaacttcaaggttgaaccctctccgacaactggtactctgagcatggctgcaattatgaacaagagtgcaaatccagacatacttccttcgcctagggataaaacatctggtagcacccatgaagatggtggctctcgagattttgaattcaagcctcatctgaattcatcctctcaatcgacggcttctgctatcaatgatcccaaaaagcatgaaacttctatgaaaaatgaaagcctgaatactgccctgtcatctgacgatatgatgatcgacaatatacctctatgttctcgtgagtcaactctcgcagtcaatatttcaagtgccccgagccaactggttggaatggttggtttaactgacagctcacctgctgaagttggtacatctgagttgcatcagatgaatagctctggaaatgctatgcaggagtcacagcctgaaagtgtggctgaaaagtctgcagaggatggttataactggcgcaaatatgggcaaaagcatgttaagggaagtgagaacccgagaagctattacaagtgcacacatcctaactgtgatgtaaaaaagctattggagcgttcgcttgatggtcagattactgaagtggtttataaagggcgtcacaatcaccctaagccccaacccaataggaggctgtctgccggtgcagttcctccaatccagggtgaagaaagatatgatggtgtggcaactactgatgacaaatcttcaaatgttcttagcattcttggtaatgcagtacatacagctggtatgattgagcctgttccaggctcagctagtgatgatgacaatgatgccggaggagggagaccttaccctggagatgatgctgttgaggatgatgatttagagtcaaaacgaaggaaaatggaatctgctgctattgatgctgctttgatgggcaagcctaaccgtgagcctcgtgttgtagtacaaacggttagtgaggttgacatcttggatgatgggtaccgctggcgcaagtatggccagaaagtagttaaaggaaaccccaatccacggagttactacaagtgcacaaatacaggatgcccagtcaggaagcatgttgagagagcatcacatgatccaaaatcagtcataacaacatatgaaggaaaacataaccatgaagtccctgcgtcgaggaatgcgagccatgagatgtccactccccccatgaagcctgttgtccatccaattaacagcaatatgcagggccttggtggcatgatgagagcatgtgaacctaggacatttccaaaccaatattctcaagcagctgaaagtgacaccatcagccttgatcttggtgttggaatcagcccgaaccacagcgatgccacaaaccaattgcagtcctcagtttctgatcagatgcagtatcaaatgcagcccatgggttcagtatacagtaatatgggacttccagcaatggcaatgccgactatggctggcaatgcagctagcaatatatatggttcgagagaagaaaaacctagtgaaggttttactttcaaagccacaccgatggaccattcggctaacttatgctacagtaccgccggcaatttagtcatgggtccgtga

SEQ ID NO: 8 - protein, 618 aa, Oryza sativa
MADSPNPSSGDHPAGVGGSPEKQPPVDRRVAALAAGAAGAGARYKAMSPARLPISREPCLTIPAGFSPSALLESPVLLTNFKVEPSPTTGTLSMAAIMNKSANPDILPSPRDKTSGSTHEDGGSRDFEFKPHLNSSSQSTASAINDPKKHETSMKNESLNTALSSDDMMIDNIPLCSRESTLAVNISSAPSQLVGMVGLTDSSPAEVGTSELHQMNSSGNAMQESQPESVAEKSAEDGYNWRKYGQKHVKGSENPRSYYKCTHPNCDVKKLLERSLDGQITEVVYKGRHNHPKPQPNRRLSAGAVPPIQGEERYDGVATTDDKSSNVLSILGNAVHTAGMIEPVPGSASDDDNDAGGGRPYPGDDAVEDDDLESKRRKMESAAIDAALMGKPNREPRVVVQTVSEVDILDDGYRWRKYGQKVVKGNPNPRSYYKCTNTGCPVRKHVERASHDPKSVITTYEGKHNHEVPASRNASHEMSTPPMKPVVHPINSNMQGLGGMMRACEPRTFPNQYSQAAESDTISLDLGVGISPNHSDATNQLQSSVSDQMQYQMQPMGSVYSNMGLPAMAMPTMAGNAASNIYGSREEKPSEGFTFKATPMDHSANLCYSTAGNLVMGP

SEQ ID NO: 9 - DNA, 2025 bp, Oryza sativa
atggacgggaccaacaaccatggagcactgatggacgattggatgcttccctcacccagtccaagaacactcatgtcgagtttcttgaacgaagaattcagctccggtcccttttcagacattttctgtgataatggcagtaacaaacatcaggatggacttgggaagagcaaagctttcatcgattcaagccgggaagaaactgctcagctagcaaaaaagtttgaatcaaacctttttggtgccaaccagaaatcaagctcaaatggctgtctgtcagagaggatggctgcaaggacaggttttggtgtcctgaaaattgatacatctcgtgtcggttattctacaccgattcggtctccggtgacgatcccgcccggtgtgagtccaagggaacttcttgagtcgccggtttttcttccgaacgccattgcacaaccttctcctaccactggcaaactgccatttttgatgcatagtaatgttaaaccatcgatccctaaaaaaactgaagatgaaacacgccatgatcgtgtattcttctttcaacccattttgggatctaagccaccaacttgtccagttgcagagaagggtttcagtgttaatcatcaaaaccagccttcagtgacggataatcaccaggagctcagtcttcagtctagctcaactgcagccaaggatttcacttcagcaactattgttaaacctaagacatctgattccatgttagacaatgatgatcacccttcccctgcaaatgatcaagaagagaatgcaacaaacaaaaatgaagagtattcttcagacctgatcattacccctgctgaggatggatataactggaggaaatatggacagaagcaagttaagaacagtgagcatcccagaagctactacaaatgcactttcacgaattgcgctgtcaagaaggtggagcgttctcaagatggccaaataacagagatagtctacaaaggttctcacaatcaccctttgccgccttccaaccgccgaccaaatgttcctttctcacacttcaatgatctgagagatgatcactctgagaaatttggttccaagtctggtcaggccacagcaacttcatgggagaatgccgcaaatggacacctccaagatgtcggtagtgaagttctgacaaaactgtctgcttctcttacgacaacagaacatgctgaaaaatctgttatggacaaacaagaagctgtggatatctcatcgacgctctccaatgaagaggatgatagggtaacgcatcgtgccccgctttctctgggctttgatgcgaacgatgactatgttgaacacaagagaagaaagatggatgtttatgccgctactagcactagcaccaacgccatcgacataggagctgtggcgtcaagagctatccgggagcctcgcgttgttgttcagaccacaagtgaggttgacatccttgatgatggttaccgttggcgcaagtatgggcagaaagttgtcaaaggaaacccaaatccaaggagctactacaagtgcactcatccgggttgctcggtgcgcaagcatgtggagcgatcatcgcatgatctgaaatccgtcatcacgacgtatgaaggaaagcacaaccatgaagttccagctgccaggaacagtggccacccaagctcaggctcagccgctgcaccacaggctaccaatggtcttcttcaccggagacctgaaccggcacaaggtggtggtggtggtagccttgctcagtttggctatggctcagctggtcacagaccagcagagcagtttggtgcagcagcagctggtttctcctttggaatgctgcctcgtagcattgcaactccggcgccgtctccggcgatcgccgtgccggcgatgcaggggtacccagggcttgtgctgccgagaggtgagatgaaggtgaacttgctgccacagtctgggaatgctggtgcagcagctagccagcagctgatgggcaggttgccaaagcagcatcctcagatgtaa

SEQ ID NO: 10 - protein, 674 aa, Oryza sativa
MDGTNNHGALMDDWMLPSPSPRTLMSSFLNEEFSSGPFSDIFCDNGSNKHQDGLGKSKAFIDSSREETAQLAKKFESNLFGANQKSSSNGCLSERMAARTGFGVLKIDTSRVGYSTPIRSPVTIPPGVSPRELLESPVFLPNAIAQPSPTTGKLPFLMHSNVKPSIPKKTEDETRHDRVFFFQPILGSKPPTCPVAEKGFSVNHQNQPSVTDNHQELSLQSSSTAAKDFTSATIVKPKTSDSMLDNDDHPSPANDQEENATNKNEEYSSDLIITPAEDGYNWRKYGQKQVKNSEHPRSYYKCTFTNCAVKKVERSQDGQITEIVYKGSHNHPLPPSNRRPNVPFSHFNDLRDDHSEKFGSKSGQATATSWENAANGHLQDVGSEVLTKLSASLTTTEHAEKSVMDKQEAVDISSTLSNEEDDRVTHRAPLSLGFDANDDYVEHKRRKMDVYAATSTSTNAIDIGAVASRAIREPRVVVQTTSEVDILDDGYRWRKYGQKVVKGNPNPRSYYKCTHPGCSVRKHVERSSHDLKSVITTYEGKHNHEVPAARNSGHPSSGSAAAPQATNGLLHRRPEPAQGGGGGSLAQFGYGSAGHRPAEQFGAAAAGFSFGMLPRSIATPAPSPAIAVPAMQGYPGLVLPRGEMKVNLLPQSGNAGAAASQQLMGRLPKQHPQM

SEQ ID NO: 11 - DNA, 2334 bp, Oryza sativa
atggaggagtggaaggattccaaccatcgaggcgcggattacctgatgacgatgccgatgcagaacttcctcgccgacgcgttcccgccaccggagctcttggagggagaaggcgggttcgagaagcacggcctgtcggtggccgttggctcgccgccgccgacgccgccgcctccggaggacgggtgctcgccgctgccactgacgccgcagttcggccagaagttcggctccggcggcggcggcggcggcagcctcgccgacaggagggcgagaggcgggttcagcaacgtcgccaggatcagcgtgccgtacaaccagccggcggcggacgtgtcgtcggcgggggcgccgtcgccgtacgtgacgatcccgcccggcctgagcccgacgactctgctggaatcgccggtcttctccaatgccatgggccaggcctcgccgaccactgggaagctgcacatgcttggtggtgccaacgacagcaatccaatcagatttgaatcccctcggatcgaagaaggatctggtgcattttctttcaagcctctgaatctcgcatcctcacactacgcagctgaagaaaagacgaaatctctacccaacaaccagcatcagtcgctaccgatttctgtcaagactgaagctactagcattcaaaccgcacaagatgaagcagcagccaaccaactgatgcagccgcagttcaacggcggcaagcggagccgcgctgcacctgacaacggcggcgacggcgagggccagccggcggagggcgacgcgaaggctgactcctcctccggcgcggccgcggtcgccgtcgtcgccgccgccgcggcggcggtggcggaggacgggtacagctggaggaagtacgggcagaagcaggtgaagcacagcgagtacccgaggagctactacaagtgcacgcacgccagctgcgcggtgaagaagaaggtggagcgctcgcacgagggccacgtcacggagatcatctacaagggcacccacaaccaccccaagccggcggcgagccgccgcccccccgtccatcctccgccgccgtcgccggcgacgacgacgacgacgccgctgccgccaggcgacgcgcaggccgaccacgcgcccgacggcggcggcggcagtaccccagttggcgccggacaggcgggcgcggagtggcacaacggcggcgtggttggcggcgaggggctggtggacgcgacgtcgtctccctccgtccccggcgagctctgcgagtcgacggcgtcgatgcaggtccatgaaggcgcggcggcggcgcagctgggggaatcccccgagggcgtcgacgtcacgtctgcggtgtccgacgaggtggacagggatgacaaggcgacgcacgtgttgcccctggccgccgccgccgccgacggcgagagcgacgagctggagcgaaagagaaggaagctggactcctgcgccaccatggacatgagcacggcgtcgagggcggtgcgcgagccgcgggtggtgatccagacgacgagcgaggtggacatcctcgacgacggctaccgctggcgcaagtacgggcagaaggtcgtcaaggggaaccccaacccaagctcctcctcctccatggatgctgatcgatctctcgtcgtcgtcgtcgtgatcaggagctactacaagtgcacgcacccggggtgcctggtgcggaagcacgtggagcgcgcgtcgcacgacctcaagtcggtgatcaccacgtacgaggggaagcacaaccacgaggtccccgcggcgaggaacagcggccacccggcgggctcggcttcgcccggcggcggcgcggggtcgtcgtcgcagccccacggcgtcggcgtcggcgggcgcaggccggaggtgccgtcggtgcaggagagcctgatgaggctcggcggcggctgcggcgcggcgccgttcccgccccacttcggcctgcacctgccgccgccgccgccgagggacccgctcgcgccgatgagcaacttcccctactcgctcggccacgcgccgtcgccggcgctgcggggcctgccgccgcccccgcccccgccgccgtcggcgtcggcgctggcggtggcggggctcggcggcgtggtggaggggctcaagtacccgatgctggcgccgccgtcggtgcactcgctgctgaggcaccgccagggcggcggcatggaggcggtggtggtccccaaggcggaggtgaagcaggaggcgatgcggcccgccgccgccgtcgccggcgcggggcgcggcgcggcggtgtatcagcaggcgatgagcagggtgtcgctggggaatcaactgtag

SEQ ID NO: 12 - protein, 776 aa, Oryza sativa
MEEWKDSNHRGADYLMTMPMQNFLADAFPPPELLEGEGGFEKHGLSVAVGSPPPTPPPPEDGCSPLPLTPQFGQKFGSGGGGGGSLADRRARGGFSNVARISVPYNQPAADVSSAGAPSPYVTIPPGLSPTTLLESPVFSNAMGQASPTTGKLHMLGGANDSNPIRFESPRIEEGSGAFSFKPLNLASSHYAAEEKTKSLPNNQHQSLPISVKTEATSIQTAQDEAAANQLMQPQFNGGKRSRAAPDNGGDGEGQPAEGDAKADSSSGAAAVAVVAAAAAAVAEDGYSWRKYGQKQVKHSEYPRSYYKCTHASCAVKKKVERSHEGHVTEIIYKGTHNHPKPAASRRPPVHPPPPSPATTTTTPLPPGDAQADHAPDGGGGSTPVGAGQAGAEWHNGGVVGGEGLVDATSSPSVPGELCESTASMQVHEGAAAAQLGESPEGVDVTSAVSDEVDRDDKATHVLPLAAAAADGESDELERKRRKLDSCATMDMSTASRAVREPRVVIQTTSEVDILDDGYRWRKYGQKVVKGNPNPSSSSSMDADRSLVVVVVIRSYYKCTHPGCLVRKHVERASHDLKSVITTYEGKHNHEVPAARNSGHPAGSASPGGGAGSSSQPHGVGVGGRRPEVPSVQESLMRLGGGCGAAPFPPHFGLHLPPPPPRDPLAPMSNFPYSLGHAPSPALRGLPPPPPPPPSASALAVAGLGGVVEGLKYPMLAPPSVHSLLRHRQGGGMEAVVVPKAEVKQEAMRPAAAVAGAGRGAAVYQQAMSRVSLGNQ

SEQ ID NO: 13 - DNA, 1182 bp, Arabidopsis thaliana
atgtcttccacttctttcaccgaccttcttggttcttccggcgttgactgttacgaagatgatgaagacttgagagtttctgggtcgagttttggtgggtactatccagagagaaccgggtctggtttacctaagttcaagacggctcaaccaccacctcttccgatttcacaatcttctcataacttcactttctccgattaccttgattctcctctgcttctcagctcctcacacagtttgatatctccaacaacaggaacgtttccattgcaaggctttaatggaacaacaaacaatcactcagattttccctggcagctacaatctcaaccatcaaacgcttcttctgctttgcaagaaacatatggtgttcaagatcacgagaagaagcaggagatgattcctaatgagattgcaacacaaaacaacaatcaaagttttggaacagaacgtcagataaagataccagcatacatggtgagtaggaactctaatgatggttatggttggagaaaatacggtcagaaacaagtgaagaagagcgaaaaccctaggagttacttcaagtgtacgtatcctgattgtgtttccaagaagattgttgagacggcttctgatggacagatcactgagatcatttataaaggtggtcataatcatcctaagcctgagttcaccaagagaccatctcaatcttcattaccatcatcggttaatgggaggcgcttgtttaatcctgcttctgttgttagtgaacctcatgatcaatcagagaactcttcgatttcgtttgactatagtgatcttgagcagaaaagttttaaatcagagtatggtgagatagatgaagaggaggaacaacctgagatgaagaggatgaaaagagaaggtgaagatgaagggatgtctatagaagtaagcaaaggagttaaagagccaagagttgtggttcagacaataagtgatattgatgttcttatagatggctttagatggaggaaatatggtcaaaaagttgtcaaaggaaatactaatccaaggagctactacaagtgcacattccaaggttgtggagtgaagaagcaagtggaaagatccgcagcagacgagagagcagttctcactacctatgaaggaagacacaatcacgatatcccaaccgcgctacgtcgctcgtga

SEQ ID NO: 14 - protein, 393 aa, Arabidopsis thaliana
MSSTSFTDLLGSSGVDCYEDDEDLRVSGSSFGGYYPERTGSGLPKFKTAQPPPLPISQSSHNFTFSDYLDSPLLLSSSHSLISPTTGTFPLQGFNGTTNNHSDFPWQLQSQPSNASSALQETYGVQDHEKKQEMIPNEIATQNNNQSFGTERQIKIPAYMVSRNSNDGYGWRKYGQKQVKKSENPRSYFKCTYPDCVSKKIVETASDGQITEIIYKGGHNHPKPEFTKRPSQSSLPSSVNGRRLFNPASVVSEPHDQSENSSISFDYSDLEQKSFKSEYGEIDEEEEQPEMKRMKREGEDEGMSIEVSKGVKEPRVVVQTISDIDVLIDGFRWRKYGQKVVKGNTNPRSYYKCTFQGCGVKKQVERSAADERAVLTTYEGRHNHDIPTALRRS

SEQ ID NO: 15 - DNA, 941 bp, Arabidopsis thaliana
atgggctcttttgatcgccaaagagctgttccgaaattcaaaacagcaacaccgtcaccgctccctctttctccttcgccttacttcactatgcctcctggccttactcccgccgactttctcgactctcctcttctcttcacttcctccaacattttgccgtctcctacgacaggcacatttccagcgcaatctctgaactataacaataacggtttgctcattgacaaaaatgaaatcaaatatgaagacacaactcctcccttgttcctaccatctatggtaactcagcctttacctcaactggatttattcaaatccgaaatcatgtcgagtaacaaaacctctgatgacggctacaattggcgcaaatacgggcagaagcaagtcaaaggaagcgaaaacccgaggagttacttcaaatgcacgtatccaaattgtcccacaaagaagaaagtagagacgtctcttgtgaagggtcagatgattgagattgtctataaaggaagccacaatcatcccaagccccaatccacgaagcgatcatcttccaccgctatagcagcacatcagaacagcagtaatggagacggtaaagacattggtgaagatgaaacagaggccaagagatggaaaagagaagagaatgtgaaggagccaagagtggtggttcagacaacaagtgatatagacattcttgacgatggctacagatggagaaagtatggtcagaaagtcgtcaagggtaatccaaatccaaggagctattacaagtgcacatttacaggatgttttgtaaggaaacacgttgaaagagcatttcaagatcccaagtcagtgatcacaacttacgaaggaaaacacaaacaccaaatcccgaccccaagaagaggtccagttttaaggttacttggaaaaacagagacataaagagcaaatga

SEQ ID NO: 16 - protein, 309 aa, Arabidopsis thaliana
MGSFDRQRAVPKFKTATPSPLPLSPSPYFTMPPGLTPADFLDSPLLFTSSNILPSPTTGTFPAQSLNYNNNGLLIDKNEIKYEDTTPPLFLPSMVTQPLPQLDLFKSEIMSSNKTSDDGYNWRKYGQKQVKGSENPRSYFKCTYPNCPTKKKVETSLVKGQMIEIVYKGSHNHPKPQSTKRSSSTAIAAHQNSSNGDGKDIGEDETEAKRWKREENVKEPRVVVQTTSDIDILDDGYRWRKYGQKVVKGNPNPRSYYKCTFTGCFVRKHVERAFQDPKSVITTYEGKHKHQIPTPRRGPVLRLLGKTET

SEQ ID NO: 17 - DNA, 1560 bp, Arabidopsis thaliana
atggctgcttcttttcttacaatggacaatagcagaaccagacaaaacatgaatggttctgctaattggtcacaacaatccggaagaacatctacttcctctttggaagatcttgagataccaaagttcagatcttttgctccttcttcaatctctatctctccttctcttgtctctccttccacttgtttcagtccctctctttttctcgattcccctgcttttgtctcctcctctgctaacgttctagcttctccaaccacaggagctttaatcacaaacgtaactaaccagaaaggtataaatgaaggagacaagagcaacaacaacaactttaacttattcgatttctcattccacacacaatcatcaggagtttctgctccgaccacaactacaactacaactacaactacaacaacaacaaacagttctatctttcaatctcaggaacaacagaagaagaaccagtcagaacaatggagccaaaccgagactcgtccaaacaatcaagctgtatcttacaatggaagagagcaaaggaaaggagaggatggttacaattggagaaagtacggacaaaaacaggtgaaaggaagtgagaatcctcggagttactataagtgtactttccctaattgtccaacgaagaagaaagtggagagatctttggaaggtcagatcacagagattgtgtataaaggaagccacaaccatcctaaacctcagtctactagaagatcttcttcgtcttcttcgacttttcattcagctgtgtacaatgccagtttggatcataatcgtcaagcttcttctgatcagcctaattccaataatagctttcatcagtctgattcctttgggatgcaacaagaggataatactacttctgattctgttggtgacgatgagttcgaacaaggctcatcgattgtcagcagagacgaagaagattgtgggagtgaacctgaagcaaagagatggaaaggggacaatgaaacaaatggtgggaatggtggtggaagcaagacagtgagagagccgagaatcgtagtgcagacaacgagtgatattgacattcttgacgacggttacagatggagaaaatacggccagaaagtcgttaagggaaacccaaatccaagaagctactacaagtgcacaaccatcggttgtccagtgaggaaacatgtggagagagcatcacacgacatgagagcagtaatcacaacctacgaagggaaacacaaccacgatgttcctgcagctcgtggtagcggttacgccacaaacagagcaccacaggattcgtcttcagtcccgattagaccagctgctattgctggtcactccaactacactacttcttctcaagcaccatatacacttcagatgctgcacaacaacaacactaataccgggccttttggttacgccatgaacaacaataacaacaacagcaaccttcaaacgcaacaaaactttgttggtggtggattctctagagcaaaggaagaaccaaacgaggagacctcatttttcgattcgtttatgccctga

SEQ ID NO: 18 - protein, 519 aa, Arabidopsis thaliana
MAASFLTMDNSRTRQNMNGSANWSQQSGRTSTSSLEDLEIPKFRSFAPSSISISPSLVSPSTCFSPSLFLDSPAFVSSSANVLASPTTGALITNVTNQKGINEGDKSNNNNFNLFDFSFHTQSSGVSAPTTTTTTTTTTTTTNSSIFQSQEQQKKNQSEQWSQTETRPNNQAVSYNGREQRKGEDGYNWRKYGQKQVKGSENPRSYYKCTFPNCPTKKKVERSLEGQITEIVYKGSHNHPKPQSTRRSSSSSSTFHSAVYNASLDHNRQASSDQPNSNNSFHQSDSFGMQQEDNTTSDSVGDDEFEQGSSIVSRDEEDCGSEPEAKRWKGDNETNGGNGGGSKTVREPRIVVQTTSDIDILDDGYRWRKYGQKVVKGNPNPRSYYKCTTIGCPVRKHVERASHDMRAVITTYEGKHNHDVPAARGSGYATNRAPQDSSSVPIRPAAIAGHSNYTTSSQAPYTLQMLHNNNTNTGPFGYAMNNNNNNSNLQTQQNFVGGGFSRAKEEPNEETSFFDSFMP

SEQ ID NO: 19 - DNA, 2064 bp, Arabidopsis thaliana
atggctggttttgatgaaaatgttgctgtgatgggagaatgggtgcctcgtagtcctagtcccgggacacttttctcctctgctattggagaagagaagagctcgaaacgtgttcttgaaagagagttatctttgaatcatggtcaagttattggtttagaagaagacactagtagtaatcataacaaggattcttcacaaagcaatgtttttcgaggtggtctcagtgaaagaattgctgcaagagctggatttaatgctccaaggttgaacactgagaatatccgcaccaacaccgacttttccattgactctaaccttcgatctccttgcttaaccatctcttctcctggccttagccctgcaacactcttggaatctcctgttttcctttctaacccattggctcaaccttctccaactaccgggaaatttccatttcttcctggtgttaatggtaatgcattgtcttctgagaaagcgaaagacgagttctttgatgatattggagcatcattcagcttccatcctgtttcaagatcatcttcctctttcttccaaggcacaacagagatgatgtcagttgattatggtaactacaacaatagatcttcttctcatcaatccgcagaagaagtaaaacctggctctgaaaacatagaaagctccaatctttatgggattgaaactgacaatcaaaacgggcagaacaagacatctgatgtcactacaaacaccagtcttgaaaccgtggatcatcaagaggaagaagaagagcaaagacgcggtgattcgatggctggtggtgcgcctgcagaggatggatataactggaggaaatacggacaaaagttggtcaaaggaagtgagtatccgcgaagctattacaagtgcacaaacccgaattgtcaggtgaagaagaaagttgagagatcaagggaaggtcacatcacagagattatatacaaaggagctcataatcatcttaaacctccacctaatcgccgctcagggatgcaagtagatggaactgaacaagttgaacaacaacaacaacagagagattctgctgcaacgtgggttagttgtaataacactcaacaacaaggtggaagcaatgagaacaatgtcgaagagggatctacgagattcgagtatggaaaccaatctggatcaattcaagctcaaaccggaggtcaatacgagtcaggtgatcctgtggttgtggttgatgcttcttcaacattctctaatgatgaagatgaagatgatcgagggacacatggaagtgtttctttgggttacgatggaggaggaggaggtgggggaggagaaggagatgaatcagagtcgaaaagaaggaaactagaagcttttgcagcagagatgagtggatcaacaagagccatacgtgagccaagagttgttgtgcagacaacgagtgatgttgacattcttgatgatggttatcgctggcgaaaatatggtcagaaagttgtcaaaggcaatccaaatccaaggagttattacaaatgcacagctccaggatgtacagtgaggaaacatgttgaaagagcttctcatgatctcaaatccgttataacaacttacgaaggcaaacataaccatgacgtccccgctgcacgcaacagcagccacggaggcggtggtgatagtggtaacggtaacagcggcggttcagccgcagtttctcaccattaccacaacggtcatcactcagagccgccacgtgggagattcgacagacaagtcacaactaacaatcagtctccttttagccgtccctttagctttcagccacatttgggtcctccttctggtttctccttcggtttaggacaaaccggtttggttaatctttcaatgcctggtttagcgtatggtcaagggaaaatgccgggtttgcctcacccgtatatgacacaaccggttgggatgagtgaagcaatgatgcagagagggatggaaccaaaggttgaaccggtttcagattcaggacaatcggtatataaccagatcatgagtagattacctcagatttga

SEQ ID NO: 20 - protein, 687 aa, Arabidopsis thaliana
MAGFDENVAVMGEWVPRSPSPGTLFSSAIGEEKSSKRVLERELSLNHGQVIGLEEDTSSNHNKDSSQSNVFRGGLSERIAARAGFNAPRLNTENIRTNTDFSIDSNLRSPCLTISSPGLSPATLLESPVFLSNPLAQPSPTTGKFPFLPGVNGNALSSEKAKDEFFDDIGASFSFHPVSRSSSSFFQGTTEMMSVDYGNYNNRSSSHQSAEEVKPGSENIESSNLYGIETDNQNGQNKTSDVTTNTSLETVDHQEEEEEQRRGDSMAGGAPAEDGYNWRKYGQKLVKGSEYPRSYYKCTNPNCQVKKKVERSREGHITEIIYKGAHNHLKPPPNRRSGMQVDGTEQVEQQQQQRDSAATWVSCNNTQQQGGSNENNVEEGSTRFEYGNQSGSIQAQTGGQYESGDPVVVVDASSTFSNDEDEDDRGTHGSVSLGYDGGGGGGGGEGDESESKRRKLEAFAAEMSGSTRAIREPRVVVQTTSDVDILDDGYRWRKYGQKVVKGNPNPRSYYKCTAPGCTVRKHVERASHDLKSVITTYEGKHNHDVPAARNSSHGGGGDSGNGNSGGSAAVSHHYHNGHHSEPPRGRFDRQVTTNNQSPFSRPFSFQPHLGPPSGFSFGLGQTGLVNLSMPGLAYGQGKMPGLPHPYMTQPVGMSEAMMQRGMEPKVEPVSDSGQSVYNQIMSRLPQI

SEQ ID NO: 21 - DNA, 1707 bp, Arabidopsis thaliana
atggctggtattgataataaagctgctgtaatgggagaatggttcgactgtagtactactaaccacaggaagagatcgaaagcggaacttggtagagagttttctttaaattacatcaagaatgaggattctttgcaaaccacctttcaagaaagttcacgaggagctcttcgtgaaaggattgctgcgagatccgggtttaatgcaccgtggttaaacactgaggatattcttcagtcgaaatctttaaccatctcttctcctggtcttagtcctgcaactctgttagagtctcctgttttcctctcaaaccctttgctatctccaacaaccgggaagctctcatcagtaccttctgataaggctaaagctgagttatttgacgacattaccacatccttagccttccaaaccatttcaggaagtggccttgatcctactaacatcgctttagaacccgatgattcccaagactatgaagaaagacagctcggcggtttaggagactcgatggcttgttgtgcacctgcagatgatggatacaactggagaaaatatggacaaaagctagttaaaggaagtgagtatccgcggagctattacaagtgcacgcacccgaattgtgaggccaagaagaaggttgaacggtctcgggaaggtcatattatagagatcatatacacaggagatcatatacacagcaaacctccacctaaccgccggtcagggattggatcatccggtactggccaagacatgcaaatagatgcaaccgaatacgaaggttttgctggaaccaatgagaacatagaatggacatcacctgtatctgcagagctcgaatacggaagccattcaggatcaatgcaggttcaaaacgggactcatcagttcgggtatggtgatgcagcagctgatgccttatatagagatgaaaacgaagatgatcgcacgtcccacatgagtgtttccctgacttacgatggagaggtagaagagtccgaatcaaagagaaggaaactagaagcttatgcaacagaaacgagtggatcaaccagagccagccgtgagccaagagttgtggtgcagaccacaagtgacattgacatcctcgatgatggttatcgctggcgcaagtatgggcaaaaagtcgttaaaggaaacccgaatccaaggagctactataaatgcacagctaatggatgtaccgtaacgaagcatgtagagagagcctctgatgacttcaagagcgtactaacaacttatataggcaagcacacccacgttgtaccagcagcacgcaacagcagccacgtcggtgcaggcagttcagggactctccaaggcagtttagcgactcagacccacaaccacaatgtgcactatccaatgccacacagtagatctgagggactggccacagccaactcatctctatttgacttccagtcacacctgaggcatcctacaggtttctccgtttacataggccaatctgagctttctgatctttcaatgcctggtctaactattgggcaagagaagcttaccagcctgcaggcgcctgacattggggatccaactggcctaatgttgcagttagcagcacagccgaaggtggaaccagtgtcaccacaacagggacttgatttgtcagcgagctcattgatatgcagagagatgttgagtagattacgacagatatga

SEQ ID NO: 22 - protein, 568 aa, Arabidopsis thaliana
MAGIDNKAAVMGEWFDCSTTNHRKRSKAELGREFSLNYIKNEDSLQTTFQESSRGALRERIAARSGFNAPWLNTEDILQSKSLTISSPGLSPATLLESPVFLSNPLLSPTTGKLSSVPSDKAKAELFDDITTSLAFQTISGSGLDPTNIALEPDDSQDYEERQLGGLGDSMACCAPADDGYNWRKYGQKLVKGSEYPRSYYKCTHPNCEAKKKVERSREGHIIEIIYTGDHIHSKPPPNRRSGIGSSGTGQDMQIDATEYEGFAGTNENIEWTSPVSAELEYGSHSGSMQVQNGTHQFGYGDAAADALYRDENEDDRTSHMSVSLTYDGEVEESESKRRKLEAYATETSGSTRASREPRVVVQTTSDIDILDDGYRWRKYGQKVVKGNPNPRSYYKCTANGCTVTKHVERASDDFKSVLTTYIGKHTHVVPAARNSSHVGAGSSGTLQGSLATQTHNHNVHYPMPHSRSEGLATANSSLFDFQSHLRHPTGFSVYIGQSELSDLSMPGLTIGQEKLTSLQAPDIGDPTGLMLQLAAQPKVEPVSPQQGLDLSASSLICREMLSRLRQI

SEQ ID NO: 23 - DNA, 1674 bp, Arabidopsis thaliana
atgaaccctcaagctaatgaccggaaggagtttcagggagattgttcggcgacgggagatctcacggcaaagcacgattcagctggaggaaacggaggtggaggtgctaggtataagctgatgtcaccggccaagcttccgatctcgaggtcgactgatatcacgattcctcctgggttgagtccgacttcgtttttggaatctcctgttttcatctccaacatcaagccagaaccttcccctactactggttctttgttcaagcctcgaccagtgcacatttctgctagctcaagttcttatacaggcagggggttccatcagaacacctttactgagcagaagtccagtgaatttgagttcagacctcctgcatcaaatatggtatatgcagagcttggcaagattagaagtgagccaccagtacattttcaaggccagggccatggatcctcacactcaccttcttcgatcagtgatgctgcaggttcctcaagtgagctaagccggccaactcctccttgtcagatgacaccaacgagctcagatattccggctggatctgatcaagaggaatcaatccagacttcccaaaatgactccagaggaagcactccatccatcttggctgatgatggttataactggagaaaatatggtcaaaagcatgtcaaagggagtgaatttccccggagctattataaatgtacacatcctaattgtgaagtgaaaaagttatttgaaagatctcatgatgggcagatcaccgatattatatacaagggtacacatgaccatcctaaacctcaacctggtcgccgaaactctggtggtatggctgcacaagaagaaaggctagacaagtatccttcttcaactggccgagatgagaagggatctggcgtctacaacttgtctaaccccaatgaacaaactggtaaccctgaagtacctcctatctcagcatctgacgatggtggagaagcggcagcgtcaaataggaataaagatgagccggacgatgatgatccattctcaaaacggaggaggatggagggtgcgatggaaataactccactagtgaaacccatccgggagcctcgggttgttgttcaaactctgagtgaggttgacattctggatgatggttatagatggcgcaaatatgggcagaaagtcgtaagggggaacccaaatcccaggagctactacaaatgcacagctcatggatgcccagtgagaaaacacgtggagagagcatcacatgatccaaaagctgtaataacaacatacgaaggcaaacacgatcatgatgttcccacttcaaagtctagcagcaatcacgaaatccagcctcggttcagaccagatgaaacagacaccatcagcctcaatcttggtgttggaatctcatctgatggacctaaccacgcttccaacgaacatcagcaccagaatcaacaacttgtcaaccaaactcacccaaatggagtcaatttcaggtttgttcatgctagtcccatgtcatcctactatgctagcttaaatagcggtatgaatcagtacggccagagagaaacaaagaacgagactcaaaatggtgacatctcgtccttgaacaattcatcttacccatatccgcccaacatggggagagtacaatcgggtccgtaa

SEQ ID NO: 24 - protein, 556 aa, Arabidopsis thaliana
MNPQANDRKEFQGDCSATGDLTAKHDSAGGNGGGGARYKLMSPAKLPISRSTDITIPPGLSPTSFLESPVFISNIKPEPSPTTGSLFKPRPVHISASSSSYTGRGFHQNTFTEQKSSEFEFRPPASNMVYAELGKIRSEPPVHFQGQGHGSSHSPSSISDAAGSSSELSRPTPPCQMTPTSSDIPAGSDQEESIQTSQNDSRGSTPSILADDGYNWRKYGQKHVKGSEFPRSYYKCTHPNCEVKKLFERSHDGQITDIIYKGTHDHPKPQPGRRNSGGMAAQEERLDKYPSSTGRDEKGSGVYNLSNPNEQTGNPEVPPISASDDGGEAAASNRNKDEPDDDDPFSKRRRMEGAMEITPLVKPIREPRVVVQTLSEVDILDDGYRWRKYGQKVVRGNPNPRSYYKCTAHGCPVRKHVERASHDPKAVITTYEGKHDHDVPTSKSSSNHEIQPRFRPDETDTISLNLGVGISSDGPNHASNEHQHQNQQLVNQTHPNGVNFRFVHASPMSSYYASLNSGMNQYGQRETKNETQNGDISSLNNSSYPYPPNMGRVQSG

SEQ ID NO: 25 - DNA, 1728 bp, Glycine max
atggcatcttcttctggtagtttagacacctctgcaagtgcaaactccttcaccaacttcaccttctccacacaccctttcatgaccacttctttctctgacctccttgcttctcccttggacaacaacaagccaccacagggtggtttgtctgagagaactggctctggtgttcccaaattcaagtccacaccaccaccttctctgcctctctctccccctcccatttctccttcttcttactttgctattcctcctggtttgagccctgctgagcttcttgactcgccggttctccttaactcttccaacattctgccatctccaacaactggagcatttgttgctcagagcttcaattggaagagcagttcaggggggaatcagcaaattgtcaaggaagaagacaaaagcttctcaaatttctctttccaaacccgatcaggacctcctgcttcatccacagcaacataccagtcttcaaatgtcacagttcaaacacaacagccatggagttttcaggaggccacgaaacaggataatttttcctcaggaaagggtatgatgaaaactgaaaactcttcttccatgcagagtttttcccctgagattgctagtgtccaaactaaccatagcaatgggtttcaatccgattatggcaattaccccccacaatctcagactttaagtagaaggtcagatgatgggtacaattggaggaaatatggccaaaaacaagtgaagggaagtgaaaatccaagaagttattacaaatgcacataccccaattgccctacaaagaagaaggttgagaggtctttagatggacaaattactgagatagtttataagggtactcataaccatcctaagcctcaaaatactaggagaaactcatcaaactcctcttctcttgcaatccctcattcaaattccatcagaactgaaatcccagatcaatcctatgccacacatggaagtggacaaatggattcagctgccaccccagaaaactcatcaatatcaattggagatgatgattttgagcagagttcccaaaagtgtaaatcaggaggggatgaatatgatgaagatgaacctgatgccaaaagatggaaaattgaaggtgaaaatgagggtatgtcagcccctggaagtagaacagtgagagaacctagagttgtagttcagacaaccagtgacattgatatccttgatgatggctataggtggagaaaatacgggcagaaagtagtgaagggcaatccaaatccaaggagttactacaagtgcacacacccaggatgtccagtgaggaagcacgtggaaagagcctcacatgacctaagggctgtgatcacaacttatgagggaaagcacaaccatgatgttcctgcagcccgtggcagtggcagccattctgtgaacagaccaatgccaaacaatgcttcaaaccacaccaacactgcagccacttccgtaaggctcttgccagtgatccaccaaagtgacaattcccttcagaaccaaagatcacaagcaccaccagaagggcaatcacccttcaccctagagatgctacaaagtccaggaagttttggattctcagggtttgggaatccaatgcaatcttacgtgaaccagcagcaactatctgacaatgttttctcctccaggaccaaggaggagcctagagatgacatgttccttgagtctctactatgctga

SEQ ID NO: 26 - protein, 574 aa, Glycine max
MASSSGSLDTSASANSFTNFTFSTHPFMTTSFSDLLASPLDNNKPPQGGLSERTGSGVPKFKSTPPPSLPLSPPPISPSSYFAIPPGLSPAELLDSPVLLNSSNILPSPTTGAFVAQSFNWKSSSGGNQQIVKEEDKSFSNFSFQTRSGPPASSTATYQSSNVTVQTQQPWSFQEATKQDNFSSGKGMMKTENSSSMQSFSPEIASVQTNHSNGFQSDYGNYPPQSQTLSRRSDDGYNWRKYGQKQVKGSENPRSYYKCTYPNCPTKKKVERSLDGQITEIVYKGTHNHPKPQNTRRNSSNSSSLAIPHSNSIRTEIPDQSYATHGSGQMDSAATPENSSISIGDDDFEQSSQKCKSGGDEYDEDEPDAKRWKIEGENEGMSAPGSRTVREPRVVVQTTSDIDILDDGYRWRKYGQKVVKGNPNPRSYYKCTHPGCPVRKHVERASHDLRAVITTYEGKHNHDVPAARGSGSHSVNRPMPNNASNHTNTAATSVRLLPVIHQSDNSLQNQRSQAPPEGQSPFTLEMLQSPGSFGSGFGNPMQSYVNQQQLSDNVFSSRTKEEPRDDMFLESLLC

SEQ ID NO: 27 - DNA, 1578 bp, Solanum chacoense
atggcttcttcaggtggaaatatgaacagtttcatgaattcatttaactcattttcttcttctcaattcatgacttcttcgtttagcgatcttctttctgataataatgataacaacaataagaattggggatttagcgatgacagaattaagtcatttccaatgattaactcatcttcatcaccggcttcgccttcttcttatcttgcttttccgcattctttaagtccatcaatgcttttggactcccctgttttgttcaacaattcaaatactctttcatcaccaacatcagggagttttggtaatttgaattctaaagaggggaattcaaggagttctgaattttctttccaaagtaggcctgctacttcatcctcaatctttcaatcttctgctccaagaaactcattggaagacttaatgacaaggcaacaacagacaactgaattctccacagcaaaaactggagtgaaatcagaagtggctccaattcaaagcttttctcaagagaacatgccgaataatcctgctccggtgcattactgtcaaccatcgcaatatgttagagaacagaaggcggaagatggatataattggaggaaatatggacaaaagcaagtgaaaggaagtgagaatccgcgaagttattacaagtgtacatttcccaattgtcctacaaagaagaaggttgaaaggaacttggatggacacattactgagatagtttataaggggagccataatcatccaaaacctcaatccactagaagatcatcttcacaatcgattcaaaaccttgcttactccaacttggatgtaacaaatcagccaaacgcgtttcatgaaaatggtcaaagagactcctttgctgtcacagacaattcttcagcttcttttggagatgaggatgttgatcaaggctctcctatcagtaagtcaggagaaaatgatgaaaatgaacccgaggcaaagagatggaagggtgacaatgaaaatgaggttatatcatctgcaagtagaacagtacgtgaacctagaatcgtagttcaaaccacaagcgacattgatattcttgatgatggttatagatggagaaaatatggacaaaaagttgtcaaaggaaatccaaacccaaggagttactacaagtgcacatttactggatgtccggttaggaagcatgtggaacgagcatctcatgatctaagagcagttatcacaacttatgaaggaaaacacaaccatgatgttcctgcagcacgtggtagtggtagctatgcaatgaatagacctccaactggaagcaacaacaacatgccagtagttcctaggcctacagtgttggctaatcattctaatcagggaatgaattttaatgacacattttttaacacaacgcagatccaaccaccaatcaccctgcagatgctacagagctctggaagttcaagttattcaggatttggtaactcatcgggatcttacatgaatcaaatgcagcacacgaacaattccaagccgataagcaaagaagaacctaaagatgatttattcttcagctctttccttaactga

SEQ ID NO: 28 - protein, 525 aa, Solanum chacoense
MASSGGNMNSFMNSFNSFSSSQFMTSSFSDLLSDNNDNNNKNWGFSDDRIKSFPMINSSSSPASPSSYLAFPHSLSPSMLLDSPVLFNNSNTLSSPTSGSFGNLNSKEGNSRSSEFSFQSRPATSSSIFQSSAPRNSLEDLMTRQQQTTEFSTAKTGVKSEVAPIQSFSQENMPNNPAPVHYCQPSQYVREQKAEDGYNWRKYGQKQVKGSENPRSYYKCTFPNCPTKKKVERNLDGHITEIVYKGSHNHPKPQSTRRSSSQSIQNLAYSNLDVTNQPNAFHENGQRDSFAVTDNSSASFGDEDVDQGSPISKSGENDENEPEAKRWKGDNENEVISSASRTVREPRIVVQTTSDIDILDDGYRWRKYGQKVVKGNPNPRSYYKCTFTGCPVRKHVERASHDLRAVITTYEGKHNHDVPAARGSGSYAMNRPPTGSNNNMPVVPRPTVLANHSNQGMNFNDTFFNTTQIQPPITLQMLQSSGSSSYSGFGNSSGSYMNQMQHTNNSKPISKEEPKDDLFFSSFLN

SEQ ID NO: 29 - DNA, 1650 bp, Ipomoea batatas
atggctgcttcttcagggacaatagacgcccccacagcttcttcatctttctctttctccaccgcctcttcattcatgtcctctttcactgacctcctcgcttccgacgcctattccggcggctctgtgagcagagggcttggtgatcggatagcggagagaactgggtcgggtgtgcccaagtttaagtctttgccgccgccgtctctgccgctttcctcgccggccgtctcgccgtcgtcttacttcgcttttccgcctgggttgagccccagtgagctcctggattcccctgttcttctatcttcctcaaacattttgccgtctccaactactgggacttttcctgctcagaccttcaactggaagaatgattctaacgcatcccaggaagatgttaagcaagaagagaaaggatacccagatttctcttttcagaccaactctgcttcaatgacattgaattatgaagattcaaagaggaaagatgagctcaattctctgcagagccttccccctgtgactacttcaactcagatgagctctcagaacaatggtgggagctactctgagtataataatcaatgctgcccgccttcccagacgttgagggagcagaggcgatctgatgacgggtacaattggaggaaatacgggcagaaacaggtgaaggggagcgaaaatccgaggagttattacaagtgtacgcacccgaattgccccacgaagaagaaggtcgagagggctttggatgggcagattactgagattgtctacaaaggagctcacaatcacccgaagcctcagtccactaggagatcgtcgtcctctacagcttcttcggcttcaaccttggctgcccagtcttataatgcgcctgccagtgatgtcccggatcagtcgtactggtctaatggtaacgggcagatggattctgttgccacgccagagaattcttcgatctcggtgggggatgatgaattcgagcagagctctcagaagagggagtccgggggagacgagtttgatgaagacgaaccggatgcaaagagatggaaagtggaaaacgaaagcgagggagtttctgcacaggggagtaggacagtaagagaaccgagagttgtagttcaaacgacgagtgatattgatattcttgacgatggttatagatggagaaaatatggccagaaagttgtgaagggaaatcccaatccaaggagctattacaaatgcacgagccaaggctgtccggtgaggaaacacgtggaaagagcttcacacgatatccgctcggtgataacaacctacgaaggaaaacacaaccacgacgtccctgctgcccgagggagtggcagccacggcctcaaccggggcgccaatcctaacaacaatgcggccatggccatggcgattaggccttcgacgatgtctctccaatctaactatcccatcccaatcccgagcacgaggccaatgcagcagggagaaggccaagcgccttacgagatgttgcagggatcgggcggttttgggtactcgggatttgggaacccgatgaacgcctacgcgaaccaaatccaggacaacgcgttctcgagggccaaggaggagcccagagatgacttgtttctggacacattgctagcttga

SEQ ID NO: 30 - protein, 549 aa, Ipomoea batatas
MAASSGTIDAPTASSSFSFSTASSFMSSFTDLLASDAYSGGSVSRGLGDRIAERTGSGVPKFKSLPPPSLPLSSPAVSPSSYFAFPPGLSPSELLDSPVLLSSSNILPSPTTGTFPAQTFNWKNDSNASQEDVKQEEKGYPDFSFQTNSASMTLNYEDSKRKDELNSLQSLPPVTTSTQMSSQNNGGSYSEYNNQCCPPSQTLREQRRSDDGYNWRKYGQKQVKGSENPRSYYKCTHPNCPTKKKVERALDGQITEIVYKGAHNHPKPQSTRRSSSSTASSASTLAAQSYNAPASDVPDQSYWSNGNGQMDSVATPENSSISVGDDEFEQSSQKRESGGDEFDEDEPDAKRWKVENESEGVSAQGSRTVREPRVVVQTTSDIDILDDGYRWRKYGQKVVKGNPNPRSYYKCTSQGCPVRKHVERASHDIRSVITTYEGKHNHDVPAARGSGSHGLNRGANPNNNAAMAMAIRPSTMSLQSNYPIPIPSTRPMQQGEGQAPYEMLQGSGGFGYSGFGNPMNAYANQIQDNAFSRAKEEPRDDLFLDTLLA

SEQ ID NO: 31 - DNA, 1692 bp, Nicotiana attenuata
atggcttcttcaggtggaaatatgaatacttttatgaattcttttactagcaactattctttttcatcttttagtgaccttctttctgataataataataataataataataataataatatgaggagtaataacagttctatgaaccaagaaaagaattcattgaattggggtttcagtgatcaaagaatgaatcaacaaaacaaagatgaagttccaaagtttaagtcatttccaccttgttctttgccaatgatttcatcttcatcaccagcttctccttcttcttatcttgcttttcctccttctttaagtccatctgtgcttttggactcaccagttttgtttaacaattccaatactcttccatctccaactacagggagttttggtaatttgaattcaaaggaggataattcaaagatttctgatttctctttccaaagtagggctgctacttcatcatcaatgtttcagtcttctgctccaagaaactcattggaagacttaatgacaagacaacaacatgccaaccagcaaaatgaattctccactgcaaaaactacaggggtgaaatcagaagtggctcaaattcaaagcttctcccaagaaaagatgcagagttatccagctccagtacattacactcaaccatctcaatatgttagggaacagaaagcagaagatggatacaactggaggaaatatgggcagaagcaagtgaaaggaagcgagaatccgcgaagctattacaagtgcacatttcctaactgtcctacaaagaagaaggttgaaaggaatttggatggacacattactgagatagtctataaggggaaccataatcatccaaagcctcagtccactagaagatcgtcttcacaatcgattcaaaaccttgcttactccaacttggatataacaaatcagccaaacgcgtttcttgataatgctcaaagggattcctttgctgggacagacaattcttcggcttcttttggtgacgaggatgttgatcaagggtctcctatcagcaagtcaggagaagatgatggaaatgaacccgaggcaaagagatggaagtgtgacaatgaaaacgaggtcatatcgtctgcaagtagaacagtaagagaaccaagaattgtagttcaaaccacaagcgacattgatattcttgacgacggttatagatggagaaaatatggacaaaaagttgtcaaaggcaatccaaatccaaggagctactacaaatgcacatttactggctgtccagttaggaagcatgtggaacgcgcttctcatgatctaagggcggtgatcacaacttatgaaggaaaacacaaccatgatgttcctgcagcacgtggtagtggaagctacgctatgaataaacctccatcaggcaacagcaacaacagcatgcctgtcgttccaaggccttcgatgttggctaacaactctaaccagggaatgaattttaacgacacatttttcaacacaagggtacaaacaacacagaaccaaccacctatcaccctgcagatgttacagagctctggaagttcaagttattcaggatttgatacctcctcggggtcttacatggaccaaatgcagcccatgaataataccaagccgataagcaaagaagaacctaaagatgatttattcttcagctctttccttaactga

SEQ ID NO: 32 - protein, 563 aa, Nicotiana attenuata
MASSGGNMNTFMNSFTSNYSFSSFSDLLSDNNNNNNNNNNMRSNNSSMNQEKNSLNWGFSDQRMNQQNKDEVPKFKSFPPCSLPMISSSSPASPSSYLAFPPSLSPSVLLDSPVLFNNSNTLPSPTTGSFGNLNSKEDNSKISDFSFQSRAATSSSMFQSSAPRNSLEDLMTRQQHANQQNEFSTAKTTGVKSEVAQIQSFSQEKMQSYPAPVHYTQPSQYVREQKAEDGYNWRKYGQKQVKGSENPRSYYKCTFPNCPTKKKVERNLDGHITEIVYKGNHNHPKPQSTRRSSSQSIQNLAYSNLDITNQPNAFLDNAQRDSFAGTDNSSASFGDEDVDQGSPISKSGEDDGNEPEAKRWKCDNENEVISSASRTVREPRIVVQTTSDIDILDDGYRWRKYGQKVVKGNPNPRSYYKCTFTGCPVRKHVERASHDLRAVITTYEGKHNHDVPAARGSGSYAMNKPPSGNSNNSMPVVPRPSMLANNSNQGMNFNDTFFNTRVQTTQNQPPITLQMLQSSGSSSYSGFDTSSGSYMDQMQPMNNTKPISKEEPKDDLFFSSFLN

SEQ ID NO: 33 - DNA, 1512 bp, Saccharum officinarum
atggcgtcctcgacggggagcttggagcacggggggttcacgttcacgccgccgcccttcatcacctcgttcacggagctgctctccggggcaggggacgtgctaggagccggcggcgccgatcaggagcggtcgccgaggggtctgttccaccgcggcgccaggggaggcggcggcgtgggcgtgcccaagttcaagtccgcgcagccgcccagcctgcccatctcgccgccgccgatgtcgccgtcctcctacttcgccatcccggccgggctcagccccgccgagctgctcgactcgcccgtcctgctccactattccgctaacaccttggcgtctcccaccaccggcgccatcccggcgcagaggttcgactggaagcaggccgccgatctgatcgcatctcagcaagacgacagccgttctggtgccatcggcggcttcaacgacttctccttccacacggccacctccaacgccatgcccgcgcagacgacgtccttcccttccttcaagcaggaacagcagcagcagcaagtcgaagccgcagcgaccaataagagcgccgtcgtcgcgtcgagcaacaagcaggcgagcagcggtggcgggaacagcaacaccaagctggaggacggctacaactggcgcaagtacgggcagaagcaggtgaaggggagcgagaacccgcgcagctactacaagtgcacgtaccacagctgctccatgaagaagaaggtggagcggtccctggccgacggccgcatcacgcagatcgtgtacaagggcgcgcacaaccaccccaagccgctgtccacgcgccgcaactcctccggcggcgtagccgcggcggaggagcagcaggccgccaacaacagcctctccgccgcggcggcgggctgcgggccggagcactccggcgccaccgccgagaactcgtccgtcaccttcggcgacgacgaggcggagaacgggtcgcaccggagcgatggcgacgagcccgacgccaagcgctggaagcaggaggatggtgagaacgagggcagctctggcggcgccggcggcaagccggtgcgcgagccccggctggtggtgcagacgctgagcgacatcgacatcctggacgacgggttccggtggcgcaagtacgggcagaaagtggtgaaggggaacccgaacccgcggagctactacaagtgcaccacggtggggtgccccgtgcggaagcacgtggagcgcgcgtcccacgacacgcgcgccgtgatcaccacgtacgagggcaagcacaaccacgacgtgcccgtgggccgcggtgcctcaagccgcgcggcggcggcggcggtggcgccgacgggctccgggtcagccttaatggccgccgccggcggccagctgggccatcagcagcagcagccctacaccctggagatgctgagcggcggagcatacggcggcggctacgcggccaaggacgagccgcgggacgacctgttcgtcgactcgctcctctgctag

SEQ ID NO: 34 - protein, 503 aa, Saccharum officinarum
MASSTGSLEHGGFTFTPPPFITSFTELLSGAGDVLGAGGADQERSPRGLFHRGARGGGGVGVPKFKSAQPPSLPISPPPMSPSSYFAIPAGLSPAELLDSPVLLHYSANTLASPTTGAIPAQRFDWKQAADLIASQQDDSRSGAIGGFNDFSFHTATSNAMPAQTTSFPSFKQEQQQQQVEAAATNKSAVVASSNKQASSGGGNSNTKLEDGYNWRKYGQKQVKGSENPRSYYKCTYHSCSMKKKVERSLADGRITQIVYKGAHNHPKPLSTRRNSSGGVAAAEEQQAANNSLSAAAAGCGPEHSGATAENSSVTFGDDEAENGSHRSDGDEPDAKRWKQEDGENEGSSGGAGGKPVREPRLVVQTLSDIDILDDGFRWRKYGQKVVKGNPNPRSYYKCTTVGCPVRKHVERASHDTRAVITTYEGKHNHDVPVGRGASSRAAAAAVAPTGSGSALMAAAGGQLGHQQQQPYTLEMLSGGAYGGGYAAKDEPRDDLFVDSLLC

SEQ ID NO: 35 - DNA, 1371 bp, Triticum aestivum
atgtcctcccccacagggagcttggatcacgcagggttcacgttcacgccgccgccgttcatcacgtccttgactgagcttctatcagggtctggcgccggcgacgtggaggggtcgccaagggggttcaaccgagggggtcgggccggggtgcccaagttcaagtccgcgcagccgcccagcctgcccatctcgtcgccagcgtcgcccttctcctgcttctccatccctgcaggtctcagccctgctgagctgctccactctcccgttctcctcaactactctcatatattggcgtctccgactacaggtgcgatccctgcgcggagatacgactggcaggcgagcgccgatctgaacacctttcagcaggatgaggtcggccgtggagacagcggcctctttggcttctcctttcacgcagtgaagcccaacgccacggtcaacgctcaaacaaattacttacctttattcaaggaacatcagcagcagcaacaacaacaacaagtggtagaagtgagcaacaagagcagcagcggcgacaacaacaagcaggttgaggacggatacaattggaggaagtacgggcagaagcaagttaaggggagcgagaacccgcggagctactacaagtgcacctacaacaattgctccatgaagaagaaagtggagcgctctctcgccgacggccgcatcacgcagatcgtctacaagggcgcacatgaccacccgaagcccctctccacgcgccgcaacttctccggctgcgcggcggtcgttgcggaggatcataccaacggctcggagcactctggcccgacgcccgagaattcatccgtcactttcggagacgatgaggccgacaagcccgagaccaagcgccggaaggagcatggtgacaacgagggcagttcaggcggcaccggcggctgcgggaagcccgtgcgcgagcccaggctcgtggtgcagacgctgagcgatatagacatactcgacgacggcttccggtggaggaagtacgggcagaaggttgtcaagggcaatcccaaccccaggagctactacaagtgcacaacggtgggctgcccggtgcgcaagcatgtggagcgggcctcgcacgacaaccgcgcggtgattaccacctacgagggtaggcacagccacgacgtgccggtcggcaggggggccggtgccagccgcgcgctgccgacgtcgtcttcctccgacagctcggtcgtcgtctgtcctgccgccgccgggcaggccccgtacaccctcgagatgctcgccaaccctgccgccggacaccgaggctacgcggccaaggacgaaccccgggacgacatgttcgtcgagtcgctcctctgctag

SEQ ID NO: 36 - protein, 456 aa, Triticum aestivum
MSSPTGSLDHAGFTFTPPPFITSLTELLSGSGAGDVEGSPRGFNRGGRAGVPKFKSAQPPSLPISSPASPFSCFSIPAGLSPAELLHSPVLLNYSHILASPTTGAIPARRYDWQASADLNTFQQDEVGRGDSGLFGFSFHAVKPNATVNAQTNYLPLFKEHQQQQQQQQVVEVSNKSSSGDNNKQVEDGYNWRKYGQKQVKGSENPRSYYKCTYNNCSMKKKVERSLADGRITQIVYKGAHDHPKPLSTRRNFSGCAAVVAEDHTNGSEHSGPTPENSSVTFGDDEADKPETKRRKEHGDNEGSSGGTGGCGKPVREPRLVVQTLSDIDILDDGFRWRKYGQKVVKGNPNPRSYYKCTTVGCPVRKHVERASHDNRAVITTYEGRHSHDVPVGRGAGASRALPTSSSSDSSVVVCPAAAGQAPYTLEMLANPAAGHRGYAAKDEPRDDMFVESLLC

SEQ ID NO: 37 - DNA, 1722 bp, Hordeum vulgare
atgtcccccgcgcggctgccgatctcgcgcgagtcctgcctcaccatccccgccggcttcagcccctcagcgctcctcgactcccccgtgctcctcaccaacttcaaggttgaaccttcaccaacaactggtagtctgggcatggctgcgattctgcacaagagcgctcatccagacatgctgccttcgccacgggataaatctgttcgtaatgcccatgaagataggggttctagggattttgaattcaagcctcatctgaattcgtcttctcaatcactggctcctgctatgagtgatctaaaaaaacatgagcattctatgcaaaatcagagtatgaatcccagctcatcatctagcaatatggtgaatgaaaacagacctccctgttcacgtgagtcgagtcttacagtgaatgtaagtgctcagaaccaacctgttggaatggttggtttgactgacagcatgcctgctgaagttggtacatctgagccgcagcagatgaatagctctgacaatgccatgcaagagccgcagtctgaaaatgttgctgacaagtcggcagatgatggctacaactggcggaaatacgggcagaagcatgtcaagggaagtgaaaaccctagaagttactacaagtgcacacatcctaattgtgaagtaaaaaagctattggagcgtgcagttgatggtctgatcacggaagttgtctataagggacgccacaatcatcctaagccccagcccaataggaggttagctggtggtgcagttccttcaaaccagggtgaagaacgatatgacggcgcttcagctgctgatgataaatcttccaatgctcttagcaaccttgctaatccggtacattcgcctggtatggttgagcctgttccagcttcagttagtgatgatgacatcgatgctggaggtggaagaccctaccctggggatgatgctactgaggaggaggatttagagtcgaaacgcaggaaaatggagtctgctggtattgatgctgctctgatgggtaaacctaaccgtgagccccgtgtcgtcgttcaaactgtaagtgaggttgacatcttggatgatggctatcgttggcggaaatatggacagaaagttgtcaaaggaaaccccaatccacggagttactacaaatgcacaagcacaggatgccctgtgaggaagcatgttgagagagcatcacacgatcctaaatcagtgataacaacgtatgaaggaaaacataaccatgaagtccctgctgcgaggaatgcaacccatgagatgtccgcgcctcccatgaagaacgtcgtgcatcagattaacagcaatatgcccagcagcattggtggcatgatgagggcatgtgaagccaggaactacaccaaccaatattctcaggcggctgaaaccgacactgtcagtcttgatcttggtgttggaatcagcccaaaccacagcgacgcgacaaaccaaatgcagtcttcaggtcctgaccagatgcagtatcaaatgcaaaccatgggttcgatgtacggcaacatgagacatccatcatcaatggcagcgccagcggtacaaggaaactctgctgcccgcatgtatggttcgagagaagagaaaggtaacgaagggtttactttcagagccacaccgatggaccattcagctaacctatgctatagcagtgctgggaacttggtcatgggtccatga

SEQ ID NO: 38 - protein, 573 aa, Hordeum vulgare
MSPARLPISRESCLTIPAGFSPSALLDSPVLLTNFKVEPSPTTGSLGMAAILHKSAHPDMLPSPRDKSVRNAHEDRGSRDFEFKPHLNSSSQSLAPAMSDLKKHEHSMQNQSMNPSSSSSNMVNENRPPCSRESSLTVNVSAQNQPVGMVGLTDSMPAEVGTSEPQQMNSSDNAMQEPQSENVADKSADDGYNWRKYGQKHVKGSENPRSYYKCTHPNCEVKKLLERAVDGLITEVVYKGRHNHPKPQPNRRLAGGAVPSNQGEERYDGASAADDKSSNALSNLANPVHSPGMVEPVPASVSDDDIDAGGGRPYPGDDATEEEDLESKRRKMESAGIDAALMGKPNREPRVVVQTVSEVDILDDGYRWRKYGQKVVKGNPNPRSYYKCTSTGCPVRKHVERASHDPKSVITTYEGKHNHEVPAARNATHEMSAPPMKNVVHQINSNMPSSIGGMMRACEARNYTNQYSQAAETDTVSLDLGVGISPNHSDATNQMQSSGPDQMQYQMQTMGSMYGNMRHPSSMAAPAVQGNSAARMYGSREEKGNEGFTFRATPMDHSANLCYSSAGNLVMGP

SEQ ID NO: 39 - protein, 76 aa, Oryza sativa
  MISC_FEATURE: conserved motif
REPRLVVQTLSDIDILDDGFRWRKYGQKVVKGNPNPRSYYKCTTVGCPVRKHVERASHDTRAVITTYEGKHNHDVP

SEQ ID NO: 40 - DNA, 50 bp, Artificial sequence
  primer prm05769
ggggacaagtttgtacaaaaaagcaggcttaaacaatggcgtcctcgacg

SEQ ID NO: 41 - DNA, 47 bp, Artificial sequence
  primer prm05770
ggggaccactttgtacaagaaagctgggtggctcgactagcagagga

SEQ ID NO: 42 - DNA, 2193 bp, Oryza sativa
aatccgaaaagtttctgcaccgttttcaccccctaactaacaatatagggaacgtgtgctaaatataaaatgagaccttatatatgtagcgctgataactagaactatgcaagaaaaactcatccacctactttagtggcaatcgggctaaataaaaaagagtcgctacactagtttcgttttccttagtaattaagtgggaaaatgaaatcattattgcttagaatatacgttcacatctctgtcatgaagttaaattattcgaggtagccataattgtcatcaaactcttcttgaataaaaaaatctttctagctgaactcaatgggtaaagagagagattttttttaaaaaaatagaatgaagatattctgaacgtattggcaaagatttaaacatataattatataattttatagtttgtgcattcgtcatatcgcacatcattaaggacatgtcttactccatcccaatttttatttagtaattaaagacaattgacttatttttattatttatcttttttcgattagatgcaaggtacttacgcacacactttgtgctcatgtgcatgtgtgagtgcacctcctcaatacacgttcaactagcaacacatctctaatatcactcgcctatttaatacatttaggtagcaatatctgaattcaagcactccaccatcaccagaccacttttaataatatctaaaatacaaaaaataattttacagaatagcatgaaaagtatgaaacgaactatttaggtttttcacatacaaaaaaaaaaagaattttgctcgtgcgcgagcgccaatctcccatattgggcacacaggcaacaacagagtggctgcccacagaacaacccacaaaaaacgatgatctaacggaggacagcaagtccgcaacaaccttttaacagcaggctttgcggccaggagagaggaggagaggcaaagaaaaccaagcatcctcctcctcccatctataaattcctccccccttttcccctctctatataggaggcatccaagccaagaagagggagagcaccaaggacacgcgactagcagaagccgagcgaccgccttcttcgatccatatcttccggtcgagttcttggtcgatctcttccctcctccacctcctcctcacagggtatgtgcccttcggttgttcttggatttattgttctaggttgtgtagtacgggcgttgatgttaggaaaggggatctgtatctgtgatgattcctgttcttggatttgggatagaggggttcttgatgttgcatgttatcggttcggtttgattagtagtatggttttcaatcgtctggagagctctatggaaatgaaatggtttagggtacggaatcttgcgattttgtgagtaccttttgtttgaggtaaaatcagagcaccggtgattttgcttggtgtaataaaagtacggttgtttggtcctcgattctggtagtgatgcttctcgatttgacgaagctatcctttgtttattccctattgaacaaaaataatccaactttgaagacggtcccgttgatgagattgaatgattgattcttaagcctgtccaaaatttcgcagctggcttgtttagatacagtagtccccatcacgaaattcatggaaacagttataatcctcaggaacaggggattccctgttcttccgatttgctttagtcccagaattttttttcccaaatatcttaaaaagtcactttctggttcagttcaatgaattgattgctacaaataatgcttttatagcgttatcctagctgtagttcagttaataggtaatacccctatagtttagtcaggagaagaacttatccgatttctgatctccatttttaattatatgaaatgaactgtagcataagcagtattcatttggattattttttttattagctctcaccccttcattattctgagctgaaagtctggcatgaactgtcctcaattttgttttcaaattcacatcgattatctatgcattatcctcttgtatctacctgtagaagtttctttttggttattccttgactgcttgattacagaaagaaatttatgaagctgtaatcgggatagttatactgcttgttcttatgattcatttcctttgtgcagttcttggtgtagcttgccactttcaccagcaaagttc

SEQ ID NO: 43 - DNA, 1236 bp, Oryza sativa
ggtcagccaatacattgatccgttgccaatcatgcaaagtattttggctgtggccgagtgccggaattgataattgtgttctgactaaattaaatgaccagaagtcgctatcttccaatgtatccgaaacctggattaaacaatcctgttctgttctctagcccctcctgcatggccggattgtttttttgacatgttttcttgactgaggcctgtttgttctaaactttttcttcaaacttttaactttttcatcacatcagaacttttctacacatataaacttttaacttttccgtcacatcgttccaatttcaatcaaactttcaattttggcgtgaactaaacacaccctgagtcttttattgctcctccgtacgggttggctggttgagaataggtattttcagagagaaaatctagatattgggaggaacttggcatgaatggccactatatttagagcaattctacggtccttgaggaggtaccatgaggtaccaaaattttagtgtaaattttagtatctcattataactaggtattatgaggtaccaaatttacaatagaaaaaatagtacttcatggtactttcttaagtaccgtaaaattgctcctatatttaaggggatgtttatatctatccatatccataatttgattttgataagaaaaaatgtgagcacaccaagcatgtccatgaccttgcactcttggctcactcgtcaactgtgaagaacctcaaaaatgctcaatatagctacaggtgcctgaaaaaataactttaaagttttgaacatcgatttcactaaacaacaattattatctccctctgaaagatgatagtttagaactctagaatcattgtcggcggagaaagtaaattattttccccaaatttccagctatgaaaaaaccctcaccaaacaccatcaaacaagagttcaccaaaccgcccatgcggccatgctgtcacgcaacgcaccgcattgcctgatggccgctcgatgcatgcatgcttccccgtgcacatatccgacagacgcgccgtgtcagcgagctcctcgaccgacctgtgtagcccatgcaagcatccacccccgccacgtacaccccctcctcctccctacgtgtcaccgctctctccacctatatatgcccacctggcccctctcctcccatctccacttcacccgatcgcttcttcttcttcttcgttgcattcatcttgctagc

SEQ ID NO: 44 - DNA, 1470 bp, Zea mays
atggcgtcctcgacggggagcttggagcacggcgggttcacgttcacgccgccgcccttcatcacctccttcacggagctgctctctggggcagcagcggacatggtaggagccgcgggcgccgatcatcaggagcggtcgccgagggggctgttccaccgcggcgccaccaggggcggcggcgtaggcgtgcccaagttcaagtcggcgcagccgcccagcctgcccatctcgccgccgccgatgtcgccctcctcctacttctccatcccgcccgggctcagccccgccgagctgctggactcgcccgtcctgctccactcttcctcgaacttcttcgcgtcgcccaccaccggcgccatcccggcgcagaggttcgactggaagcatgccgccgacctgatcgcgtctcagtctcagcaagacgacagccgggctgcggtcggcagcgccttcaacgacttctctttccacgcgcccaccatgcccgcgcagaccacgtccttcccttccttcaaggagcagcagcaacagcaagtcgaagcggcgaccaagagcgccgtcccgtcgagcaacaaggccagcggcggcggcggcggcaccaagcttgaggacgggtacaactggcgcaagtacgggcagaagcaggttaaggggagcgagaacccgcgcagctactacaagtgcacgtaccacagctgctccatgaagaagaaggtggagcggtccctggccgacgggcgcgtcacgcagatcgtgtacaagggcgcgcacaaccaccccaagccgctgtccacgcgccgcaactcctccggcggcgtggcggcggcggaggagcaggccgccaacaacagcagcctctctggctgcggcggcccggagcactccggcggcgccaccgccgagaactcgtccgtcacgttcggcgacgacgaggcggagaacgggtcgcagcggagcggcggtgacgagcccgacgccaagcgctggaaggcggaggacggtgagaacgaaggcagctcaggcgccggcggcggcaagccggtgcgcgagccgcggctggtggtacagacgctgagcgacatcgacatcctggacgacgggttccggtggcgcaagtacgggcagaaggtggtgaaggggaacccgaacccgcggagctactacaagtgcaccacggcggggtgccccgtgcggaagcacgtggagcgcgcgtgccacgacgcgcgcgccgtgatcaccacgtacgagggcaagcacaaccacgacgtgcccgtgggccgcggcgccgccagccgcgcggcggccgccgcgccactgctgggctccggcggcggccagatggatcatcgtcatcagcagccctacaccctggagatgctgagcggcggaggaggagggtacggcggcggctacgcggccaaggacgagccgcgggacgacctgttcgtcgactcgctcctctgctag

SEQ ID NO: 45 - protein, 489 aa, Zea mays
MASSTGSLEHGGFTFTPPPFITSFTELLSGAAADMVGAAGADHQERSPRGLFHRGATRGGGVGVPKFKSAQPPSLPISPPPMSPSSYFSIPPGLSPAELLDSPVLLHSSSNFFASPTTGAIPAQRFDWKHAADLIASQSQQDDSRAAVGSAFNDFSFHAPTMPAQTTSFPSFKEQQQQQVEAATKSAVPSSNKASGGGGGTKLEDGYNWRKYGQKQVKGSENPRSYYKCTYHSCSMKKKVERSLADGRVTQIVYKGAHNHPKPLSTRRNSSGGVAAAEEQAANNSSLSGCGGPEHSGGATAENSSVTFGDDEAENGSQRSGGDEPDAKRWKAEDGENEGSSGAGGGKPVREPRLVVQTLSDIDILDDGFRWRKYGQKVVKGNPNPRSYYKCTTAGCPVRKHVERACHDARAVITTYEGKHNHDVPVGRGAASRAAAAAPLLGSGGGQMDHRHQQPYTLEMLSGGGGGYGGGYAAKDEPRDDLFVDSLLC

SEQ ID NO: 46 - DNA, 1608 bp, Lycopersicon esculentum
atggctgcttcaagtttctcttttcccacttcatcttcttcattcatgacgacttctttcaccgaccttcttgcttctgatgattatccaaccaaaggacttgctgatagaattgcagagaggactggttctggagttcctaaattcaaatctcttccacctccttcacttccattatcgcctcctcctttttcgccttcctcttactttgctattcctcctggtttaagtccaactgaacttttagactcccctgttcttttgtcttcttcaaaccttcttccatctccgacgactgggagttttccatctcgtgcttttaattggaagagcagtagtcatcaggatgtgaaacaggaagacaaaaactactcagatttttctttccagcctcaagtagggacagctgcatcatcaatctctcaatctcaaactaaccatgtccctctggggcagcaagcatggaattgtcaagagcccacaaaacagaatgatcaaaatgctaatggaagatccgaattcaacactgtacagaattttatgcagaataataatgatcagaacaatagtggaaaccaatacaatcagagtataagggagcagaaaaggtcagatgacggatacaattggaggaaatacgggcagaaacaagtaaaaggtagtgaaaatccgagaagctactacaagtgtacatacccaaattgtcccaccaagaagaaggttgagagatctttagatggtcaaattactgaaattgtgtacaagggtaatcacaaccatccaaagcctcagtctaccagaagatcgtcatcatccacagcttcatctgcattccaatcttacaatacacaaactaatgaaattccagatcatcaatcctatggttcaaatggacaaatggattccgttgcaacacctgagaattcttcgatttcatttggggatgatgatcatgaacacacttctcaaaagagtagtaggtcaagaggagatgatcttgatgaagaggaaccagactcaaaaagatggaaaagagaaaacgaaagtgaaggtgtatctgcactaggagggagtaggacagttagagaacctagagttgtagttcaaactacgagtgacatcgatatcctagatgatggttatagatggaggaagtatggtcaaaaagtagtgaaaggaaatcctaatcccaggagctactacaaatgcacaagtacgggatgtccagtaagaaaacatgtggaaagggcatcacaagacataaggtcagtgataacaacctatgaagggaagcacaaccatgatgttccagcagcaaggggcagtggcaaccactcaattaaccgacctatggcaccgaccataaggcctactgtgacatctcatcaatccaactatcaagttccattacaaagtataaggccacaacagtctgaaatgggagcaccctttacactagagatgttgcagaagcctaataattatggtttctcaggatatgcaaattcaggggattcatatgaaaaccaagttcaggacaataatgtgttttcgagaactaaggacgagcctcgagatgacttgtttatggagtcattgctttgctga

SEQ ID NO: 47 - protein, 535 aa, Lycopersicon esculentum
MAASSFSFPTSSSSFMTTSFTDLLASDDYPTKGLADRIAERTGSGVPKFKSLPPPSLPLSPPPFSPSSYFAIPPGLSPTELLDSPVLLSSSNLLPSPTTGSFPSRAFNWKSSSHQDVKQEDKNYSDFSFQPQVGTAASSISQSQTNHVPLGQQAWNCQEPTKQNDQNANGRSEFNTVQNFMQNNNDQNNSGNQYNQSIREQKRSDDGYNWRKYGQKQVKGSENPRSYYKCTYPNCPTKKKVERSLDGQITEIVYKGNHNHPKPQSTRRSSSSTASSAFQSYNTQTNEIPDHQSYGSNGQMDSVATPENSSISFGDDDHEHTSQKSSRSRGDDLDEEEPDSKRWKRENESEGVSALGGSRTVREPRVVVQTTSDIDILDDGYRWRKYGQKVVKGNPNPRSYYKCTSTGCPVRKHVERASQDIRSVITTYEGKHNHDVPAARGSGNHSINRPMAPTIRPTVTSHQSNYQVPLQSIRPQQSEMGAPFTLEMLQKPNNYGFSGYANSGDSYENQVQDNNVFSRTKDEPRDDLFMESLLC

SEQ ID NO: 48 - DNA, 1623 bp, Lycopersicon esculentum
atggcttcttcaggtggaaatatgaacacttttatgaattcatttaactcattttcttcttctcaattcatgacttcttcgtttagcgatcttctttcggacaataataataataataatgataataagaattggggatttagcgacgatagaattaagtcatttccaatgatgaactcatcaacatcacctgcttcgccttcctcttatcttgcttttccgcattcattaagtccatctatgcttttggactcacccgttttgttcaacaattctaatactcttcaatcgccaactacagggagttttggtaatttgaactctaaagaggggaattcaaggaattctgaattttcttttcaaagtaggcctgctacttcttcctcaatcttccaatcttctgctccaagaaactcattggaagacttaatgacaaggcaacaacagaccactgaattctccacagcaaaaactggagtgaaatcagaagtggctccaattcaaagcttttcacatgagaacatgtcgaataatcctgctccggtgcattactgtcaaccatcgcaatatgttagagaacagaaggcggaagatggatataattggaggaaatatggacaaaagcaagtgaaaggaagtgaaaatccgcgaagttattacaagtgtacatttccaaattgtcctacaaagaagaagggtgaaaggaacttggatggacacattactgagatagtttataaagggagtcataatcatcccaaacctcaatccactagaagatcatcttcacaatcgattcaaaaccttgcttactccaacttggatgtaacaaaccagccaaacgcgtttcttgaaaatggtcaaagagactcctttgctgttacagacaattcttcagcttcttttggagatgacgatgttgatcaaggctctcctatcagtaaatcaggagaaaatgatgaaaatgaacccgaggcaaagagatggaagggtgacaatgaaaacgaggttatatcatctgcaagtagaacagtacgtgaacctagaatcgtagttcaaaccacaagtgacattgatatacttgatgatggttatagatggagaaaatacggtcaaaaagttgtcaaaggaaatccaaacccaaggagttactacaagtgcacatttactggatgtccggttaggaagcatgtggaacgagcatctcatgatctaagagccgttatcacaacttatgaaggaaaacacaaccatgatgttcctgcagcacgtggtagtggtagctacgcaatgaataaacctccatctggaagcaacaacaacaacaataacatgccagtagttccaaggcctatagtgttggctaaccattctaatcagggaatgaactttaacgacacatttttcaacacaacacagatccaaccaccaatcaccctccagatgctacagagctctggaacttcaagttattcaggatttggtaactcatcgggatcctacatgaatcaaatgcagcacacaaacaattccaagccgataagcaaagaagaacccaaagatgatttattcttcagctctttccttaactggaactcctccataccaaaatacaaaagatag

SEQ ID NO: 49 - protein, 540 aa, Lycopersicon esculentum
MASSGGNMNTFMNSFNSFSSSQFMTSSFSDLLSDNNNNNNDNKNWGFSDDRIKSFPMMNSSTSPASPSSYLAFPHSLSPSMLLDSPVLFNNSNTLQSPTTGSFGNLNSKEGNSRNSEFSFQSRPATSSSIFQSSAPRNSLEDLMTRQQQTTEFSTAKTGVKSEVAPIQSFSHENMSNNPAPVHYCQPSQYVREQKAEDGYNWRKYGQKQVKGSENPRSYYKCTFPNCPTKKKGERNLDGHITEIVYKGSHNHPKPQSTRRSSSQSIQNLAYSNLDVTNQPNAFLENGQRDSFAVTDNSSASFGDDDVDQGSPISKSGENDENEPEAKRWKGDNENEVISSASRTVREPRIVVQTTSDIDILDDGYRWRKYGQKVVKGNPNPRSYYKCTFTGCPVRKHVERASHDLRAVITTYEGKHNHDVPAARGSGSYAMNKPPSGSNNNNNNMPVVPRPIVLANHSNQGMNFNDTFFNTTQIQPPITLQMLQSSGTSSYSGFGNSSGSYMNQMQHTNNSKPISKEEPKDDLFFSSFLNWNSSIPKYKR

SEQ ID NO: 50 - DNA, 1641 bp, Zea mays
atggcgtcctcgacggggagcttggagcacggcgggttcacgttcacgccgccgcccttcatcacctccttcacggagctgctctctggggcagcagcggacatggtaggagccgcgggcgccgatcatcaggagcggtcgccgagggggctgttccaccgcggcgccaccaggggcggcggcgtaggcgtgcccaagttcaagtccgcgcagccgcccagcctgcccatctcgccgccgccgatgtcgccctcctcctacttctccatcccgcccgggctcagccccgccgagctgctggactcgcccgtcctgctccactcttcctcgaacttcttcgcgtcgcccaccaccggcgccatcccggcgcagaggttcgactggaagcaggccgccgacctgatcgcgtctcagtctcagcaagacgacagccgggctgcggtcggcagcgccttcaacgacttctccttccacgcgcccaccatgcccgcgcagaccacgtccttcccttccttcaaggagcagcagcaacagcaagtcgaagcggcgaccaagagcgccgtcccgtcgagcaacaaggccagcggcggaagcggcggcggcaccaagcttgaggacgggtacaactggcgcaagtacgggcagaagcaggtgaaggggagcgagaacccgcgcagctactacaagtgcacgtaccacagctgctccatgaagaagaaggtggagcggtccctggccgacgggcgcgtcacgcagatcgtgtacaagggcgcgcacaaccaccccaagccgctgtccacgcgccgcaactcctccggcggcgtggcggcggcggaggagcaggccgccaacaacagcagcctctctggctgcggcggcccggagcactccggcggcgccaccgccgagaactcgtccgtcacgttcggcgacgacgaggcggagaacgggtcgcagcggagcggcggtgacgagcccgacgccaagcgctggaaggcggaggacggtgagaacgaaggcagctcaggcgccggcggcggcaagccggtgcgcgagccgcggctggtggtacagacgctgagcgacatcgacatcctggacgacgggttccggtggcgcaagtacgggcagaaggtggtgaaggggaacccgaacccgcggagctactacaagtgcaccacggcggggtgccccgtgcggaagcacgtggagcgcgcgtgccacgacgcgcgcgccgtgatcaccacgtacgagggcaagcacaaccacgacgtgcccgtgggccgcggcgccgccagccgcgctgctgctgctgcggcggcgggttccggggccttgatggccaccggcggcggccagctcgggtatcagcagcaccagcagcagcagccatacaccctggagatgctgagcagcggatcgtacggcggcggcggctacgtgccccggcgccgccagccgagctgctgctgcggcggcggcggcttcgcgttctcctccggcttcgacaacccgatgggctcgtacatgagccagcaccagcagcagcagaggcagaacgacgccatgcacgcgtcgagggccaaggaggagccccgggaggacatgtttttcccgacgtcgttgctgtacactgactga

SEQ ID NO: 51 - protein, 546 aa, Zea mays
MASSTGSLEHGGFTFTPPPFITSFTELLSGAAADMVGAAGADHQERSPRGLFHRGATRGGGVGVPKFKSAQPPSLPISPPPMSPSSYFSIPPGLSPAELLDSPVLLHSSSNFFASPTTGAIPAQRFDWKQAADLIASQSQQDDSRAAVGSAFNDFSFHAPTMPAQTTSFPSFKEQQQQQVEAATKSAVPSSNKASGGSGGGTKLEDGYNWRKYGQKQVKGSENPRSYYKCTYHSCSMKKKVERSLADGRVTQIVYKGAHNHPKPLSTRRNSSGGVAAAEEQAANNSSLSGCGGPEHSGGATAENSSVTFGDDEAENGSQRSGGDEPDAKRWKAEDGENEGSSGAGGGKPVREPRLVVQTLSDIDILDDGFRWRKYGQKVVKGNPNPRSYYKCTTAGCPVRKHVERACHDARAVITTYEGKHNHDVPVGRGAASRAAAAAAAGSGALMATGGGQLGYQQHQQQQPYTLEMLSSGSYGGGGYVPRRRQPSCCCGGGGFAFSSGFDNPMGSYMSQHQQQQRQNDAMHASRAKEEPREDMFFPTSLLYTD

SEQ ID NO: 52 - protein, 560 aa, Helan WRKY 2X
MSFSSSSGITLETPPSSTPSFSFSMSSFSDQPPPPRTTGLAARIAERVGSGIPKFKSIPPPSLPISPPAVSPSSYFAIPAGLSPAELLDSPVLLSSSNILPSPTTGSFPFQAFNWKNLNGNFHNEEHSIKKEQKSLADFSFRPQLHHPTEQQIWNNQKQQIDQDEKSLTQSGHSPPMQSFSPEIATIQTDSNSQAQSFQSGYDTNSSSNFNNQTLQKKSEDGYNWRKYGQKQVKGSENPRSYYKCTYPNCSMKKKLETNIEGQITEIVYKGNHNHPKPQSTRRSSSSSASNTLQMSQASSNHDVHDYPDQSYVSHGSGQVDSVTTPENSSISVGDDEFDRSRSGGDGVTVDEDEPEAKRWKVSENEGISMIGGTKTVREPRIVVQTTSDIDILDDGYRWRKYGQKVVKGNPNPRSYYKCTSLGCSVRKHVERASQDLRSVITTYEGKHNHDVPMARGSGHRLQASTLSNNAPSMTIKPMALSHYQVDNSMVDPTRGPRYPPSSENQAPFTLEMLQSSDNFKYSRFENALKSNYNEHNSERTFSTTKEEPRDDMFFESLLF

(19) United States
(12) Patent Application Publication
(10) Pub. No.: US 2009/0106861 A1
(43) Pub. Date: Apr. 23, 2009

(54) Plants Having Increased Yield and a Method for Making the Same

(75) Inventor: Valerie Frankard, Waterloo (BE)

Correspondence Address: CONNOLLY BOVE LODGE & HUTZ, LLP, P O BOX 2207, WILMINGTON, DE, 19899, US

(73) Assignee: CROPDESIGN N.V., Zwijnaarde (BE)

(21) Appl. No.: 11/988,276

(22) PCT Filed: Jul. 4, 2006
(86) PCT No.: PCT/EP2006/006495

§ 371 Date: Jan. 23, 2008

Related U.S. Application Data
(60) Provisional application No. 60/704,415, filed on Aug. 2, 2005.
(60) Provisional application No. 60/730,356, filed on Oct. 27, 2005.

(30) Foreign Application Priority Data
Jul. 5, 2005 (EP) 05106076.2
Oct. 24, 2005 (EP) 05109907.5

(51) Int. Cl. C12N 15/82; A01H 5/00; C12N 15/29; A01H 5/10; C12N 5/10; C12Q 1/68; A01H 1/04

(52) U.S. Cl. 800/290; 800/298; 536/23.6; 435/320.1; 435/419; 435/6; 800/320; 800/320.1; 800/320.2; 800/320.3

(57) ABSTRACT

The present invention concerns a method for increasing plant yield by modulating expression in a plant of a nucleic acid encoding a polypeptide having two WRKY domains or a homologue of such polypeptide. One such method comprises introducing into a plant a two-WRKY domain nucleic acid or variant thereof. The invention also relates to transgenic plants having introduced therein a two-WRKY domain nucleic acid or variant thereof, which plants have increased yield relative to control plants. The present invention also concerns constructs useful in the methods of the invention. The invention additionally relates to specific nucleic acid sequences encoding for the aforementioned proteins having the aforementioned plant growth improving activity, nucleic acid constructs, vectors and plants containing said nucleic acid sequences.

The present invention relates generally to the field of molecular biology and concerns a method for increasing plant yield relative to control plants. More specifically, the present invention concerns a method for increasing plant yield comprising modulating expression in a plant of a nucleic acid encoding a polypeptide having two WRKY domains or a homologue of such polypeptide. The present invention also concerns plants having modulated expression of a nucleic acid encoding a polypeptide having two WRKY domains or a homologue of such polypeptide, which plants have increased yield relative to control plants. The invention also provides constructs useful in the methods of the invention.

The invention additionally relates to specific nucleic acid sequences encoding for the aforementioned proteins having the aforementioned plant growth improving activity, nucleic acid constructs, vectors and plants containing said nucleic acid sequences.

The ever-increasing world population and the dwindling supply of arable land available for agriculture fuels research towards improving the efficiency of agriculture. Conventional means for crop and horticultural improvements utilise selective breeding techniques to identify plants having desirable characteristics. However, such selective breeding techniques have several drawbacks, namely that these techniques are typically labour intensive and result in plants that often contain heterogeneous genetic components that may not always result in the desirable trait being passed on from parent plants. Advances in molecular biology have allowed mankind to modify the germplasm of animals and plants. Genetic engineering of plants entails the isolation and manipulation of genetic material (typically in the form of DNA or RNA) and the subsequent introduction of that genetic material into a plant. Such technology has the capacity to deliver crops or plants having various improved economic, agronomic or horticultural traits. A trait of particular economic interest is yield. Yield is normally defined as the measurable produce of economic value, necessarily related to a specified crop, area and/or period of time. This may be defined in terms of quantity and/or quality. Yield is directly dependent on several factors, for example, the number and size of the organs, plant architecture (for example, the number of branches), seed production and more. Root development, nutrient uptake and stress tolerance may also be important factors in determining yield. Optimizing one of the abovementioned factors may therefore contribute to increasing crop yield.

Plant biomass is yield for forage crops like alfalfa, silage corn and hay. Many proxies for yield have been used in grain crops. Chief amongst these are estimates of plant size. Plant size can be measured in many ways depending on species and developmental stage, but include total plant dry weight, above-ground dry weight, above-ground fresh weight, leaf area, stem volume, plant height, rosette diameter, leaf length, root length, root mass, tiller number and leaf number. Many species maintain a conservative ratio between the size of different parts of the plant at a given developmental stage. These allometric relationships are used to extrapolate from one of these measures of size to another (e.g. Tittonell et al 2005 Agric Ecosys & Environ 105: 213). Plant size at an early developmental stage will typically correlate with plant size later in development. A larger plant with a greater leaf area can typically absorb more light and carbon dioxide than a smaller plant and therefore will likely gain a greater weight during the same period (Fasoula & Tollenaar 2005 Maydica 50:39). This is in addition to the potential continuation of the micro-environmental or genetic advantage that the plant had to achieve the larger size initially. There is a strong genetic component to plant size and growth rate (e.g. ter Steege et al 2005 Plant Physiology 139:1078), and so for a range of diverse genotypes plant size under one environmental condition is likely to correlate with size under another (Hittalmani et al 2003 Theoretical Applied Genetics 107:679). In this way a standard environment is used as a proxy for the diverse and dynamic environments encountered at different locations and times by crops in the field.

Harvest index, the ratio of seed yield to above-ground dry weight, is relatively stable under many environmental conditions and so a robust correlation between plant size and grain yield can often be obtained (e.g. Rebetzke et al 2002 Crop Science 42:739). These processes are intrinsically linked because the majority of grain biomass is dependent on current or stored photosynthetic productivity by the leaves and stem of the plant (Gardener et al 1985 Physiology of Crop Plants. Iowa State University Press, pp 68-73) Therefore, selecting for plant size, even at early stages of development, has been used as an indicator for future potential yield (e.g. Tittonell et al 2005 Agric Ecosys & Environ 105: 213). When testing for the impact of genetic differences on stress tolerance, the ability to standardize soil properties, temperature, water and nutrient availability and light intensity is an intrinsic advantage of greenhouse or plant growth chamber environments compared to the field. However, artificial limitations on yield due to poor pollination due to the absence of wind or insects, or insufficient space for mature root or canopy growth, can restrict the use of these controlled environments for testing yield differences. Therefore, measurements of plant size in early development, under standardized conditions in a growth chamber or greenhouse, are standard practices to provide indication of potential genetic yield advantages.

The ability to increase plant yield would have many applications in areas such as agriculture, including in the production of ornamental plants, arboriculture, horticulture and forestry. Increasing yield may also find use in the production of algae for use in bioreactors (for the biotechnological production of substances such as pharmaceuticals, antibodies or vaccines, or for the bioconversion of organic waste) and other such areas.

Transcription factor polypeptides are usually defined as proteins that show sequence-specific DNA binding affinity and that are capable of activating and/or repressing transcription. WRKY proteins are a large family of plant-specific transcription factors, functioning either alone or as part of multimeric protein-DNA complexes. Most of these proteins are involved in defence against attack from a wide range of pathogens (Eulgem et al., EMBO J., 18, 1999: 4689-4699, Deslandes et al., Proc. Natl. Acad. Sci, USA, 99, 2002: 2404-2409, Li et al., Plant Cell 16, 2004: 319-331). Furthermore, WRKY proteins are involved in responses to abiotic stresses such as wounding (Yoda et al., Mol. Genet. Genomics, 267, 2002: 154-161), drought, heat and cold (Fowler et al., Plant Cell, 14, 2002: 1675-1690, Mare et al., Plant Mol. Biol., 55, 2004: 399-416). Some members of this family have also been shown to play important regulatory roles in trichome formation (Johnson et al., Plant Cell, 14, 2002: 1359-1375), senescence (Hinderhofer et al., Planta, 213, 2001: 469-473, Guo et al., Plant Cell Environ., 27, 2004: 521-549), dormancy and metabolic pathways.

WRKY proteins are a multi gene family. In Arabidopsis thaliana more than 74 members of the family are known (Uelker et al., Curr. Op. in Plant Biol., 7, 2004: 491-498). They contain at least one highly conserved WRKY domain, which typically consists of about 60 conserved amino acids. The WRKY domain comprises at its amino-terminal end a hallmark heptapeptide WRKYGQK (where Q in rare instances may be replaced by E or K) and at its carboxy-terminal end a zinc-finger motif distinct from other known zinc-finger motifs. To regulate gene expression (by activation and/or repression), the WRKY domain binds to cis-acting elements in the promoter of target genes, with a preference for the W box, but also to others such as the SURE or the SP8 elements (for review, see Eulgem et al. (2000) Trends Plant Sci 5(5): 199-206). The DNA binding can be block with metal chelators such as EDTA or o-phenatrolin and restored by adding zinc ions. WRKY transcription factors are belonging to the so-called “immediate early response” genes, that means they are involved in the rapid responses of plants to wounding, to pathogens or to inducers of disease resistance.

WRKY proteins have been classified into three major groups based on the number of WRKY domains and on the features of their associated zinc-finger motif.
Group I comprise proteins with two WRKY domains and a Cys₂His₂ (or C₂—H₂) zinc-finger motif (more precisely C—X₄₋₅—C—X₂₂₋₂₃—H—X₁—H) or Cys₂HisCys (or a C₂—HC) zinc-finger motif (more precisely C—X₇—C—X₂₃—H—X₁—C), where C is Cys, H is His, and X is any amino acid);
Group II (the largest group) comprise proteins with one WRKY domain and the same Cys₂His₂ zinc-finger motif as in group 1;
Group III comprise proteins with one WRKY domain but a Cys₂HisCys (or a C₂—HC) zinc-finger motif (more specifically C—X₄₋₅—C—X₂₂₋₂₃—H—X₁—C or C—X₇C—X₂₃—H—X₁—C, where is Cys, H is His, and X is any amino acid) instead of Cys₂His₂.

The rice genome is thought to encode over 100 proteins with at least one full WRKY domain, and at least 12 of these are reported to contain two WRKY domains (Zhang & Wang (2005) BMC Evolutionary Biology 5:1). In these 12, the carboxy-terminal WRKY domain is the site of the major DNA-binding activity, whereas the amino-terminal WRKY domain facilitates DNA-binding or engages in protein-protein interactions. The zinc-finger motif in each WRKY domain may be involved in binding to either DNA or proteins.

Like other transcription factors, WRKY proteins have an abundance of potential transcriptional activation or repression domains. A common feature of many domains affecting transcription is the predominance of certain amino acids, including alanine (Ala), glutamine (Glu), proline (Pro), serine (Ser), threonine (Thr) and charged amino acids. Another common feature likely to be encountered in WRKY proteins is a basic nuclear localisation signal (NLS), which usually consists of a short stretch of basic amino acid residues.

It has now been found that modulating expression in a plant of a nucleic acid encoding a polypeptide having two WRKY domains or a homologue of such polypeptide gives plants having increased yield relative to control plants.

According to one embodiment of the present invention, there is provided a method for increasing plant yield relative to control plants, comprising modulating expression in a plant of a nucleic acid encoding a polypeptide having two WRKY domains or a homologue of such polypeptide.

Advantageously, performance of the methods according to the present invention results in plants having increased yield, particularly seed yield, relative to control plants.

Preferably the polypeptide used in the inventive method has two WRKY domains or the homologue comprises from amino-terminus to carboxy-terminus: (i) a Pro-Ser rich domain; and (ii) two WRKY domains including a zinc-finger C₂—H₂ motif.

The choice of advantageous control plants is a routine part of an experimental setup and may include corresponding wild type plants or corresponding plants without the gene of interest. The control plant may also be a nullizygote of the plant to be compared. Nullizygotes are individuals missing the transgene by segregation. Preferably, the control plant is of the same species, more preferably of the same variety as the plant to be compared. A “control plant” as used herein refers not only to whole plants, but also to plant parts, including seeds and seed parts.

A “reference”, “reference plant”, “control”, “control plant”, “wild type” or “wild type plant” is in particular a cell, a tissue, an organ, a plant, or a part thereof, which was not produced according to the method of the invention. Accordingly, the terms “wild type”, “control” or “reference” are exchangeable and can be a cell or a part of the plant such as an organelle or tissue, or a plant, which was not modified or treated according to the herein described method according to the invention. Accordingly, the cell or a part of the plant such as an organelle or a plant used as wild type, control or reference corresponds to the cell, plant or part thereof as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property. That means in other words that the wild type denotes (1) a plant, which carries the unaltered or not modulated form of a gene or allele or (2) the starting material/plant from which the plants produced by the process or method of the invention are derived.

Preferably, any comparison between the wild type plants and the plants produced by the method of the invention is carried out under analogous conditions. The term “analogous conditions” means that all conditions such as, for example, culture or growing conditions, assay conditions (such as buffer composition, temperature, substrates, pathogen strain, concentrations and the like) are kept identical between the experiments to be compared.

The “reference”, “control”, or “wild type” is preferably a subject, e.g. an organelle, a cell, a tissue, a plant, which was not modulated, modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-” “control-” or “wild type-”-organelle, -cell, -tissue or plant, relates to an organelle, cell, tissue or plant, which is nearly genetically identical to the organelle, cell, tissue or plant, of the present invention or a part thereof preferably 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is preferably a subject, e.g. an organelle, a cell, a tissue, a plant, which is genetically identical to the plant, tissue, cell, organelle used according to the method of the invention except that nucleic acid molecules or the gene product encoded by them are changed, modulated or modified according to the inventive method.

In case a control, reference or wild type differing from the subject of the present invention only by not being subject of the method of the invention can not be provided, a control, reference or wild type can be a plant in which the cause for the modulation of the activity conferring the increase of the metabolites is as described under examples.

The term “yield” in general means a measurable produce of economic value, necessarily related to a specified crop, to an area, and to a period of time. Individual plant parts directly contribute to yield based on their number, size and/or weight. Whereas the actual yield is the yield per acre for a crop and year, which is determined by dividing total production (includes both harvested and appraised production) by planted acres.

The terms “increase”, “improving” or “improve” are interchangeable and shall mean in the sense of the application at least a 5%, 6%, 7%, 8%, 9% or 10%, preferably at least 15% or 20%, more preferably 25%, 30%, 35% or 40% more yield and/or growth in comparison to the wild type plant as defined herein.

The increase referred to the activity of the polypeptide amounts in a cell, a tissue, a organelle, an organ or an organism or a part thereof preferably to at least 5%, preferably to at least 10% or at to least 15%, especially preferably to at least 20%, 25%, 30% or more, very especially preferably are to at least 40%, 50% or 60%, most preferably are to at least 70% or more in comparison to the control, reference or wild type.

The term “increased yield” as defined herein is taken to mean an increase in any one or more of the following, each relative to control plants:
(i) increased biomass (weight) of one or more parts of a plant, particularly aboveground (harvestable) parts, increased root biomass or increased biomass of any other harvestable part;
(ii) increased total seed yield, which includes an increase in seed biomass (seed weight) and which may be an increase in the seed weight per plant or on an individual seed basis;
(iii) increased number of flowers (“florets”) per panicle
(iv) increased number of (filled) seeds;
(v) increased seed size, which may also influence the composition of seeds;
(vi) increased seed volume, which may also influence the composition of seeds (including oil, protein and carbohydrate total content and composition);
(vii) increased individual seed area;
(viii) increased individual seed length and/or width;
(ix) increased harvest index, which is expressed as a ratio of the yield of harvestable parts, such as seeds, over the total biomass; and
(x) increased thousand kernel weight (TKW), which is extrapolated from the number of filled seeds counted and their total weight. An increased TKW may result from an increased seed size and/or seed weight. An increased TKW may result from an increase in embryo size and/or endosperm size.

The term “expression” or “gene expression” means the transcription of a specific gene or specific genes. Preferably, this expression leads to the appearance of a phenotypic trait. The term “expression” or “gene expression” in particular means the transcription of a gene or genes into structural RNA (rRNA, tRNA) or mRNA with subsequent translation of the latter into a protein. The process includes transcription of DNA, processing of the resulting mRNA product and its translation into an active protein.

The term “modulation” means in relation to expression or gene expression, a process in which the expression level is changed by said gene expression in comparison to the control plant, preferably the expression level is increased. The original, unmodulated expression may be of any kind of expression of a structural RNA (rRNA, tRNA) or mRNA with subsequent translation. The term “modulating the activity” shall mean any change of the expression of the inventive nucleic acid sequences or encoded proteins, which leads to increased yield and/or increased growth of the plants.

Taking corn as an example, a yield increase may be manifested as one or more of the following: increase in the number of plants per hectare or acre, an increase in the number of ears per plant, an increase in the number of rows, number of kernels per row, kernel weight, TKW, ear length/diameter, among others. Taking rice as an example, a yield increase may be manifested by an increase in one or more of the following: number of plants per hectare or acre, number of panicles per plant, number of spikelets per panicle, number of flowers per panicle, increase in the seed filling rate, increase in TKW, among others. An increase in yield may also result in modified architecture, or may occur as a result of modified architecture.

According to a preferred feature, performance of the methods of the invention result in plants having increased seed yield relative to control plants.

In particular, such increased seed yield includes increased TKW, increased individual seed area, increased individual seed length, increased individual seed width, increased number of seeds and increased number of flowers per panicle, each relative to control plants.

Since the transgenic plants according to the present invention have increased yield, it is likely that these plants exhibit an increased growth rate (during at least part of their life cycle), relative to the growth rate of control plants at a corresponding stage in their life cycle. The increased growth rate may be specific to one or more parts of a plant (including seeds), or may be throughout substantially the whole plant. A plant having an increased growth rate may even exhibit early flowering. Delayed flowering is usually not a desirable agronomic trait. The increase in growth rate may take place at one or more stages in the life cycle of a plant or during substantially the whole plant life cycle. Increased growth rate during the early stages in the life cycle of a plant may reflect enhanced vigour. The increase in growth rate may alter the harvest cycle of a plant allowing plants to be sown later and/or harvested sooner than would otherwise be possible. If the growth rate is sufficiently increased, it may allow for the further sowing of seeds of the same plant species (for example sowing and harvesting of rice plants followed by sowing and harvesting of further rice plants all within one conventional growing period). Similarly, if the growth rate is sufficiently increased, it may allow for the further sowing of seeds of different plants species (for example the sowing and harvesting of rice plants followed by, for example, the sowing and optional harvesting of soy bean, potato or any other plant). Harvesting additional times from the same rootstock in the case of some crop plants may also be possible. Altering the harvest cycle of a plant may lead to an increase in annual biomass production per acre (due to an increase in the number of times (say in a year) that any particular plant may be grown and harvested). An increase in growth rate may also allow for the cultivation of transgenic plants in a wider geographical area than their wild-type counterparts, since the territorial limitations for growing a crop are often determined by adverse environmental conditions either at the time of planting (early season) or at the time of harvesting (late season). Such adverse conditions may be avoided if the harvest cycle is shortened. The growth rate may be determined by deriving various parameters from growth curves, such parameters may be: T-Mid (the time taken for plants to reach 50% of their maximal size) and T-90 (time taken for plants to reach 90% of their maximal size), amongst others.

Performance of the methods of the invention gives plants preferably having an increased growth rate. Therefore, according to the present invention, there is provided a method for increasing growth rate in plants, which method comprises modulating expression in a plant of a nucleic acid encoding a polypeptide having two WRKY domains or a homologue of such polypeptide.

An increase in yield and/or growth rate occurs whether the plant is under non-stress conditions or whether the plant is exposed to various stresses compared to control plants. Plants typically respond to exposure to stress by growing more slowly. In conditions of severe stress, the plant may even stop growing altogether. Mild stress on the other hand is defined herein as being any stress to which a plant is exposed which does not result in the plant ceasing to grow altogether without the capacity to resume growth. Mild stress in the sense of the invention leads to a reduction in the growth of the stressed plants of less than 40%, 35% or 30%, preferably less than 25%, 20% or 15%, more preferably less than 14%, 13%, 12%, 11% or 10% or less in comparison to the control plant under non-stress conditions. Due to advances in agricultural practices (irrigation, fertilization, pesticide treatments) severe stresses are not often encountered in cultivated crop plants. As a consequence, the compromised growth induced by mild stress is often an undesirable feature for agriculture. Mild stresses are the typical stresses to which a plant may be exposed, such as everyday biotic and/or abiotic (environmental) stresses. Typical abiotic or environmental stresses include temperature stresses caused by atypical hot or cold/freezing temperatures; salt stress; water stress (drought or excess water). Chemicals may also cause abiotic stresses. Biotic stresses are typically those stresses caused by pathogens, such as bacteria, viruses, fungi, nematodes and insects. Preferably an increase in yield and/or growth rate occurs according to the method of invention under non-stress or mild abiotic or biotic stress conditions, preferably abiotic stress conditions.

The abovementioned characteristics may advantageously be modified in any plant.

The term “plant” as used herein encompasses whole plants, ancestors and progeny of the plants and plant parts, including seeds, shoots, stems, leaves, roots (including tubers), fruits, stalk, seedlings, flowers, and cells, tissues and organs, wherein each of the aforementioned comprise genetic material not found in a wild type plant of the same species, variety or cultivar. The genetic material may be a transgene, an insertional mutagenesis event, an activation tagging sequence, a mutated sequence or a homologous recombination event. The term “plant” also encompasses suspension cultures, callus tissue, embryos, meristematic regions, gametophytes, sporophytes, pollen and microspores, again wherein each of the aforementioned comprise the genetic material not found in a wild type plant of the same species, variety or cultivar.

Plants that are particularly useful in the methods or processes of the invention include all plants which belong to the superfamily Viridiplantae, in particular monocotyledonous and dicotyledonous plants including fodder or forage legumes, ornamental plants, food crops, trees or shrubs selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chaenomeles spp., Cinnamomum cassia, Coffea arabica, Colophospemum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia spp., Eleusine coracana, Eragrestis spp., Erythrina spp., Eucalyptus spp., Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissoluta, Indigo incamata, Iris spp., Leptarrhena pyrolifolia, Lespediza spp., Lettuca spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativum, Podocarpus totara, Pogonarthria fleckii, Pogonarthria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys verticillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugarbeet, sugar cane, sunflower, tomato, squash, tea and algae, amongst others. According to a preferred embodiment of the present invention, the plant is a crop plant such as soybean, sunflower, canola, alfalfa, rapeseed, cotton, tomato, potato or tobacco. Further preferably, the plant is a monocotyledonous plant, such as sugar cane. More preferably the plant is a cereal, such as rice, maize, wheat, barley, millet, rye, sorghum or oats.

Other advantageous plants are selected from the group consisting of Asteraceae such as the genera Helianthus, Tagetes e.g. the species Helianthus annus [sunflower], Tagetes lucida, Tagetes erecta or Tagetes tenuifolia [Marigold], Brassicaceae such as the genera Brassica, Arabadopsis e.g. the species Brassica napus, Brassica rapa ssp. [canola, oilseed rape, turnip rape] or Arabidopsis thaliana. Fabaceae such as the genera Glycine e.g. the species Glycine max, Soja hispida or Soja max [soybean]. Linaceae such as the genera Linum e.g. the species Linum usitatissimum, [flax, linseed]; Poaceae such as the genera Hordeum, Secale, Avena, Sorghum, Oryza, Zea, Triticum e.g. the species Hordeum vulgare [barley]; Secale cereale [rye], Avena sativa, Avena fatua, Avena byzantina, Avena fatua var. sativa, Avena hybrida [oat], Sorghum bicolor [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat]; Solanaceae such as the genera Solanum, Lycopersicon e.g. the species Solanum tuberosum [potato], Lycopersicon esculentum, Lycopersicon lycopersicum, Lycopersicon pyriforme, Solanum integrifolium or Solanum lycopersicum [tomato].

The term “polypeptide having two WRKY domains or homologue of such polypeptide” as defined herein refers to a polypeptide comprising from amino-terminus to carboxy-terminus: (i) a Pro-Ser rich domain, and (ii) two WRKY domains including a zinc-finger C₂—H₂ motif.

Typically, the polypeptide having two WRKY domains or a homologue of such polypeptide may further comprise one or more of the following (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39.

The polypeptide having two WRKY domains or a homologue of such polypeptide may also comprise an LXSP motif within the Pro-Ser rich domain (where L is Leu, S is Ser, P is Pro and X is any amino acid). Furthermore, the Pro-Ser rich domain may be at least twice as rich in Pro and Ser compared to the average amino acid composition (in %) of Swiss-Prot Protein Sequence data bank proteins.

Furthermore, the polypeptide having two WRKY domains or a homologue of such polypeptide refers to any amino acid sequence which, when used in the construction of a phylogenetic tree of polypeptides comprising one or two WRKY domains, falls into the group which includes polypeptides having two WRKY domains and a Pro-Ser rich domain (see FIG. 2).

A polypeptide having two WRKY domains or homologue of such polypeptide is encoded by a two-WRKY domain nucleic acid/gene. Therefore the term “two-WRKY domain nucleic acid/gene” as defined herein is any nucleic acid/gene encoding a polypeptide having two WRKY domains or a homologue of such polypeptide as defined hereinabove.

Polypeptides having two WRKY domains or homologues of such polypeptides may readily be identified using routine techniques well known in the art, such as sequence alignment. Methods for the alignment of sequences for comparison are well known in the art, such methods include GAP, BESTFIT, BLAST, FASTA and TFASTA. GAP uses the algorithm of Needleman and Wunsch [(1970) J Mol Biol 48: 443-453] to find the alignment of two complete sequences that maximizes the number of matches and minimizes the number of gaps. The BLAST algorithm [Altschul et al. (1990) J Mol Biol 215: 403-10] calculates percent sequence identity and performs a statistical analysis of the similarity between the two sequences. The software for performing BLAST analysis is publicly available through the National Centre for Biotechnology Information. Homologues of a polypeptide having two WRKY domains may readily be identified using, for example, the ClustalW multiple sequence alignment algorithm (version 1.83) available at the Kyoto University Bioinformatics Center, with the default pairwise alignment parameters, and a scoring method in percentage. Some minimal manual editing may be required in some instances to optimise specific motif alignments; this is commonly carried out by persons skilled in the art. The sequence identity values, which are indicated above as a percentage were determined over the entire conserved domain using the programs mentioned above using the default parameters.

A person skilled in the art could readily determine whether any amino acid sequence in question falls within the aforementioned definition of a “polypeptide having two WRKY domains or homologue of such polypeptide” using known techniques and software for the making of a phylogenetic tree, such as a GCG, EBI or CLUSTAL package, using default parameters. Upon construction of such a phylogenetic tree, sequences clustering with the group of polypeptides having two WRKY domains and a Pro-Ser rich domain (see arrow in FIG. 2, after Eulgem et al., 2000, Trends Plant Sci 5(5): 199-206) will be considered to fall within the definition of a “polypeptide having two WRKY domains or homologue of such polypeptide”. Nucleic acids encoding such sequences will be useful in performing the methods of the invention.

The term “domain” refers to a set of amino acids conserved at specific positions along an alignment of sequences of evolutionarily related proteins. While amino acids at other positions can vary between homologues, amino acids that are highly conserved at specific positions indicate amino acids that are essential in the structure, the stability, or the activity of a protein. Identified by their high degree of conservation in aligned sequences of a family of protein homologues, they can be used as identifiers to determine if any polypeptide in question belongs to a previously identified polypeptide family (in this case, the family of polypeptides having two WRKY domains). The term “motif” refers a short conserved region in a protein sequence. Motifs are frequently highly conserved parts of domains, but may also include only part of the domain, or be outside of the conserved domain (if all of the amino acids of the motif fall outside of a defined domain).

Special databases exist for the identification of domains. The WRKY domains in a polypeptide may be identified using, for example, SMART (Schultz et al. (1998) Proc. Natl. Acad. Sci. USA 95, 5857-5864; Letunic et al., (2002) Nucleic Acids Res 30, 242-244; hosted by the EMBL at Heidelberg, Germany), InterPro (Mulder et al., (2003) Nucl. Acids. Res. 31, 315-318; hosted by the European Bioinformatics Institute (EBI) in the United Kingdom), Prosite (Bucher and Bairoch (1994), A generalized profile syntax for biomolecular sequences motifs and its function in automatic sequence interpretation. (In) ISMB-94; Proceedings 2nd International Conference on Intelligent Systems for Molecular Biology. Altman R., Brutlag D., Karp P., Lathrop R., Searls D., Eds., pp 53-61, AAAIPress, Menlo Park; Hulo et al., Nucl. Acids. Res. 32: D134-D137, (2004), The ExPASy proteomics server is provided as a service to the scientific community (hosted by the Swiss Institute of Bioinformatics (SIB) in Switzerland) or Pfam (Bateman et al., Nucleic Acids Research 30(1): 276-280 (2002), hosted by the Sanger Institute in the United Kingdom). In the InterPro database, the WRKY domain is designated by IPR003657, PF03106 in the Pfam database and PS50811 in the PROSITE database.

Furthermore, the presence of a Pro-Ser rich domain may also readily be identified. Primary amino acid composition (in %) to determine if a polypeptide domain is rich in specific amino acids may be calculated using software programs from the ExPASy server; in particular the ProtParam tool (Gasteiger E et al. (2003) ExPASy: the proteomics server for in-depth protein knowledge and analysis. Nucleic Acids Res 31:3784-3788). The composition of the protein of interest may then be compared to the average amino acid composition (in %) in the Swiss-Prot Protein Sequence data bank. Within this databank, the average Pro (P) content is of 4.85%, the average Ser (S) content is of 6.89%. As an example, the Pro-Ser rich domain of SEQ ID NO: 2 comprises 22.03% of Pro (more than 5 times enriched) and 20.34% of Ser (more than 3 times enriched). As defined herein, a Pro-Ser rich domain has a Pro and Ser content (in %) greater than that in the average amino acid composition (in %) in the Swiss-Prot Protein Sequence data bank. Further preferably, the Pro-Ser rich domain as defined herein has a Pro and Ser content (in %) that is at least double of the average amino acid composition (in %) in the Swiss-Prot Protein Sequence data bank. More preferably, the Pro-Ser rich domain as defined herein has a Pro and Ser content (in %) that is at least 2.1; 2.2; 2.3; 2.4 or 2.5, more preferably 2.6; 2.7; 2.8; 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more as much as the average amino acid composition (in %) of said kind of protein sequences, which are included in the Swiss-Prot Protein Sequence data bank.

Examples of polypeptides having two WRKY domains or homologues of such polypeptides include (encoded by polynucleotide sequence accession number in parenthesis; see also Table 1): Oryza sativa Orysa_WRKY53 (BK005056) SEQ ID NO: 2, Oryza sativa Orysa_WRKY24 (BK005027) SEQ ID NO: 4, Oryza sativa Orysa_WRKY70 (BK005073) SEQ ID NO: 6, Oryza sativa Orysa_WRKY78 (AK070537) SEQ ID NO: 8, Oryza sativa Orysa_WRKY30 (AY870610) SEQ ID NO: 10, Oryza sativa Orysa_WRKY35 (BK005038) SEQ ID NO: 12, Arabidopsis thaliana Arath_WRKY25 (NM_{—}128578) SEQ ID NO: 14, Arabidopsis thaliana Arath_WRKY26 (AK117545) SEQ ID NO: 16, Arabidopsis thaliana Arath_WRKY33 (NM_{—}129404) SEQ ID NO: 18, Arabidopsis thaliana Arath_WRKY2 (AF418308) SEQ ID NO: 20, Arabidopsis thaliana Arath_WRKY34 (AY052649) SEQ ID NO: 22, Arabidopsis thaliana Arath_WRKY20 (AF425837) SEQ ID NO: 24, Glycine max Glyma_WRKY 2X (contig of several ESTs among which BM143621.1, BU578260.1, CO036102.1) SEQ ID NO: 26, Solanum chacoense Solca_WRKY 2X (AY366389) SEQ ID NO: 28, Ipomoea batatas Ipoba_WRKY 2X (D30038) SEQ ID NO: 30, Nicotiana attenuata Nicta_WRKY 2X (AY456272) SEQ ID NO: 32, Saccharum officinarum Sacof_WRKY 2X SEQ ID NO: 34, Triticum aestivum Triae_WRKY 2X (contig of several EST's among which BM135197.1, BM138255.1, BT009257.1) SEQ ID NO: 36, Hordeum vulgare Horvu_WRKY 2X (AY323206) SEQ ID NO: 38, Zea mays Zeama_WRKY 2X (contig of CG310251.1, DR959456.1, DY235298.1) SEQ ID NO: 45, Lycopersicon esculentum Lyces_WRKY 2X (contig of CN385869.1, B1422509.1, CN38497745) SEQ ID NO: 47 and Lycopersicon esculentum Lyces WRKY 2X II (contig of B1422692.1, B1923269.1, B1422137.1) SEQ ID NO: 49 and the one mentioned in the sequence protocol under SEQ ID NO: 51 from Zea mays.

TABLE 1
Sequences falling under the definition of “polypeptide having two
WRKY domains or homologue of such polypeptide”.

| | NCBI | | Translated | |
|---|---|---|---|---|
| | accession | Nucleotide | polypeptide SEQ | |
| Name | number | SEQ ID NO | ID NO | Source |
| Orysa_WRKY53 | BK005056 | 1 | 2 | Oryza sativa |
| Orysa_WRKY24 | BK005027 | 3 | 4 | Oryza sativa |
| Orysa_WRKY70 | BK005073 | 5 | 6 | Oryza sativa |
| Orysa_WRKY78 | AK070537 | 7 | 8 | Oryza sativa |
| Orysa_WRKY30 | AY870610 | 9 | 10 | Oryza sativa |
| Orysa_WRKY35 | BK005038 | 11 | 12 | Oryza sativa |
| Arath_WRKY25 | NM_128578 | 13 | 14 | Arabidopsis thaliana |
| Arath_WRKY26 | AK117545 | 15 | 16 | Arabidopsis thaliana |
| Arath_WRKY33 | NM_129404 | 17 | 18 | Arabidopsis thaliana |
| Arath_WRKY2 | AF418308 | 19 | 20 | Arabidopsis thaliana |
| Arath_WRKY34 | AY052649 | 21 | 22 | Arabidopsis thaliana |
| Arath_WRKY20 | AF425837 | 23 | 24 | Arabidopsis thaliana |
| Glyma_WRKY | *contig of | 25 | 26 | Glycine max |
| 2X | several | | | |
| | EST's among | | | |
| | which | | | |
| | BM143621.1, | | | |
| | BU578260.1, | | | |
| | CO036102.1 | | | |
| Solca_WRKY 2X | AY366389 | 27 | 28 | Solanum chacoense |
| Ipoba_WRKY 2X | D30038 | 29 | 30 | Ipomoea batatas |
| Nicat_WRKY 2X | AY456272 | 31 | 32 | Nicotiana attenuata |
| Sacof_WRKY 2X | *contig of | 33 | 34 | Saccharum |
| | several | | | officinarum |
| | EST's among | | | |
| | which | | | |
| | CA096820.1, | | | |
| | CA119395.1, | | | |
| | CA139234.1 | | | |
| Triae_WRKY 2X | contig of | 35 | 36 | Triticum aestivum |
| | several | | | |
| | EST's among | | | |
| | which | | | |
| | CA731195.1, | | | |
| | CV764859.1, | | | |
| | BT009257.1 | | | |
| Horvu_WRKY | AY323206 | 37 | 38 | Hordeum vulgare |
| 2X | | | | |
| Zeama_WRKY | Contig of | 44 | 45 | Zea mays |
| 2X | CG310251.1 | | | |
| | DR959456.1 | | | |
| | DY235298.1 | | | |
| Lyces_WRKY 2X | Contig of | 46 | 47 | Lycopersicon |
| | CN385869.1 | | | esculentum |
| | BI422509.1 | | | |
| | CN384977 | | | |
| Lyces WRKY 2X | Contig of | 48 | 49 | Lycopersicon |
| II | BI422692.1 | | | esculentum |
| | BI923269.1 | | | |
| | BI422137.1 | | | |

It is to be understood that sequences falling under the definition of a “polypeptide having two WRKY domains or homologue of such polypeptide” are not to be limited to the amino acid sequences given in Table 1 and mentioned in the sequence protocol, but that any polypeptide comprising from amino-terminus to carboxy-terminus: (i) a Pro-Ser rich domain, and (ii) two WRKY domains including a zinc-finger C₂—H₂ motif, may be suitable for use in performing the methods of the invention.

Furthermore, the polypeptide having two WRKY domains or homologue of such polypeptide may also comprise one or more of the following (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39 (further exemplified in the Example 4). Even more preferably, the polypeptide having two WRKY domains or homologue of such polypeptide may further comprise an LXSP motif within the Pro-Ser rich domain (where L is Leu, S is Ser, P is Pro and where X is any amino acid). Most preferably, the polypeptide having two WRKY domains or a homologue of such polypeptide comprises a Pro-Ser rich domain at least twice as rich in Pro and Ser compared to the average amino acid composition (in %) of Swiss-Prot Protein Sequence data bank proteins. More preferably, the Pro-Ser rich domain as defined herein has a Pro and Ser content (in %) that is at least 2.1; 2.2; 2.3; 2.4 or 2.5, more preferably 2.6; 2.7; 2.8; 2.9 or 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more as much as the average amino acid composition (in %) of said kind of protein sequences, which are included in the Swiss-Prot Protein Sequence data bank.

Examples of two-WRKY domain nucleic acids include but are not limited to the nucleic acids given in Table 1 and mentioned in the sequence protocol. Two-WRKY domain nucleic acids/genes and variants thereof may be useful in practising the methods of the invention. Variant two-WRKY domain nucleic acid/genes include portions of a two-WRKY domain nucleic acid/gene and/or nucleic acids capable of hybridising with a two-WRKY domain nucleic acid/gene. SEQ ID NO: 1, SEQ ID NO: 50 or variants thereof are preferred for use in the methods of the present invention.

A further embodiment of the invention is an isolated nucleic acid molecule comprising a nucleic acid molecule selected from the group consisting of:
a) an isolated nucleic acid molecule as depicted in SEQ ID NO: 50;
b) an isolated nucleic acid molecule encoding the amino acid sequence as depicted in SEQ ID NO: 51;
c) an isolated nucleic acid molecule whose sequence can be deduced from a polypeptide sequence as depicted in SEQ ID NO: 51 as a result of the degeneracy of the genetic code;
d) an isolated nucleic acid molecule which encodes a polypeptide which has at least 80% identity with the amino acid sequence of the polypeptide encoded by the nucleic acid molecule of (a) to (c);
e) an isolated nucleic acid molecule encoding a homologue, derivative or active fragment of the amino acid molecule as depicted in SEQ ID NO: 51, which homologue, derivative or fragment is of plant origin and comprises advantageously
   (i) an acidic stretch between the two WRKY domains where at least 3 out of 6 amino acids are either Asp (D) or Glu (E);
   (ii) a putative NLS between the two WRKY domains where at least 3 out of 4 amino acids are either Lys (K) or Arg (R); and
   (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39;
f) an isolated nucleic acid molecule capable of hybridising with a nucleic acid of (a) to (c) above, or its complement, wherein the hybridising sequence or the complement thereof encodes the plant protein of (a) to (e);
   whereby the nucleic acid molecule has yield and/or growth increasing activities in plants.

For the purposes of the invention, “transgenic”, “transgene” or “recombinant” means with regard to, for example, a nucleic acid sequence, an expression cassette (=gene construct) or a vector comprising the nucleic acid sequence or an organism transformed with the nucleic acid sequences, expression cassettes or vectors according to the invention, all those constructions brought about by recombinant methods in which either
a) the nucleic acid sequences according to the invention, or
b) genetic control sequences which is operably linked with the nucleic acid sequence according to the invention, for example a promoter, or
c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide having two WRKY domains or a homologue of such polypeptide—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815.

A transgenic plant for the purposes of the invention is therefore understood as meaning, as above, that the nucleic acids used in the method of the invention are not at their natural locus in the genome of said plant, it being possible for the nucleic acids to be expressed homologously or heterologously. However, as mentioned, transgenic also means that, while the nucleic acids according to the invention or used in the inventive method are at their natural position in the genome of a plant, the sequence has been modified with regard to the natural sequence, and/or that the regulatory sequences of the natural sequences have been modified. Transgenic expression is preferably understood as meaning the expression of the nucleic acids according to the invention at an unnatural locus in the genome, i.e. homologous or, preferably, heterologous expression of the nucleic acids takes place. Preferred transgenic plants are mentioned herein.

Host plants for the nucleic acids, the expression cassette or the vector used in the method according to the invention or for the inventive nucleic acids, the expression cassette or construct or vector are, in principle, advantageously to all plants, which are capable of synthesizing the polypeptides used in the inventive method.

Unless otherwise specified, the terms “polynucleotides”, “nucleic acid” and “nucleic acid molecule” as used herein are interchangeably. Unless otherwise specified, the terms “peptide”, “polypeptide” and “protein” are interchangeably in the present context. The term “sequence” may relate to polynucleotides, nucleic acids, nucleic acid molecules, amino acids, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms refer only to the primary structure of the molecule.

Thus, the terms “gene(s)”, “polynucleotide”, “nucleic acid sequence”, “nucleotide sequence”, or “nucleic acid molecule(s)” as used herein include double- and single-stranded DNA and RNA. They also include known types of modifications, for example, methylation, “caps”, substitutions of one or more of the naturally occurring nucleotides with an analog. Preferably, the DNA or RNA sequence of the invention comprises a coding sequence encoding the herein defined polypeptide.

A “coding sequence” is a nucleotide sequence, which is transcribed into structural RNA or mRNA and/or translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

An “isolated” polynucleotide or nucleic acid molecule is separated from other polynucleotides or nucleic acid molecules, which are present in the natural source of the nucleic acid molecule. An isolated nucleic acid molecule may be a chromosomal fragment of several kb, or preferably, a molecule only comprising the coding region of the gene. Accordingly, an isolated nucleic acid molecule of the invention may comprise chromosomal regions, which are adjacent 5′ and 3′ or further adjacent chromosomal regions, but preferably comprises substantially few such sequences which naturally flank the nucleic acid molecule sequence in the genomic or chromosomal context in the organism from which the nucleic acid molecule originates (for example sequences which are adjacent to the regions encoding the 5′- and 3′-UTRs of the nucleic acid molecule). In various embodiments, the isolated nucleic acid molecule used in the process according to the invention may, for example comprise less than approximately 5 kb, 4 kb, 3 kb, 2 kb, 1 kb, 0.5 kb or 0.1 kb nucleotide sequences which naturally flank the nucleic acid molecule in the genomic DNA of the cell from which the nucleic acid molecule originates.

A nucleic acid molecule encompassing a complete sequence of the nucleic acid molecules used in the process, for example the polynucleotide of the invention, or a part thereof may additionally be isolated by polymerase chain reaction, oligonucleotide primers based on this sequence or on parts thereof being used. For example, a nucleic acid molecule comprising the complete sequence or part thereof can be isolated by polymerase chain reaction using oligonucleotide primers which have been generated on the basis of this very sequence. For example, mRNA can be isolated from cells (for example by means of the guanidinium thiocyanate extraction method of Chirgwin et al. (1979) Biochemistry 18:5294-5299) and cDNA can be generated by means of reverse transcriptase (for example Moloney MLV reverse transcriptase, available from GibcolBRL, Bethesda, Md., or AMV reverse transcriptase, obtainable from Seikagaku America, Inc., St. Petersburg, Fla.).

Nucleic acid molecules which are advantageously for the process according to the invention can be isolated based on their homology to the nucleic acid molecules disclosed herein using the sequences or part thereof as hybridization probe and following standard hybridization techniques under stringent hybridization conditions. In this context, it is possible to use, for example, isolated nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the method of the invention or encoding a protein used in the invention or of the nucleic acid molecule of the invention. Nucleic acid molecules with 30, 50, 100, 250 or more nucleotides may also be used.

The nucleic acid sequences used in the process of the invention, which are depicted in the sequence protocol in particular SEQ ID NO: 1 or 50 are advantageously introduced in a nucleic acid construct, preferably an expression cassette, which makes the expression of the nucleic acid molecules in a plant possible.

Accordingly, the invention also relates to a nucleic acid construct, preferably to an expression construct, comprising the nucleic acid molecule of the present invention functionally linked to one or more regulatory elements or signals.

As described herein, the nucleic acid construct can also comprise further genes, which are to be introduced into the organisms or cells. It is possible and advantageous to introduce into, and express in, the host organisms regulatory genes such as genes for inductors, repressors or enzymes, which, owing to their enzymatic activity, engage in the regulation of one or more genes of a metabolic pathway. These genes can be of heterologous or homologous origin. Moreover, further biosynthesis genes may advantageously be present, or else these genes may be located on one or more further nucleic acid constructs. Genes, which are advantageously employed are genes which influence the growth of the plants such as regulator sequences or factors. An enhancement of the regulator elements may advantageously take place at the transcriptional level by using strong transcription signals such as promoters and/or enhancers. In addition, however, an enhancement of translation is also possible, for example by increasing mRNA stability or by inserting a translation enhancer sequence.

In principle, the nucleic acid construct can comprise the herein described regulator sequences and further sequences relevant for the expression of the comprised genes. Thus, the nucleic acid construct of the invention can be used as expression cassette and thus can be used directly for introduction into the plant, or else they may be introduced into a vector. Accordingly in one embodiment the nucleic acid construct is an expression cassette comprising a microorganism promoter or a microorganism terminator or both. In one embodiment the expression cassette encompasses a viral promoter or a viral terminator or both. In another embodiment the expression cassette encompasses a plant promoter or a plant terminator or both.

To introduce a nucleic acid molecule into a nucleic acid construct, e.g. as part of an expression cassette, the gene segment is advantageously subjected to an amplification and ligation reaction in the manner known by a skilled person. It is preferred to follow a procedure similar to the protocol for the Pfu DNA polymerase or a Pfu/Taq DNA polymerase mixture. The primers are selected according to the sequence to be amplified. The primers should expediently be chosen in such a way that the amplificate comprise the codogenic sequence from the start to the stop codon. After the amplification, the amplificate is expediently analyzed. For example, the analysis may consider quality and quantity and be carried out following separation by gel electrophoresis. Thereafter, the amplificate can be purified following a standard protocol (for example Qiagen). An aliquot of the purified amplificate is then available for the subsequent cloning step. The skilled worker generally knows suitable cloning vectors.

They include, in particular, vectors which are capable of replication in easy to handle cloning systems like as bacterial yeast or insect cell based (e.g. baculovirus expression) systems, that is to say especially vectors which ensure efficient cloning in E. coli or Agrobacterium strains, and which make it possible to stably transform plants. Vectors, which must be mentioned, in particular are various binary and cointegrated vector systems, which are suitable for the T-DNA-mediated transformation. Such vector systems are generally characterized in that they contain at least the vir genes, which are required for the Agrobacterium-mediated transformation, and the T-DNA border sequences.

In general, vector systems preferably also comprise further cis-regulatory regions such as promoters and terminators and/or selection markers by means of which suitably transformed organisms can be identified. While vir genes and T-DNA sequences are located on the same vector in the case of cointegrated vector systems, binary systems are based on at least two vectors, one of which bears vir genes, but no T-DNA, while a second one bears T-DNA, but no vir gene. Owing to this fact, the last-mentioned vectors are relatively small, easy to manipulate and capable of replication in E. coli and in Agrobacterium strains. These binary vectors include vectors from the series pBIB-HYG, pPZP, pBecks, pGreen. Those which are preferably used in accordance with the invention are Bin19, pBI101, pBinAR, pGPTV and pCAMBIA. An overview of binary vectors and their use is given by Hellens et al, Trends in Plant Science (2000) 5, 446-451. The vectors are preferably modified in such a manner, that they already contain the nucleic acids of the invention, preferentially the nucleic acid sequences encoding the polypeptides as depicted in SEQ ID NO: 1 and SEQ ID NO: 50.

In a recombinant expression vector, “operable linkage” means that the nucleic acid molecule of interest is linked to the regulatory signals in such a way that expression of the nucleic acid molecule is possible: they are linked to one another in such a way that the two sequences fulfil the predicted function assigned to the sequence (for example in an in-vitro transcription/translation system, or in a host cell if the vector is introduced into the host cell).

The term portion as defined herein refers to a piece of DNA encoding a polypeptide that performs the same or similar biological functions to the intact polypeptide. For example, a two-WRKY domain portion may encode a polypeptide comprising a recognizable structural motif and/or functional domain such as a DNA-binding site or domain that binds to a DNA promoter region, an activation or repression domain, a domain for protein-protein interactions, a localization domain and may also have the ability to initiate or inhibit transcription. A portion may be prepared, for example, by making one or more deletions to a two-WRKY domain nucleic acid. The portions may be used in isolated form or they may be fused to other coding (or non coding) sequences in order to, for example, produce a protein that combines several activities. When fused to other coding sequences, the resulting polypeptide produced upon translation may be bigger than that predicted for the two-WRKY domain portion. Examples of portions may include the nucleotides encoding a polypeptide comprising from amino-terminus to carboxy-terminus: (i) a Pro-Ser rich domain, and (ii) two WRKY domains including a zinc-finger C₂—H₂ motif. Portions may optionally comprise any one or more of the following: (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39. The portion may further comprise an LXSP motif within the Pro-Ser rich domain (where L is Leu, S is Ser, P is Pro and X is any amino acid). The portion is typically at least 300, 400, 500, 600 or 700 nucleotides in length, preferably at least 750, 900, 850, 900 or 950 nucleotides in length, more preferably at least 1000, 1100, 1200 or 1300 nucleotides in length and most preferably at least 1350, 1400, 1450, 1500, 1550 or 1600 nucleotides or more in length. Preferably, the portion is a portion of any one of the nucleic acids given in Table 1 and/or mentioned in the sequence protocol. Most preferably the portion is a portion of a nucleic acid as represented by SEQ ID NO: 1 or SEQ ID NO: 50.

The terms “fragment”, “fragment of a sequence” or “part of a sequence” “portion” or “portion thereof” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence referred to or hybidizing with the nucleic acid molecule of the invention or used in the process of the invention under stringend conditions, while the maximum size is not critical. In some applications, the maximum size usually is not substantially greater than that required to provide the desired activity and/or function(s) of the original sequence. A comparable function means at least 40%, 45% or 50%, preferably at least 60%, 70%, 80% or 90% or more of the original sequence.

Another variant of a two-WRKY domain nucleic acid/gene is a nucleic acid capable of hybridising under reduced stringency conditions, preferably under stringent conditions, most preferably under highly stringent conditions, with a two-WRKY domain nucleic acid/gene as hereinbefore defined. The hybridising sequence may include the nucleotides encoding a polypeptide comprising from amino-terminus to carboxy-terminus: (i) a Pro-Ser rich domain, and (ii) two WRKY domains including a zinc-finger C₂—H₂ motif. The hybridising sequence may optionally comprise any one or more of the following: (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39. The hybridising sequence may further comprise an LXSP motif within the Pro-Ser rich domain (where L is Leu, S is Ser, P is Pro and X is any amino acid). The hybridising sequence is typically at least 100, 125, 150, 175, 200 or 225 nucleotides in length, preferably at least 250, 275, 300, 325, 350, 375, 400, 425, 450 or 475 nucleotides in length, further preferably least 500, 525, 550, 575, 600, 625, 650, 675, 700 or 725 nucleotides in length, more preferably at least 750, 800, 900, 1000, 1100, 1200 or 1300 nucleotides in length and most preferably at least 1400 nucleotides or more in length. Preferably, the hybridising sequence is one that is capable of hybridising to any one of the nucleic acids given in Table 1 and/or mentioned in the sequence protocol, or to a portion of any of the aforementioned nucleic acid sequences. Most preferably, the hybridizing sequence of a nucleic acid hybridises with a nucleic acid as represented by SEQ ID NO: 1 or SEQ ID NO: 50.

The term “hybridisation” as defined herein is a process wherein substantially homologous complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids. The stringency of hybridisation is influenced by conditions such as temperature, salt concentration, ionic strength and hybridisation buffer composition.

“Stringent hybridisation conditions” and “stringent hybridisation wash conditions” in the context of nucleic acid hybridisation experiments such as Southern and Northern hybridisations are sequence dependent and are different under different environmental parameters. The skilled artisan is aware of various parameters which may be altered during hybridisation and washing and which will either maintain or change the stringency conditions.

The Tₘ is the temperature under defined ionic strength and pH, at which 50% of the target sequence hybridises to a perfectly matched probe. The Tₘ is dependent upon the solution conditions and the base composition and length of the probe. For example, longer sequences hybridise specifically at higher temperatures. The maximum rate of hybridisation is obtained from about 16° C. up to 32° C. below Tₘ. The presence of monovalent cations in the hybridisation solution reduce the electrostatic repulsion between the two nucleic acid strands thereby promoting hybrid formation; this effect is visible for sodium concentrations of up to 0.4M. Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tₘ decreases about 1° C. per % base mismatch. The Tₘ may be calculated using the following equations, depending on the types of hybrids:

1. DNA-DNA hybrids (Meinkoth and Wahl, Anal. Biochem., 138: 267-284, 1984):

Tₘ=81.5° C.+16.6x log [Na⁺]^{a}+0.41x %[G/C^{b}]−500x[L^{c}]⁻¹−0.61x % formamide

2. DNA-RNA or RNA-RNA hybrids:

Tₘ=79.8+18.5(log₁₀ [Na⁺]^{a})+0.58(% G/C^{b})+11.8(% G/C^{b})²−820/L^{c}

3. oligo-DNA or oligo-RNA^{d} hybrids:
For <20 nucleotides: Tₘ=2 (lₙ)
For 20-35 nucleotides: Tₘ=22+1.46 (lₙ) ^{a} or for other monovalent cation, but only accurate in the 0.01-0.4 M range.^{b} only accurate for % GC in the 30% to 75% range.^{c} L=length of duplex in base pairs.^{d} Oligo, oligonucleotide; lₙ, effective length of primer=2 (no. of G/C)+(no. of A/T).

Note: for each 1% formamide, the Tₘ is reduced by about 0.6 to 0.7° C., while the presence of 6 M urea reduces the Tₘ by about 30° C.

Specificity of hybridisation is typically the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. Generally, suitable stringent conditions for nucleic acid hybridisation assays or gene amplification detection procedures are as set forth above. Conditions of greater or less stringency may also be selected. Generally, low stringency conditions are selected to be about 50° C. lower than the thermal melting point (Tₘ) for the specific sequence at a defined ionic strength and pH. Medium stringency conditions are when the temperature is 20° C. below Tₘ, and high stringency conditions are when the temperature is 10° C. below Tₘ. For example, stringent conditions are those that are at least as stringent as, for example, conditions A-L; and reduced stringency conditions are at least as stringent as, for example, conditions M-R. Non-specific binding may be controlled using any one of a number of known techniques such as, for example, blocking the membrane with protein-containing solutions, additions of heterologous RNA, DNA, and SDS to the hybridisation buffer, and treatment with RNase. Examples of hybridisation and wash conditions are listed in Table 2 below.

TABLE 2
Examples of hybridisation and wash conditions

| | | | | Wash |
|---|---|---|---|---|
| Stringency | Polynucleotide | Hybrid | Hybridization Temperature | Temperature |
| Condition | Hybrid^{±} | Length (bp)^{‡} | and Buffer^{†} | and Buffer^{†} |
| A | DNA:DNA | > or | 65° C. 1 SSC; or 42° C., | 65° C.; |
| | | equal to 50 | 1 SSC and 50% | 0.3 SSC |
| | | | formamide | |
| B | DNA:DNA | <50 | Tb*; 1 SSC | Tb*; 1 SSC |
| C | DNA:RNA | > or | 67° C. 1 SSC; or 45° C., | 67° C.; |
| | | equal to 50 | 1 SSC and 50% | 0.3 SSC |
| | | | formamide | |
| D | DNA:RNA | <50 | Td*; 1 SSC | Td*; 1 SSC |
| E | RNA:RNA | > or | 70° C. 1 SSC; or 50° C., | 70° C.; |
| | | equal to 50 | 1 SSC and 50% | 0.3 SSC |
| | | | formamide | |
| F | RNA:RNA | <50 | Tf*; 1 SSC | Tf*; 1 SSC |
| G | DNA:DNA | > or | 65° C. 4 SSC; or 45° C., | 65° C.; 1 SSC |
| | | equal to 50 | 4 SSC and 50% | |
| | | | formamide | |
| H | DNA:DNA | <50 | Th*; 4 SSC | Th*; 4 SSC |
| I | DNA:RNA | > or | 67° C. 4 SSC; or 45° C., | 67° C.; 1 SSC |
| | | equal to 50 | 4 SSC and 50% | |
| | | | formamide | |
| J | DNA:RNA | <50 | Tj*; 4 SSC | Tj*; 4 SSC |
| K | RNA:RNA | > or | 70° C. 4 SSC; or 40° C., | 67° C.; 1 SSC |
| | | equal to 50 | 6 SSC and 50% | |
| | | | formamide | |
| L | RNA:RNA | <50 | Tl*; 2 SSC | Tl*; 2 SSC |
| M | DNA:DNA | > or | 50° C. 4 SSC; or 40° C., | 50° C.; 2 SSC |
| | | equal to 50 | 6 SSC and 50% | |
| | | | formamide | |
| N | DNA:DNA | <50 | Tn*; 6 SSC | Tn*; 6 SSC |
| O | DNA:RNA | > or | 55° C. 4 SSC; or 42° C., | 55° C.; 2 SSC |
| | | equal to 50 | 6 SSC and 50% | |
| | | | formamide | |
| P | DNA:RNA | <50 | Tp*; 6 SSC | Tp*; 6 SSC |
| Q | RNA:RNA | > or | 60° C. 4 SSC; or 45° C., | 60° C.; |
| | | equal to 50 | 6 SSC and 50% | 2 SSC |
| | | | formamide | |
| R | RNA:RNA | <50 | Tr*; 4 SSC | Tr*; 4 SSC |

^{‡}The “hybrid length” is the anticipated length for the hybridising nucleic acid. When nucleic acids of known sequence are hybridised, the hybrid length may be determined by aligning the sequences and identifying the conserved regions described herein.
^{†}SSPE (1 SSPE is 0.15M NaCl, 10 mM NaH₂PO₄, and 1.25 mM EDTA, pH7.4) may be substituted for SSC (1 SSC is 0.15M NaCl and 15 mM sodium citrate) in the hybridisation and wash buffers; washes are performed for 15 minutes after hybridisation is complete. The hybridisations and washes may additionally include 5 Denhardt's reagent, 0.5-1.0% SDS, 100 μg/ml denatured, fragmented salmon sperm DNA, 0.5% sodium pyrophosphate, and up to 50% formamide.
*Tb-Tr: The hybridisation temperature for hybrids anticipated to be less than 50 base pairs in length should be 5-10° C. less than the melting temperature Tₘ of the hybrids; the Tₘ is determined according to the above-mentioned equations.
^{±}The present invention also encompasses the substitution of any one, or more DNA or RNA hybrid partners with either a PNA, or a modified nucleic acid.

For the purposes of defining the level of stringency, reference can be made to Sambrook et al. (2001) Molecular Cloning: a laboratory manual, 3^{rd} Edition Cold Spring Harbor Laboratory Press, CSH, New York or to Current Protocols in Molecular Biology, John Wiley & Sons, N.Y. (1989).

The two-WRKY domain nucleic acid or variant thereof may be derived from any natural or artificial source. The nucleic acid/gene or variant thereof may be isolated from a microbial source, such as yeast, fungi or slime mold, or from a plant, moss, algal or animal (including human) source. This nucleic acid may be modified from its native form in composition and/or genomic environment through deliberate human manipulation. The nucleic acid is preferably of plant origin, whether from the same plant species (for example to the one in which it is to be introduced) or whether from a different plant species. The nucleic acid may be isolated from a monocotyledonous species, preferably from the family Poaceae, further preferably from Oryza sativa or Zea mays. More preferably, the two-WRKY domain nucleic acid isolated from Oryza sativa or Zea mays is represented by SEQ ID NO: 1 or SEQ ID NO: 50, and the polypeptide sequence having two WRKY domains is as represented by SEQ ID NO: 2 or SEQ ID NO: 51.

The expression of a nucleic acid encoding a polypeptide having two WRKY domains or a homologue thereof may be modulated by introducing a genetic modification, within the locus of a two-WRKY domain gene, or elsewhere in the plant genome. The locus of a gene as defined herein is taken to mean a genomic region, which includes the gene of interest and 10 kb up- or downstream of the coding region.

The genetic modification may be introduced, for example, by any one (or more) of the following methods: T-DNA activation, TILLING, homologous recombination, site-directed mutagenesis, and directed evolution or by introducing and expressing in a plant a nucleic acid encoding a polypeptide having two WRKY domains or a homologue of such polypeptide. Following introduction of the genetic modification, there follows a step of selecting for modulated expression of a nucleic acid encoding a polypeptide having two WRKY domains or a homologue thereof, which modulation of expression gives plants having increased yield relative to control plants.

T-DNA activation tagging (Hayashi et al. Science (1992) 1350-1353) involves insertion of T-DNA, usually containing a promoter (may also be a translation enhancer or an intron), in the genomic region (locus) of the gene of interest or 10 kb up- or down stream of the coding region of a gene in a configuration such that the promoter directs expression of the targeted gene. Typically, regulation of expression of the targeted gene by its natural promoter is disrupted and the gene falls under the control of the newly introduced promoter. The promoter is typically embedded in a T-DNA. This T-DNA is randomly inserted into the plant genome, for example, through Agrobacterium infection and leads to overexpression of genes near the inserted T-DNA. The resulting transgenic plants show dominant phenotypes due to overexpression of genes close to the introduced promoter. The promoter to be introduced may be any promoter capable of directing expression of a gene in the desired organism, in this case a plant. For example, constitutive, tissue-preferred, cell type-preferred and inducible promoters are all suitable for use in T-DNA activation.

A genetic modification may also be introduced in the locus of a two-WRKY domain gene using the technique of TILLING (Targeted Induced Local Lesions In Genomes). This is a mutagenesis technology useful to generate and/or identify, and to eventually isolate mutagenised variants of a two-WRKY domain nucleic acid capable of exhibiting modulated WRKY activity. Mutant variants may also exhibit modified WRKY expression, in strength and in expression profile (time and place) than that exhibited by the gene in its natural form. TILLING also allows selection of plants carrying such mutant variants. TILLING combines high-density mutagenesis with high-throughput screening methods. The steps typically followed in TILLING are: (a) EMS mutagenesis (Redei G P and Koncz C (1992) In Methods in Arabidopsis Research, Koncz C, Chua N H, Schell J, eds. Singapore, World Scientific Publishing Co, pp. 16-82; Feldmann et al., (1994) In Meyerowitz E M, Somerville C R, eds, Arabidopsis. Cold Spring Harbor Laboratory Press, Cold Spring Harbor, N.Y., pp 137-172; Lightner J and Caspar T (1998) In J Martinez-Zapater, J Salinas, eds, Methods on Molecular Biology, Vol. 82. Humana Press, Totowa, N.J., pp 91-104); (b) DNA preparation and pooling of individuals; (c) PCR amplification of a region of interest; (d) denaturation and annealing to allow formation of heteroduplexes; (e) DHPLC, where the presence of a heteroduplex in a pool is detected as an extra peak in the chromatogram; (f) identification of the mutant individual; and (g) sequencing of the mutant PCR product. Methods for TILLING are well known in the art (McCallum et al., (2000) Nat Biotechnol 18: 455-457; reviewed by Stemple (2004) Nat Rev Genet 5(2): 145-50).

Homologous recombination allows introduction in a genome of a selected nucleic acid at a defined selected position. Homologous recombination is a standard technology used routinely in biological sciences for lower organisms such as yeast or the moss Physcomitrella. Methods for performing homologous recombination in plants have been described not only for model plants (Offring a et al. (1990) EMBO J 9(10): 3077-84) but also for crop plants, for example rice (Terada et al. (2002) Nat Biotech 20(10): 1030-4; lida and Terada (2004) Curr Opin Biotech 15(2): 132-8). The nucleic acid to be targeted (which may be a two-WRKY domain nucleic acid or variant thereof as hereinbefore defined) need not be targeted to the locus of a two-WRKY domain gene, but may be introduced in, for example, regions of high expression. The nucleic acid to be targeted may be an improved allele used to replace the endogenous gene or may be introduced in addition to the endogenous gene.

Site-directed mutagenesis may be used to generate variants of two-WRKY domain nucleic acids. Several methods are available to achieve site-directed mutagenesis, the most common being PCR based methods (Current Protocols in Molecular Biology. Wiley Eds. http://www.4ulr.com/products/currentprotocols/index.html).

Directed evolution may also be used to generate variants of two-WRKY domain nucleic acids. This consists of iterations of DNA shuffling followed by appropriate screening and/or selection to generate variants of two-WRKY domain nucleic acids or portions thereof encoding polypeptides having two WRKY domains or homologues or portions thereof having a modified biological activity [Castle et al., (2004) Science 304(5674): 1151-4; U.S. Pat. Nos. 5,811,238 and 6,395,547].

T-DNA activation, TILLING, homologous recombination, site-directed mutagenesis and directed evolution are examples of technologies that enable the generation of novel alleles and variants of two-WRKY domain nucleic acids.

A preferred method for introducing a genetic modification (which in this case need not be in the locus of a two-WRKY domain gene) is to introduce and express in a plant a nucleic acid encoding a polypeptide having two WRKY domains or a homologue of such polypeptide.

“Homologues” of a polypeptide having two WRKY domains may also be useful in the present invention. Homologues encompass peptides, oligopeptides, polypeptides, proteins and enzymes having amino acid substitutions, deletions and/or insertions relative to the unmodified protein in question and having similar biological and functional activity as the unmodified protein from which they are derived. To produce such homologues, amino acids of the protein may be replaced by other amino acids having similar properties (such as similar hydrophobicity, hydrophilicity, antigenicity, propensity to form or break α-helical structures or β-sheet structures). Conservative substitution tables are well known in the art (see for example Creighton (1984) Proteins. W.H. Freeman and Company and Table 3 below).

Also encompassed by the term “homologues” are two special forms of homology, which include orthologous sequences and paralogous sequences, which encompass evolutionary concepts used to describe ancestral relationships of genes. The term “paralogous” relates to gene-duplications within the genome of a species leading to paralogous genes. The term “orthologous” relates to homologous genes in different organisms due to speciation. Examples of homologues of a polypeptide having two WRKY domains are given in Table 1 or in the sequence protocol hereinabove.

Orthologues in, for example, monocot plant species may easily be found by performing a so-called reciprocal blast search. This may be done by a first BLAST involving BLASTing a query sequence (for example, SEQ ID NO: 1, SEQ ID NO: 50, SEQ ID NO: 2 or SEQ ID NO: 51) against any sequence database, such as the publicly available NCBI database. BLASTN or TBLASTX (using standard default values) may be used when starting from a nucleotide sequence and BLASTP or TBLASTN (using standard default values) may be used when starting from a polypeptide sequence. The BLAST results may optionally be filtered. The full-length sequences of either the filtered results or non-filtered results are then BLASTed back (second BLAST) against sequences from the organism from which the query sequence is derived (where the query sequence is SEQ ID NO: 1, SEQ ID NO: 50, SEQ ID NO: 2 or SEQ ID NO: 51, the second BLAST would therefore be against Oryza or Zea sequences). The results of the first and second BLASTs are then compared. A paralogue is identified if a high-ranking hit from the first BLAST is from the same species as from which the query sequence is derived, a BLAST back then ideally results in the query sequence as high-ranking hit (besides the paralogue sequence itself); an orthologue is identified if a high-ranking hit in the first BLAST is not from the same species as from which the query sequence is derived and preferably results upon BLAST back in the query sequence amongst the highest hits. High-ranking hits are those having a low E-value. The lower the E-value, the more significant the score (or in other words the lower the chance that the hit was found by chance). Computation of the E-value is well known in the art. In addition to E-values, comparisons are also scored by percentage identity. Percentage identity refers to the number of identical nucleotides (or amino acids) between the two compared nucleic acid (or polypeptide) sequences over a particular length. In the case of large families, ClustalW may be used, followed by a neighbour joining tree, to help visualize the clustering.

A homologue may be in the form of a “substitutional variant” of a protein, i.e. where at least one residue in an amino acid sequence has been removed and a different residue inserted in its place. Amino acid substitutions are typically of single residues, but may be clustered depending upon functional constraints placed upon the polypeptide; insertions will usually be of the order of about 1 to 10 amino acid residues. Preferably, amino acid substitutions comprise conservative amino acid substitutions. Conservative substitution tables are readily available in the art. The table below gives examples of conserved amino acid substitutions.

TABLE 3
Examples of conserved amino acid substitutions

| | Conservative | | Conservative |
|---|---|---|---|
| Residue | Substitutions | Residue | Substitutions |
| Ala | Ser | Leu | Ile; Val |
| Arg | Lys | Lys | Arg; Gln |
| Asn | Gln; His | Met | Leu; Ile |
| Asp | Glu | Phe | Met; Leu; Tyr |
| Gln | Asn | Ser | Thr; Gly |
| Cys | Ser | Thr | Ser; Val |
| Glu | Asp | Trp | Tyr |
| Gly | Pro | Tyr | Trp; Phe |
| His | Asn; Gln | Val | Ile; Leu |
| Ile | Leu, Val | | |

A homologue may also be in the form of an “insertional variant” of a protein, i.e. where one or more amino acid residues are introduced into a predetermined site in a protein. Insertions may comprise N-terminal and/or C-terminal fusions as well as intra-sequence insertions of single or multiple amino acids. Generally, insertions within the amino acid sequence will be smaller than N- or C-terminal fusions, of the order of about 1 to 10 residues. Examples of N- or C-terminal fusion proteins or peptides include the binding domain or activation domain of a transcriptional activator as used in the yeast two-hybrid system, phage coat proteins, (histidine)-6-tag, glutathione S-transferase-tag, protein A, maltose-binding protein, dihydrofolate reductase, Tag•100 epitope, c-myc epitope, FLAG®-epitope, lacZ, CMP (calmodulin-binding peptide), HA epitope, protein C epitope and VSV epitope.

Homologues in the form of “deletion variants” of a protein are characterised by the removal of one or more amino acids from a protein.

Amino acid variants of a protein may readily be made using peptide synthetic techniques well known in the art, such as solid phase peptide synthesis and the like, or by recombinant DNA manipulations. Methods for the manipulation of DNA sequences to produce substitution, insertion or deletion variants of a protein are well known in the art. For example, techniques for making substitution mutations at predetermined sites in DNA are well known to those skilled in the art and include M13 mutagenesis, T7-Gen in vitro mutagenesis (USB, Cleveland, Ohio), QuickChange Site-Directed mutagenesis (Stratagene, San Diego, Calif.), PCR-mediated site-directed mutagenesis or other site-directed mutagenesis protocols.

The polypeptide having two WRKY domains or homologue thereof may be a derivative. “Derivatives” include peptides, oligopeptides, polypeptides, proteins and enzymes which may comprise substitutions, deletions or additions of naturally and non-naturally occurring amino acid residues compared to the amino acid sequence of a naturally-occurring form of the protein. “Derivatives” of a protein encompass peptides, oligopeptides, polypeptides, proteins and enzymes which may comprise naturally occurring altered, glycosylated, acylated, prenylated, sumoylated or non-naturally occurring amino acid residues compared to the amino acid sequence of a naturally-occurring form of the polypeptide. A derivative may also comprise one or more non-amino acid substituents compared to the amino acid sequence from which it is derived, for example a reporter molecule or other ligand, covalently or non-covalently bound to the amino acid sequence, such as a reporter molecule which is bound to facilitate its detection, and non-naturally occurring amino acid residues relative to the amino acid sequence of a naturally-occurring protein.

The polypeptide having two WRKY domains or homologue thereof may be encoded by an alternative splice variant of a two-WRKY domain nucleic acid/gene. The term “alternative splice variant” as used herein encompasses variants of a nucleic acid sequence in which selected introns and/or exons have been excised, replaced or added, or in which introns have been shortened or lengthened. Such variants will be ones in which the biological activity of the protein is retained, which may be achieved by selectively retaining functional segments of the protein. Such splice variants may be found in nature or may be manmade. Methods for making such splice variants are well known in the art. The splice variant may include the nucleotides encoding a polypeptide comprising from amino-terminus to carboxy-terminus: (i) a Pro-Ser rich domain, and (ii) two WRKY domains including a zinc-finger C₂—H₂ motif. The splice variant may optionally comprise any one or more of the following: (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39. The splice may further comprise an LXSP motif within the Pro-Ser rich domain (where L is Leu, S is Ser, P is Pro and X is any amino acid). Preferred splice variants are splice variants of a nucleic acid encoding a polypeptide having two WRKY domains as represented any one of the nucleic acids given in Table 1 and/or in the sequence protocol. Most preferred is a splice variant of a nucleic acid as represented by SEQ ID NO: 1 or SEQ ID NO: 50.

The homologue may also be encoded by an allelic variant of a nucleic acid encoding a polypeptide having two WRKY domains or a homologue thereof. Allelic variants exist in nature, and encompassed within the methods of the present invention is the use of these natural alleles. Allelic variants encompass Single Nucleotide Polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms. The allelic variant may include the nucleotides encoding a polypeptide comprising from amino-terminus to carboxy-terminus: (i) a Pro-Ser rich domain, and (ii) two WRKY domains including a zinc-finger C2-H2 motif. The allelic variant may optionally comprise any one or more of the following: (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); and (iii) a conserved domain with at least 50%, 60% or 70%, preferably 75% or 80%, more preferably 90%, even more preferably 91%, 92%, 93%, 94% or 95%, most preferably 96%, 97%, 98% or 99% identity to SEQ ID NO: 39. The allelic variant may further comprise an LXSP motif within the Pro-Ser rich domain (where L is Leu, S is Ser, P is Pro and X is any amino acid). Preferred allelic variants are allelic variants of a nucleic acid encoding a polypeptide having two WRKY domains as represented any one of the nucleic acids given in Table 1 and/or in the sequence protocol. Most preferred are an allelic variant of a nucleic acid as represented by SEQ ID NO: 1 or SEQ ID NO: 50.

Splice variants and allelic variants of nucleic acids encoding a polypeptide having two WRKY domains are examples of nucleic acids useful in performing the methods of the invention.

According to a preferred aspect of the present invention, modulated expression of the two-WRKY domain nucleic acid or variant thereof is envisaged. Methods for increasing expression of genes or gene products are well documented in the art and include, for example, overexpression driven by appropriate promoters, the use of transcription enhancers or translation enhancers. Isolated nucleic acids which serve as promoter or enhancer elements may be introduced in an appropriate position (typically upstream) of a non-heterologous form of a polynucleotide so as to upregulate expression of a two-WRKY domain nucleic acid or variant thereof. For example, endogenous promoters may be altered in vivo by mutation, deletion, and/or substitution (see, Kmiec, U.S. Pat. No. 5,565,350; Zarling et al., PCT/US93/03868), or isolated promoters may be introduced into a plant cell in the proper orientation and distance from a gene of the present invention so as to control the expression of the gene. Methods for reducing the expression of genes or gene products are well documented in the art and include, for example, downregulation of expression by anti-sense techniques, cosuppression, RNAi techniques (using hairpin RNAs (hpRNAs), small interference RNAs (siRNAs), microRNA (miRNA)) etc.

If polypeptide expression is desired, it is generally desirable to include a polyadenylation region at the 3′-end of a polynucleotide coding region. The polyadenylation region can be derived from the natural gene, from a variety of other plant genes, or from T-DNA. The 3′ end sequence to be added may be derived from, for example, the nopaline synthase or octopine synthase genes, or alternatively from another plant gene, or less preferably from any other eukaryotic gene.

An intron sequence may also be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg (1988) Mol. Cell biol. 8:4395-4405; Callis et al. (1987) Genes Dev. 1:1183-1200). Such intron enhancement of gene expression is typically greatest when placed near the 5′ end of the transcription unit. Use of the maize introns Adh1-S intron 1, 2, and 6, the Bronze-1 intron are known in the art. See generally, The Maize Handbook, Chapter 116, Freeling and Walbot, Eds., Springer, N.Y. (1994).

The invention also provides genetic constructs and vectors to facilitate introduction and/or expression of the nucleotide sequences useful in the methods according to the invention.

Therefore, there is provided a gene construct comprising:
(i) a two-WRKY domain nucleic acid or variant thereof, as defined hereinabove;
(ii) one or more control sequences capable of driving expression of the nucleic acid sequence of (i); and optionally
(iii) a transcription termination sequence.

Constructs useful in the methods according to the present invention may be constructed using recombinant DNA technology well known to persons skilled in the art. The gene constructs may be inserted into vectors, which may be commercially available, suitable for transforming into plants and suitable for expression of the gene of interest in the transformed cells. The invention therefore provides use of a gene construct as defined hereinabove in the methods of the invention.

Plants are transformed with a vector comprising the sequence of interest (i.e., a nucleic acid encoding a polypeptide having two WRKY domains or homologue of such polypeptide). The sequence of interest is operably linked to one or more control sequences (at least to a promoter). The terms “regulatory element”, “control sequence” and “promoter” are all used interchangeably herein and are to be taken in a broad context to refer to regulatory nucleic acid sequences capable of effecting expression of the sequences to which they are ligated. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences. The term “regulatory element” also encompasses a synthetic fusion molecule or derivative that confers, activates or enhances expression of a nucleic acid molecule in a cell, tissue or organ. The term “operably linked” as used herein refers to a functional linkage between the promoter sequence and the gene of interest, such that the promoter sequence is able to initiate transcription of the gene of interest.

Suitable promoters, which are functional in plants, are generally known. They may take the form of constitutive or inducible promoters. Suitable promoters can enable the development- and/or tissue-specific expression in multi-celled eukaryotes; thus, leaf-, root-, flower-, seed-, stomata-, tuber- or fruit-specific promoters may advantageously be used in plants.

Different plant promoters usable in plants are promoters such as, for example, the USP, the LegB4-, the DC3 promoter or the ubiquitin promoter from parsley.

A “plant” promoter comprises regulatory elements, which mediate the expression of a coding sequence segment in plant cells. Accordingly, a plant promoter need not be of plant origin, but may originate from viruses or microorganisms, in particular for example from viruses which attack plant cells.

The “plant” promoter can also originates from a plant cell, e.g. from the plant, which is transformed with the nucleic acid sequence to be expressed in the inventive process and described herein. This also applies to other “plant” regulatory signals, for example in “plant” terminators.

For expression in plants, the nucleic acid molecule must, as described above, be linked operably to or comprise a suitable promoter which expresses the gene at the right point in time and in a cell- or tissue-specific manner. Usable promoters are constitutive promoters (Benfey et al., EMBO J. 8 (1989) 2195-2202), such as those which originate from plant viruses, such as 35S CAMV (Franck et al., Cell 21 (1980) 285-294), 19S CaMV (see also U.S. Pat. No. 5,352,605 and WO 84/02913), 34S FMV (Sanger et al., Plant. Mol. Biol., 14, 1990: 433-443), or plant promoters such as the parsley ubiquitin promoter, the Rubisco small subunit promoter described in U.S. Pat. No. 4,962,028 or the plant promoters PRP1 [Ward et al., Plant. Mol. Biol. 22 (1993)], SSU, PGEL1, OCS [Leisner (1988) Proc Natl Acad Sci USA 85(5): 2553-2557], lib4, usp, mas [Comai (1990) Plant Mol Biol 15 (3):373-381], STLS1, ScBV (Schenk (1999) Plant Mol Biol 39(6):1221-1230), B33, SAD1 or SAD2 (flax promoters, Jain et al., Crop Science, 39 (6), 1999: 1696-1701) or nos [Shaw et al. (1984) Nucleic Acids Res. 12(20):7831-7846]. Further examples of constitutive plant promoters are the sugarbeet V-ATPase promoters (WO 01/14572). Examples of synthetic constitutive promoters are the Super promoter (WO 95/14098) and promoters derived from G-boxes (WO 94/12015). If appropriate, chemical inducible promoters may furthermore also be used, compare EP-A 388186, EP-A 335528, WO 97/06268. Stable, constitutive expression of the proteins according to the invention a plant can be advantageous. However, inducible expression of the polypeptide of the invention is advantageous, if a late expression before the harvest is of advantage, as metabolic manipulation may lead to plant growth retardation.

The expression of plant genes can also be facilitated via a chemical inducible promoter (for a review, see Gatz 1997, Annu. Rev. Plant Physiol. Plant Mol. Biol., 48:89-108). Chemically inducible promoters are particularly suitable when it is desired to express the gene in a time-specific manner. Examples of such promoters are a salicylic acid inducible promoter (WO 95/19443), and abscisic acid-inducible promoter (EP 335 528), a tetracyclin-inducible promoter (Gatz et al. (1992) Plant J. 2, 397-404), a cyclohexanol- or ethanol-inducible promoter (WO 93/21334) or others as described herein.

Other suitable promoters are those which react to biotic or abiotic stress conditions, for example the pathogen-induced PRP1 gene promoter (Ward et al., Plant. Mol. Biol. 22 (1993) 361-366), the tomato heat-inducible hsp80 promoter (U.S. Pat. No. 5,187,267), the potato chill-inducible alpha-amylase promoter (WO 96/12814) or the wound-inducible pinII promoter (EP-A-0 375 091) or others as described herein.

Preferred promoters are in particular those which bring gene expression in tissues and organs, in seed cells, such as endosperm cells and cells of the developing embryo. Suitable promoters are the oilseed rape napin gene promoter (U.S. Pat. No. 5,608,152), the Vicia faba USP promoter (Baeumlein et al., Mol Gen Genet, 1991, 225 (3): 459-67), the Arabidopsis oleosin promoter (WO 98/45461), the Phaseolus vulgaris phaseolin promoter (U.S. Pat. No. 5,504,200), the Brassica Bce4 promoter (WO 91/13980), the bean arc5 promoter, the carrot DcG3 promoter, or the Legumin B4 promoter (LeB4; Baeumlein et al., 1992, Plant Journal, 2 (2): 233-9), and promoters which bring about the seed-specific expression in monocotyledonous plants such as maize, barley, wheat, rye, rice and the like. Advantageous seed-specific promoters are the sucrose binding protein promoter (WO 00/26388), the phaseolin promoter and the napin promoter. Suitable promoters which must be considered are the barley lpt2 or lpt1 gene promoter (WO 95/15389 and WO 95/23230), and the promoters described in WO 99/16890 (promoters from the barley hordein gene, the rice glutelin gene, the rice oryzin gene, the rice prolamin gene, the wheat gliadin gene, the wheat glutelin gene, the maize zein gene, the oat glutelin gene, the sorghum kasirin gene and the rye secalin gene). Further suitable promoters are Amy32b, Amy 6-6 and Aleurain [U.S. Pat. No. 5,677,474], Bce4 (oilseed rape) [U.S. Pat. No. 5,530,149], glycinin (soya) [EP 571 741], phosphoenolpyruvate carboxylase (soya) [JP 06/62870], ADR12-2 (soya) [WO 98/08962], isocitrate lyase (oilseed rape) [U.S. Pat. No. 5,689,040] or α-amylase (barley) [EP 781 849]. Other promoters which are available for the expression of genes in plants are leaf-specific promoters such as those described in DE-A 19644478 or light-regulated promoters such as, for example, the pea petE promoter.

Further suitable plant promoters are the cytosolic FBPase promoter or the potato ST-LSI promoter (Stockhaus et al., EMBO J. 8, 1989, 2445), the Glycine max phosphoribosylpyrophosphate amidotransferase promoter (GenBank Accession No. U87999) or the node-specific promoter described in EP-A0 249 676.

Advantageously, any type of promoter may be used to drive expression of the nucleic acid sequence. The promoter may be an inducible promoter, i.e. having induced or increased transcription initiation in response to a developmental, chemical, environmental or physical stimulus. An example of an inducible promoter being a stress-inducible promoter, i.e. a promoter activated when a plant is exposed to various stress conditions. Additionally or alternatively, the promoter may be a tissue-specific promoter, i.e. one that is capable of preferentially initiating transcription in certain tissues, such as the leaves, roots, seed tissue etc.

In one embodiment, the two-WRKY domain nucleic acid or variant thereof is operably linked to a constitutive promoter. A constitutive promoter is transcriptionally active during most, but not necessarily all, phases of its growth and development and is substantially ubiquitously expressed. Preferably, the constitutive promoter is a GOS2 promoter (from rice) (SEQ ID NO: 42). Examples of other constitutive promoters that may also be used to drive expression of a two-WRKY domain nucleic acid are shown in Table 4 below.

TABLE 4
Examples of constitutive promoters

| | Expression | |
|---|---|---|
| Gene Source | Motif | Reference |
| Actin | Constitutive | McElroy et al, Plant Cell, 2: 163-171, |
| | | 1990 |
| CAMV 35S | Constitutive | Odell et al, Nature, 313: 810-812, |
| | | 1985 |
| CaMV 19S | Constitutive | Nilsson et al., Physiol. Plant. |
| | | 100: 456-462, 1997 |
| GOS2 | Constitutive | de Pater et al, Plant J |
| | | Nov; 2(6): 837-44, 1992 |
| Ubiquitin | Constitutive | Christensen et al, Plant Mol. Biol. |
| | | 18: 675-689, 1992 |
| Rice cyclophilin | Constitutive | Buchholz et al, Plant Mol Biol. |
| | | 25(5): 837-43, 1994 |
| Maize H3 histone | Constitutive | Lepetit et al, Mol. Gen. Genet. |
| | | 231: 276-285, 1992 |
| Actin 2 | Constitutive | An et al, Plant J. 10(1); 107-121, |
| | | 1996 |

In another embodiment, the two-WRKY domain nucleic acid or variant thereof is operably linked to a seed-specific promoter, preferably an embryo and/or aleurone specific promoter. Preferably, the embryo and/or aleurone specific promoter is an oleosin promoter, more preferably the embryo and/or aleurone specific promoter is an 18 kDa oleosin promoter, further preferably the embryo and/or aleurone specific promoter is a rice 18 kDa oleosin promoter (Wu et al. (1998) J Biochem 123(3): 386-91), most preferably the embryo and/or aleurone specific promoter is substantially similar to the sequence as represented by SEQ ID NO: 43 or is as represented by SEQ ID NO: 43. Examples of other seed-specific promoters that may also be used to drive expression of a two-WRKY domain nucleic acid are shown in Table 5 below.

TABLE 5
Examples of seed-specific promoters

| Gene source | | |
|---|---|---|
| and name | Expression Pattern | Reference |
| Rice RP6 | Endosperm-specific | Wen et al. (1993) Plant Physiol |
| | | 101(3): 1115-6 |
| Sorghum kafirin | Endosperm-specific | DeRose et al. (1996) Plant |
| | | Molec Biol 32: 1029-35 |
| Corn zein | Endosperm-specific | Matzke et al. (1990) Plant Mol |
| | | Biol 14(3): 323-32 |
| Rice Oleosin | Embryo (and | Chuang et al. (1996) J Biochem |
| 18 kDa | aleurone) specific | 120(1): 74-81 |
| Rice Oleosin | Embryo (and | Chuang et al. (1996) J Biochem |
| 16 kDa | aleurone) specific | 120(1): 74-81 |
| Soybean beta- | Embryo | Chiera et al. (2005) Plant Molec |
| conglycinin | | Biol 56(6): 895-904 |
| Rice Wsi18 | Whole seed | Joshee et al. (1998) Plant Cell |
| | | Physiol 39(1): 64-72. |
| Rice | Whole seed | Sasaki et al. (2002) NCBI |
| | | accession number BAA85411 |

It should be clear that the applicability of the present invention is not restricted to the two-WRKY domain nucleic acid represented by SEQ ID NO: 1 or SEQ ID NO: 50, nor is the applicability of the invention restricted to expression of a two-WRKY domain nucleic acid when driven by either a GOS2 promoter or an oleosin promoter.

Optionally, one or more terminator sequences may also be used in the construct introduced into a plant. The term “terminator” encompasses a control sequence which is a DNA sequence at the end of a transcriptional unit which signals 3′ processing and polyadenylation of a primary transcript and termination of transcription. Additional regulatory elements may include transcriptional as well as translational enhancers. Those skilled in the art will be aware of terminator and enhancer sequences that may be suitable for use in performing the invention. Such sequences would be known or may readily be obtained by a person skilled in the art.

The genetic constructs of the invention may further include an origin of replication sequence that is required for maintenance and/or replication in a specific cell type. One example is when a genetic construct is required to be maintained in a bacterial cell as an episomal genetic element (e.g. plasmid or cosmid molecule). Preferred origins of replication include, but are not limited to, the f1-ori and colE1.

For the detection and/or selection of the successful transfer of the nucleic acid sequences as depicted in the sequence protocol and used in the process of the invention, it is advantageous to use marker genes (=reporter genes). These marker genes enable the identification of a successful transfer of the nucleic acid molecules via a series of different principles, for example via visual identification with the aid of fluorescence, luminescence or in the wavelength range of light which is discernible for the human eye, by a resistance to herbicides or antibiotics, via what are known as nutritive markers (auxotrophism markers) or antinutritive markers, via enzyme assays or via phytohormones. Examples of such markers which may be mentioned are GFP (=green fluorescent protein); the luciferin/luceferase system, the β-galactosidase with its colored substrates, for example X-Gal, the herbicide resistances to, for example, imidazolinone, glyphosate, phosphinothricin or sulfonylurea, the antibiotic resistances to, for example, bleomycin, hygromycin, streptomycin, kanamycin, tetracyclin, chloramphenicol, ampicillin, gentamycin, geneticin (G418), spectinomycin or blasticidin, to mention only a few, nutritive markers such as the utilization of mannose or xylose, or antinutritive markers such as the resistance to 2-deoxyglucose. This list is a small number of possible markers. The skilled worker is very familiar with such markers. Different markers are preferred, depending on the organism and the selection method.

Therefore genetic construct may optionally comprise a selectable marker gene. As used herein, the term “selectable marker or selectable marker gene” includes any gene that confers a phenotype on a cell in which it is expressed to facilitate the identification and/or selection of cells that are transfected or transformed with a nucleic acid construct of the invention. Suitable markers may be selected from markers that confer antibiotic or herbicide resistance, that introduce a new metabolic trait or that allow visual selection. Examples of selectable marker genes include genes conferring resistance to antibiotics (such as nptII that phosphorylates neomycin and kanamycin, or hpt, phosphorylating hygromycin), to herbicides (for example bar which provides resistance to Basta; aroA or gox providing resistance against glyphosate), or genes that provide a metabolic trait (such as manA that allows plants to use mannose as sole carbon source). Visual marker genes result in the formation of colour (for example β-glucuronidase, GUS), luminescence (such as luciferase) or fluorescence (Green Fluorescent Protein, GFP, and derivatives thereof).

It is known of the stable or transient integration of nucleic acids into plant cells that only a minority of the cells takes up the foreign DNA and, if desired, integrates it into its genome, depending on the expression vector used and the transfection technique used. To identify and select these integrants, a gene encoding for a selectable marker (as described above, for example resistance to antibiotics) is usually introduced into the host cells together with the gene of interest. Preferred selectable markers in plants comprise those, which confer resistance to an herbicide such as glyphosate or gluphosinate. Other suitable markers are, for example, markers, which encode genes involved in biosynthetic pathways of, for example, sugars or amino acids, such as β-galactosidase, ura3 or ilv2. Markers, which encode genes such as luciferase, gfp or other fluorescence genes, are likewise suitable. These markers and the aforementioned markers can be used in mutants in whom these genes are not functional since, for example, they have been deleted by conventional methods. Furthermore, nucleic acid molecules, which encode a selectable marker, can be introduced into a host cell on the same vector as those, which encode the polypeptides of the invention or used in the process or else in a separate vector. Cells which have been transfected stably with the nucleic acid introduced can be identified for example by selection (for example, cells which have integrated the selectable marker survive whereas the other cells die).

Since the marker genes, as a rule specifically the gene for resistance to antibiotics and herbicides, are no longer required or are undesired in the transgenic host cell once the nucleic acids have been introduced successfully, the process according to the invention for introducing the nucleic acids advantageously employs techniques which enable the removal, or excision, of these marker genes. One such a method is what is known as cotransformation. The cotransformation method employs two vectors simultaneously for the transformation, one vector bearing the nucleic acid according to the invention and a second bearing the marker gene(s). A large proportion of transformants receives or, in the case of plants, comprises (up to 40% of the transformants and above), both vectors. In case of transformation with Agrobacteria, the transformants usually receive only a part of the vector, the sequence flanked by the T-DNA, which usually represents the expression cassette. The marker genes can subsequently be removed from the transformed plant by performing crosses. In another method, marker genes integrated into a transposon are used for the transformation together with desired nucleic acid (known as the Ac/Ds technology). The transformants can be crossed with a transposase resource or the transformants are transformed with a nucleic acid construct conferring expression of a transposase, transiently or stable. In some cases (approx. 10%), the transposon jumps out of the genome of the host cell once transformation has taken place successfully and is lost. In a further number of cases, the transposon jumps to a different location. In these cases, the marker gene must be eliminated by performing crosses. In microbiology, techniques were developed which make possible, or facilitate, the detection of such events. A further advantageous method relies on what are known as recombination systems; whose advantage is that elimination by crossing can be dispensed with. The best-known system of this type is what is known as the Cre/lox system. Cre1 is a recombinase, which removes the sequences located between the loxP sequences. If the marker gene is integrated between the loxP sequences, it is removed, once transformation has taken place successfully, by expression of the recombinase. Further recombination systems are the HIN/HIX, FLP/FRT and REP/STB system (Tribble et al., J. Biol. Chem., 275, 2000: 22255-22267; Velmurugan et al., J. Cell Biol., 149, 2000: 553-566). A site-specific integration into the plant genome of the nucleic acid sequences according to the invention is possible. Naturally, these methods can also be applied to microorganisms such as yeast, fungi or bacteria.

The present invention also encompasses plants obtainable by the methods according to the present invention. The present invention therefore provides plants, plant parts (including seed) and plant cells obtainable by the method according to the present invention, which plants, plant parts and plant cells have introduced therein a two-WRKY domain nucleic acid or variant thereof.

The invention also provides a method for the production of transgenic plants having increased yield relative to control plants, comprising introduction and expression in a plant of a two-WRKY domain nucleic acid or a variant thereof.

More specifically, the present invention provides a method for the production of transgenic plants having increased yield relative to control plants, which method comprises:
(i) introducing and expressing in a plant or plant cell a two-WRKY domain nucleic acid or variant thereof as defined herein; and
(ii) cultivating the plant cell under conditions promoting plant growth and development.

The nucleic acid may be introduced directly into a plant cell or into the plant itself (including introduction into a tissue, organ or any other part of a plant). According to a preferred feature of the present invention, the nucleic acid is preferably introduced into a plant by transformation.

The term “introduction” or “transformation” as referred to herein encompasses the transfer of an exogenous polynucleotide into a host cell, irrespective of the method used for transfer. Plant tissue capable of subsequent clonal propagation, whether by organogenesis or embryogenesis, may be transformed with a genetic construct of the present invention and a whole plant regenerated therefrom. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art.

The transfer of foreign genes into the genome of a plant is called transformation. In doing this the methods described for the transformation and regeneration of plants from plant tissues or plant cells are utilized for transient or stable transformation. An advantageous transformation method is the transformation in planta. To this end, it is possible, for example, to allow the agrobacteria to act on plant seeds or to inoculate the plant meristem with agrobacteria. It has proved particularly expedient in accordance with the invention to allow a suspension of transformed agrobacteria to act on the intact plant or at least the flower primordia. The plant is subsequently grown on until the seeds of the treated plant are obtained (Clough and Bent, Plant J. (1998) 16, 735743). To select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Further advantageous transformation methods, in particular for plants, are known to the skilled worker and are described herein below.

Transformation of plant species is now a fairly routine technique. Advantageously, any of several transformation methods may be used to introduce the gene of interest into a suitable ancestor cell. Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al., 1985 Bio/Technol 3, 1099-1102); microinjection into plant material (Crossway A et al., (1986) Mol. Gen Genet 202: 179-185); DNA or RNA-coated particle bombardment (Klein T M et al., (1987) Nature 327: 70) infection with (non-integrative) viruses and the like. Transgenic rice plants expressing a two-WRKY domain nucleic acid/gene are preferably produced via Agrobacterium-mediated transformation using any of the well known methods for rice or corn transformation, such as described in any of the following: published European patent application EP 1198985 A1, Aldemita and Hodges (Planta 199: 612-617, 1996); Chan et al. (Plant Mol Biol 22 (3): 491-506, 1993), Hiei et al. (Plant J 6 (2): 271-282, 1994), which disclosures are incorporated by reference herein as if fully set forth. In the case of corn transformation, the preferred method is as described in either Ishida et al. (Nat. Biotechnol 14(6): 745-50, 1996) or Frame et al. (Plant Physiol 129(1): 13-22, 2002), which disclosures are incorporated by reference herein as if fully set forth. Said methods are further described by way of example in B. Jenes et al., Techniques for Gene Transfer, in: Transgenic Plants, Vol. 1, Engineering and Utilization, eds. S. D. Kung and R. Wu, Academic Press (1993) 128-143 and in Potrykus Annu. Rev. Plant Physiol. Plant Molec. Biol. 42 (1991) 205-225). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, in particular of crop plants such as by way of example tobacco plants, for example by bathing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

Generally after transformation, plant cells or cell groupings are selected for the presence of one or more markers which are encoded by plant-expressible genes co-transferred with the gene of interest, following which the transformed material is regenerated into a whole plant.

As mentioned Agrobacteria transformed with an expression vector according to the invention may also be used in the manner known per se for the transformation of plants such as experimental plants like Arabidopsis or crop plants, such as, for example, cereals, maize, oats, rye, barley, wheat, soya, rice, cotton, sugarbeet, canola, sunflower, flax, hemp, potato, tobacco, tomato, carrot, bell peppers, oilseed rape, tapioca, cassaya, arrow root, tagetes, alfalfa, lettuce and the various tree, nut, and grapevine species, in particular oil-containing crop plants such as soya, peanut, castor-oil plant, sunflower, maize, cotton, flax, oilseed rape, coconut, oil palm, safflower (Carthamus tinctorius) or cocoa beans, for example by bathing scarified leaves or leaf segments in an agrobacterial solution and subsequently growing them in suitable media.

In addition to the transformation of somatic cells, which then has to be regenerated into intact plants, it is also possible to transform the cells of plant meristems and in particular those cells which develop into gametes. In this case, the transformed gametes follow the natural plant development, giving rise to transgenic plants. Thus, for example, seeds of Arabidopsis are treated with agrobacteria and seeds are obtained from the developing plants of which a certain proportion is transformed and thus transgenic [Feldman, K A and Marks M D (1987). Mol Gen Genet 208:274-289; Feldmann K (1992). In: C Koncz, N-H Chua and J Shell, eds, Methods in Arabidopsis Research. Word Scientific, Singapore, pp. 274-289]. Alternative methods are based on the repeated removal of the influorescences and incubation of the excision site in the center of the rosette with transformed agrobacteria, whereby transformed seeds can likewise be obtained at a later Two-WRKY domain nucleic acids or variants thereof, or polypeptides having two WRKY domains or homologues thereof may find use in breeding programmes in which a DNA marker is identified which may be genetically linked to a two-WRKY domain gene or variant thereof. The two-WRKY domain nucleic acids/genes or variants thereof, or polypeptides having two WRKY domains or homologues thereof may be used to define a molecular marker. This DNA or protein marker may then be used in breeding programmes to select plants having increased yield. The two-WRKY domain gene or variant thereof may, for example, be a nucleic acid as represented by any one of the nucleic acids given in Table 1 and/or in the sequence protocol.

Allelic variants of a two-WRKY domain nucleic acid/gene may also find use in marker-assisted breeding programmes. Such breeding programmes sometimes require introduction of allelic variation by mutagenic treatment of the plants, using for example EMS mutagenesis; alternatively, the programme may start with a collection of allelic variants of so called “natural” origin caused unintentionally. Identification of allelic variants then takes place, for example, by PCR. This is followed by a step for selection of superior allelic variants of the sequence in question and which give increased yield. Selection is typically carried out by monitoring growth performance of plants containing different allelic variants of the sequence in question, for example, different allelic variants of any one of the nucleic acids given in Table 1.

Growth performance may be monitored in a greenhouse or in the field. Further optional steps include crossing plants, in which the superior allelic variant was identified, with another plant. This could be used, for example, to make a combination of interesting phenotypic features.

A two-WRKY domain nucleic acid or variant thereof may also be used as probes for genetically and physically mapping the genes that they are a part of, and as markers for traits linked to those genes. Such information may be useful in plant breeding in order to develop lines with desired phenotypes. Such use of two-WRKY domain nucleic acids or variants thereof requires only a nucleic acid sequence of at least 15, 16, 17, 18, 19 or 20 nucleotides in length. The two-WRKY domain nucleic acids or variants thereof may be used as restriction fragment length polymorphism (RFLP) markers. Southern blots (Sambrook J, Fritsch E F and Maniatis T (1989) Molecular Cloning, A Laboratory Manual) of restriction-digested plant genomic DNA may be probed with the two-WRKY domain nucleic acids or variants thereof. The resulting banding motifs may then be subject to genetic analyses using computer programs such as MapMaker (Lander et al., (1987) Genomics 1: 174-181) in order to construct a genetic map. In addition, the nucleic acids may be used to probe Southern blots containing restriction endonuclease-treated genomic DNAs of a set of individuals representing parent and progeny of a defined genetic cross. Segregation of the DNA polymorphisms is noted and used to calculate the position of the two-WRKY domain nucleic acid or variant thereof in the genetic map previously obtained using this population (Botstein et al. (1980) Am. J. Hum. Genet. 32:314-331).

The production and use of plant gene-derived probes for use in genetic mapping is described in Bematzky and Tanksley (1986) Plant Mol. Biol. Reporter 4: 37-41. Numerous publications describe genetic mapping of specific cDNA clones using the methodology outlined above or variations thereof. For example, F2 intercross populations, backcross populations, randomly mated populations, near isogenic lines, and other sets of individuals may be used for mapping. Such methodologies are well known to those skilled in the art.

The nucleic acid probes may also be used for physical mapping (i.e., placement of sequences on physical maps; see Hoheisel et al. In: Non-mammalian Genomic Analysis: A Practical Guide, Academic press 1996, pp. 319-346, and references cited therein).

In another embodiment, the nucleic acid probes may be used in direct fluorescence in situ hybridization (FISH) mapping (Trask (1991) Trends Genet. 7:149-154). Although current methods of FISH mapping favor use of large clones (several kb to several hundred kb; see Laan et al. (1995) Genome Res. 5:13-20), improvements in sensitivity may allow performance of FISH mapping using shorter probes.

A variety of nucleic acid amplification-based methods for genetic and physical mapping may be carried out using the nucleic acids. Examples include allele-specific amplification [Kazazian (1989) J. Lab. Clin. Med 11:95-96], polymorphism of PCR-amplified fragments [CAPS; Sheffield et al. (1993) Genomics 16:325-332], allele-specific ligation [Landegren et al. (1988) Science 241:1077-1080], nucleotide extension reactions [Sokolov (1990) Nucleic Acid Res. 18:3671], Radiation Hybrid Mapping [Walter et al. (1997) Nat. Genet. 7:22-28] and Happy Mapping [Dear and Cook (1989) Nucleic Acid Res. 17:6795-6807]. For these methods, the sequence of a nucleic acid is used to design and produce primer pairs for use in the amplification reaction or in primer extension reactions. The design of such primers is well known to those skilled in the art. In methods employing PCR-based genetic mapping, it may be necessary to identify DNA sequence differences between the parents of the mapping cross in the region corresponding to the instant nucleic acid sequence. This, however, is generally not necessary for mapping methods.

The methods according to the present invention result in plants having increased yield, as described hereinbefore. These advantageous growth characteristics may also be combined with other economically advantageous traits, such as further yield-enhancing traits, tolerance to various stresses, traits modifying various architectural features and/or biochemical and/or physiological features.

### DESCRIPTION OF FIGURES

The present invention will now be described with reference to the following figures in which:

FIG. 1 shows the typical domain structure of a polypeptide having two WRKY domains. The Pro-Ser rich domain is located at the amino-terminal end of the protein; the LXSP motif (L for Leu, S for Ser, P for Pro and X for any amino acid) is contained in this region and is indicated. The two-WRKY domains are boxed in black. Between the two-WRKY domains are the acidic (AC) stretch and the putative nuclear localization signal (NLS). The motif of SEQ ID NO: 39 which represents the carboxy-terminal WRKY domain is also boxed.

FIG. 2 is the phylogenetic analysis of 58 members of the Arath_WRKY family (from Eulgem et al. (2000) Trends Plant Sci 5(5): 199-206). The black arrow indicates the cluster of polypeptides having two WRKY domains and a Pro-Ser rich domain at their amino-terminus.

FIG. 3 shows a multiple alignment of several polypeptides having two WRKY domains created using VNTI AlignX multiple alignment program, based on a modified ClustalW algorithm (InforMax, Bethesda, Md., http://www.informaxinc.com), with default settings for gap opening penalty of 10 and a gap extension of 0.05). Minor manual editing was also carried out where necessary to better position some conserved regions. The important domains from amino-terminus to carboxy-terminus are boxed across the plant polypeptides: the Pro-Ser rich domain and its LXSP motif (where L is Leu, S is Ser, P is Pro and X is any amino acid), the amino-terminal WRKY domain (and its heptapeptide) including its C₂H₂ zinc binding domain, the acidic stretch, the NLS, the motif of SEQ ID NO: 39, and the carboxy-terminal WRKY domain (and its heptapeptide) including its C₂H₂ zinc binding domain are either boxed or written in bold. The LXSP motif of SEQ ID NO: 2 is from amino acid to amino acid, the acidic stretch from amino acid 304 to amino acid 309, the NLS from amino acid 311 to amino acid 314.

FIG. 4 shows a binary vector p0700 and p0709, for expression in Oryza sativa of an Oryza sativa polypeptide having two WRKY domains under the control of respectively a GOS2 promoter (internal reference PRO0129; represented as in SEQ ID NO: 42) and an oleosin promoter (internal reference PRO0218; represented as in SEQ ID NO: 43).

FIG. 5 details examples of sequences useful in performing the methods according to the present invention, from start codon to stop codon in case of (full length) polynucleotides sequences encoding polypeptides with two_WRKY domains.

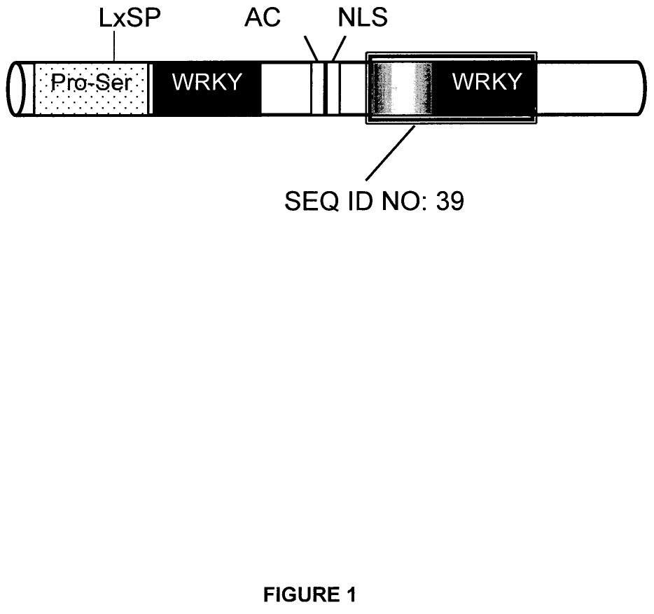

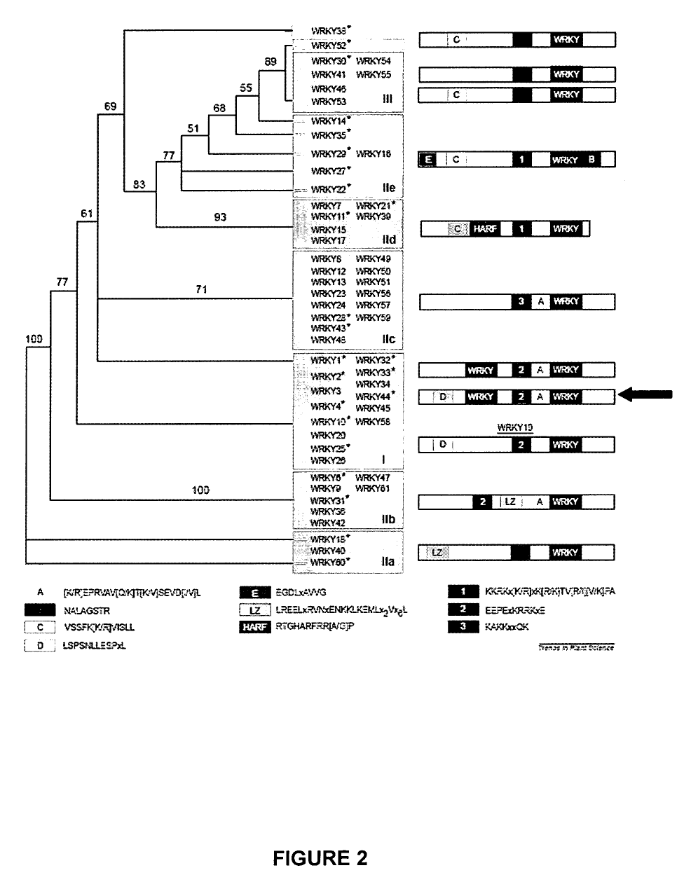

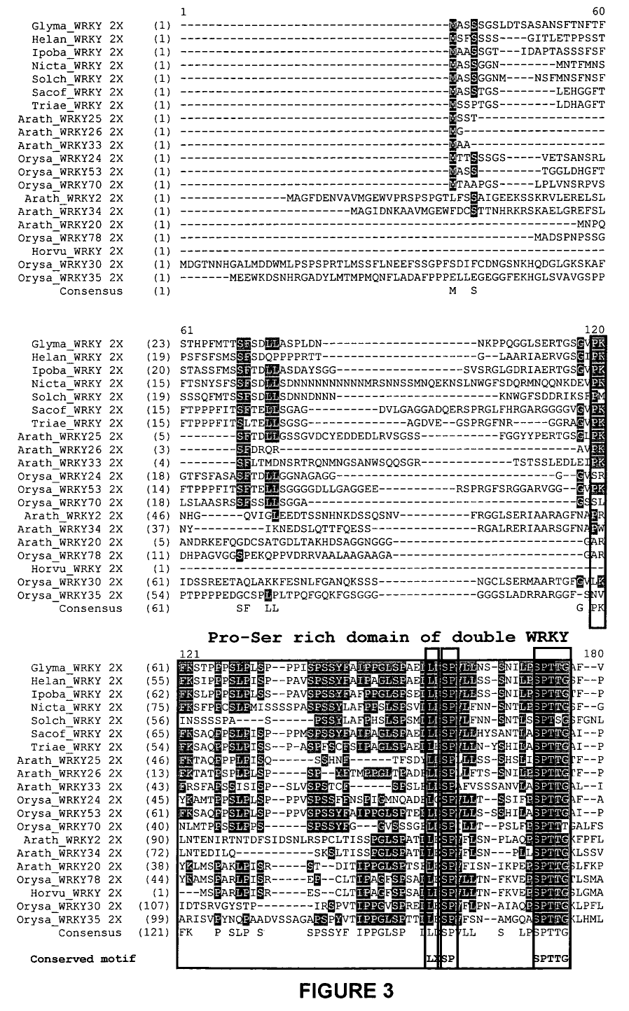

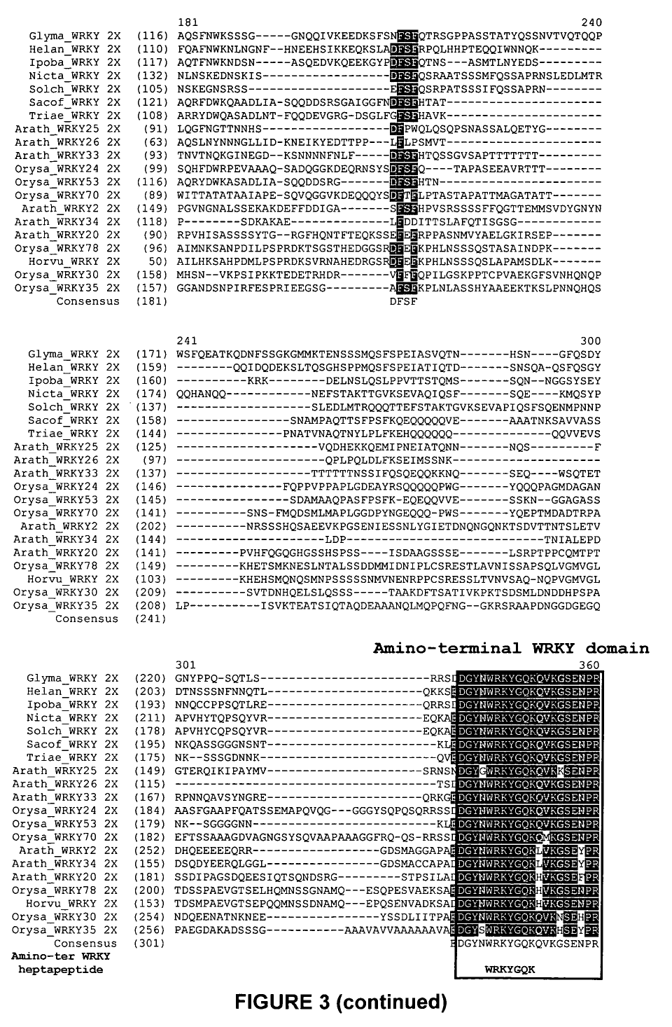

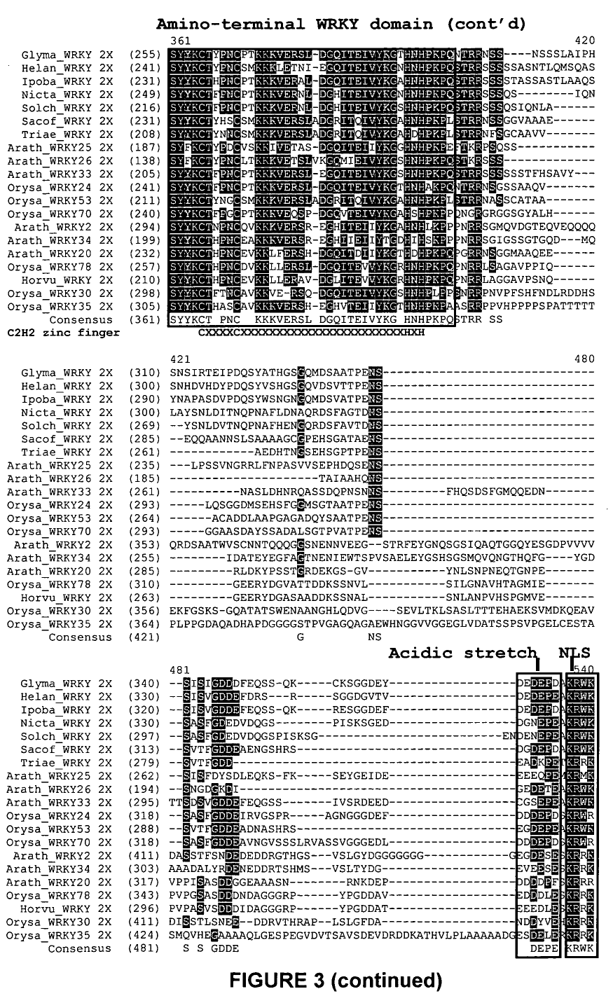

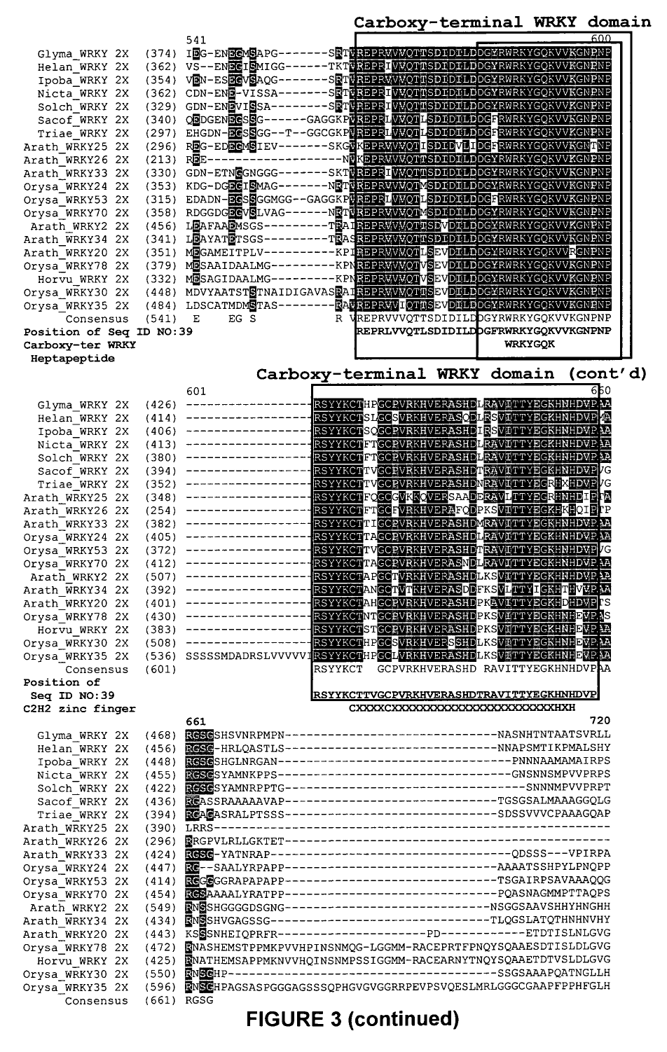

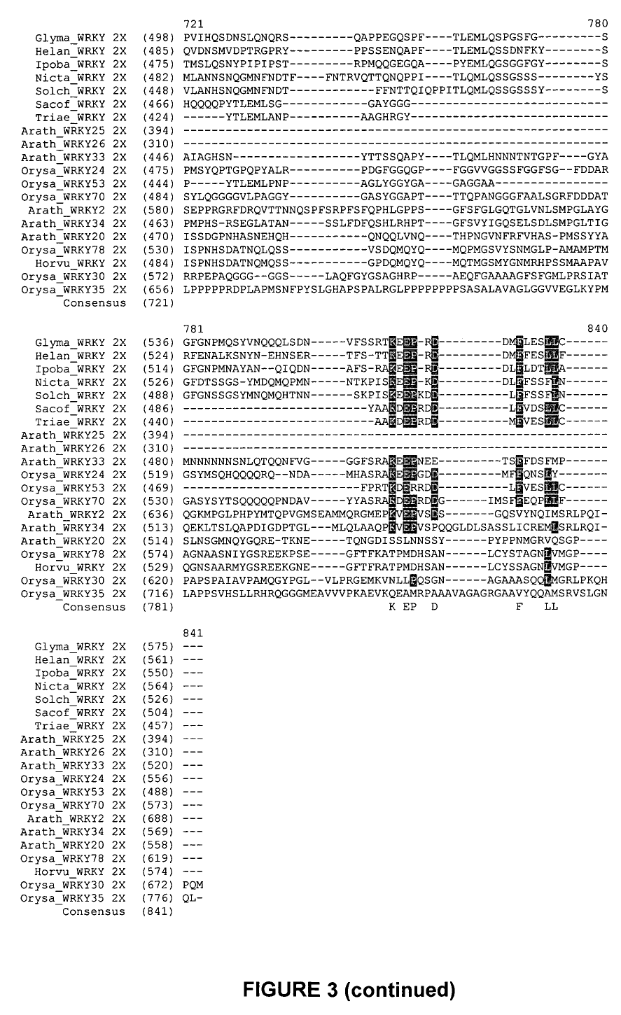

### EXAMPLES

The present invention will now be described with reference to the following examples, which are by way of illustration alone.

DNA manipulation: unless otherwise stated, recombinant DNA techniques are performed according to standard protocols described in (Sambrook (2001) Molecular Cloning: a laboratory manual, 3rd Edition Cold Spring Harbor Laboratory Press, CSH, New York) or in Volumes 1 and 2 of Ausubel et al. (1994), Current Protocols in Molecular Biology, Current Protocols. Standard materials and methods for plant molecular work are described in Plant Molecular Biology Labfax (1993) by R. D. D. Croy, published by BIOS Scientific Publications Ltd (UK) and Blackwell Scientific Publications (UK).

### Example 1

### Cloning of the Oryza sativa Two-WRKY Domain Gene

The Oryza sativa two-WRKY domain gene of SEQ ID NO: 1 was amplified by PCR using as template an Oryza sativa seedling cDNA library (Invitrogen, Paisley, UK). After reverse transcription of RNA extracted from seedlings, the cDNAs were cloned into pCMV Sport 6.0. Average insert size of the bank was 1.66 kb and the original number of clones was of the order of 2.67×10⁷ cfu. Original titer was determined to be 3.34×10⁶ cfu/ml, and after a first amplification of 10¹⁰ cfu/ml. After plasmid extraction, 200 ng of template was used in a 50 μl PCR mix. Primers prm05769 (SEQ ID NO: 40; sense, start codon in bold, AttB1 site in italic: 5′-GGGGACAA GTTTGTACAAAAAAGCAGGCTTAAACAATGGCGTCCTCGACG 3′) and prm05770 (SEQ ID NO: 41; reverse, complementary, AttB2 site in italic: 5′ GGGGACCACTTTGTACAAGAAAGCTGGGTGGCTCGACTAGCAGAGGA 3′), which include the AttB sites for Gateway recombination, were used for PCR amplification. PCR was performed using Hifi Taq DNA polymerase in standard conditions. A PCR fragment of 1535 bp (including attB sites) was amplified and purified also using standard methods. The first step of the Gateway procedure, the BP reaction, was then performed, during which the PCR fragment recombines in vivo with the pDONR201 plasmid to produce, according to the Gateway terminology, an “entry clone”, p06983. Plasmid pDONR201 was purchased from Invitrogen, as part of the Gateway® technology.

### Example 2

### Vector Construction

The entry clone p06983 was subsequently used in an LR reaction with p00640, a destination vector used for Oryza sativa transformation. This vector contains as functional elements within the T-DNA borders: a plant selectable marker; a screenable marker expression cassette; and a Gateway cassette intended for LR in vivo recombination with the sequence of interest already cloned in the entry clone. A rice GOS2 promoter (SEQ ID NO: 42) for constitutive expression (PRO0129) was located upstream of this Gateway cassette.

The entry clone p06983 was used in a second LR reaction with p00831, another destination vector used for Oryza sativa transformation. A rice 18 kDa oleosin promoter (SEQ ID NO: 43) for embryo and/or aleurone specific expression (PRO0218) was located upstream of the Gateway cassette.

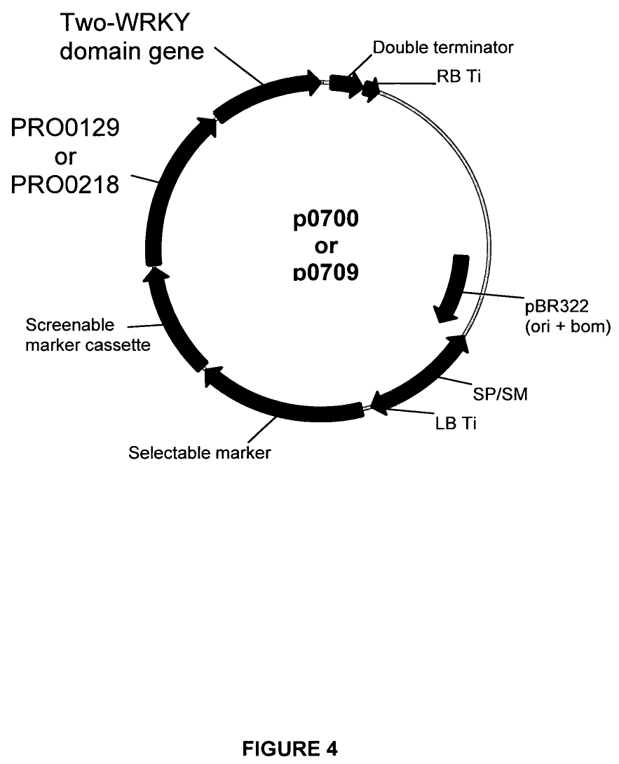

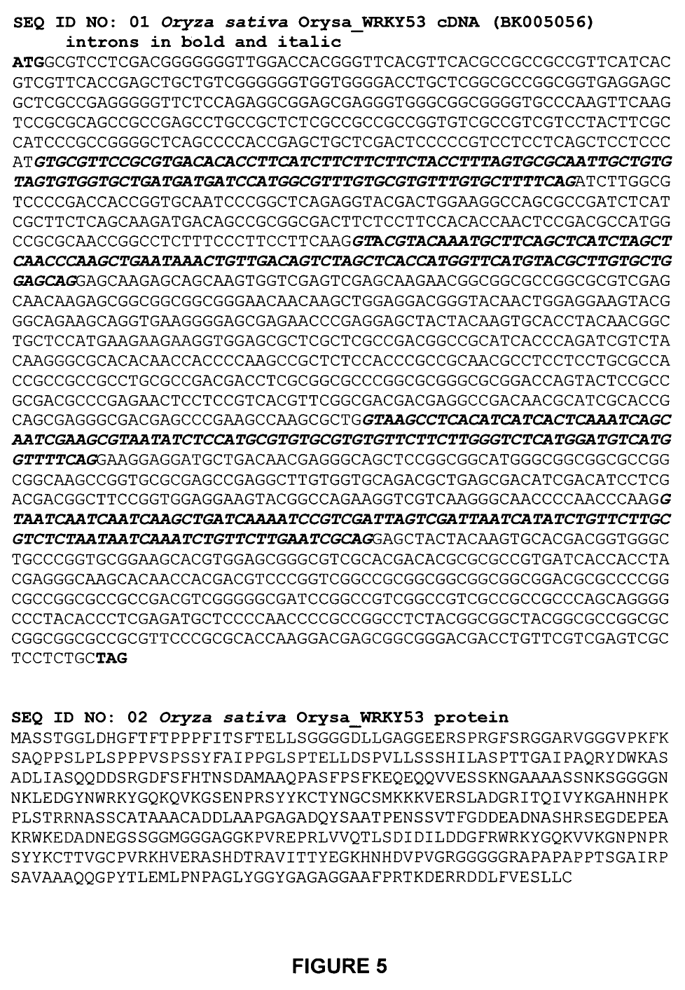

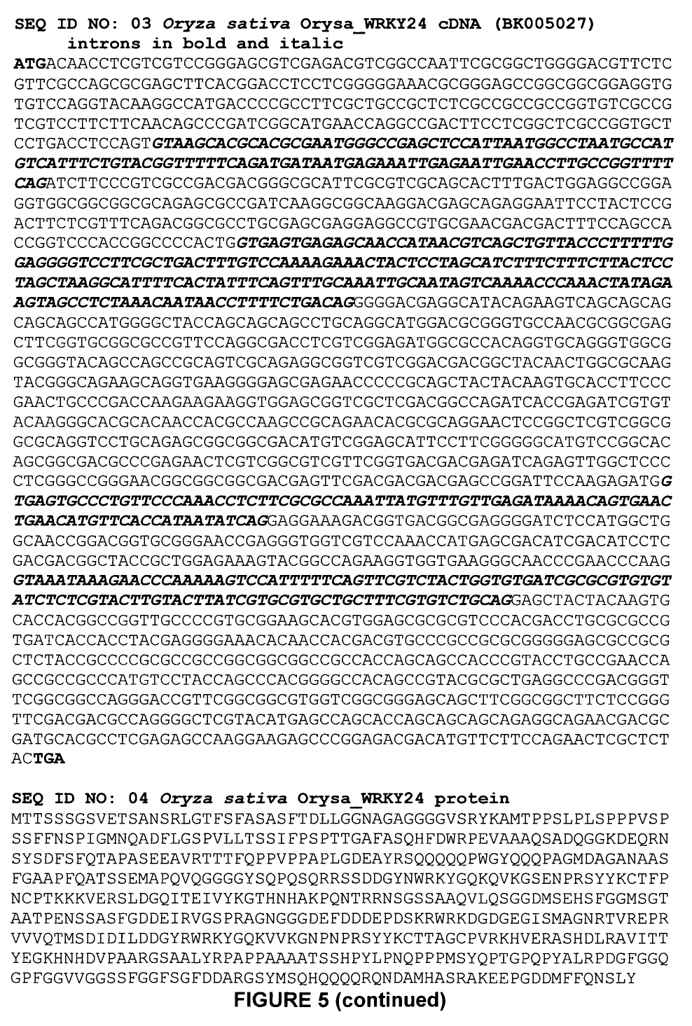

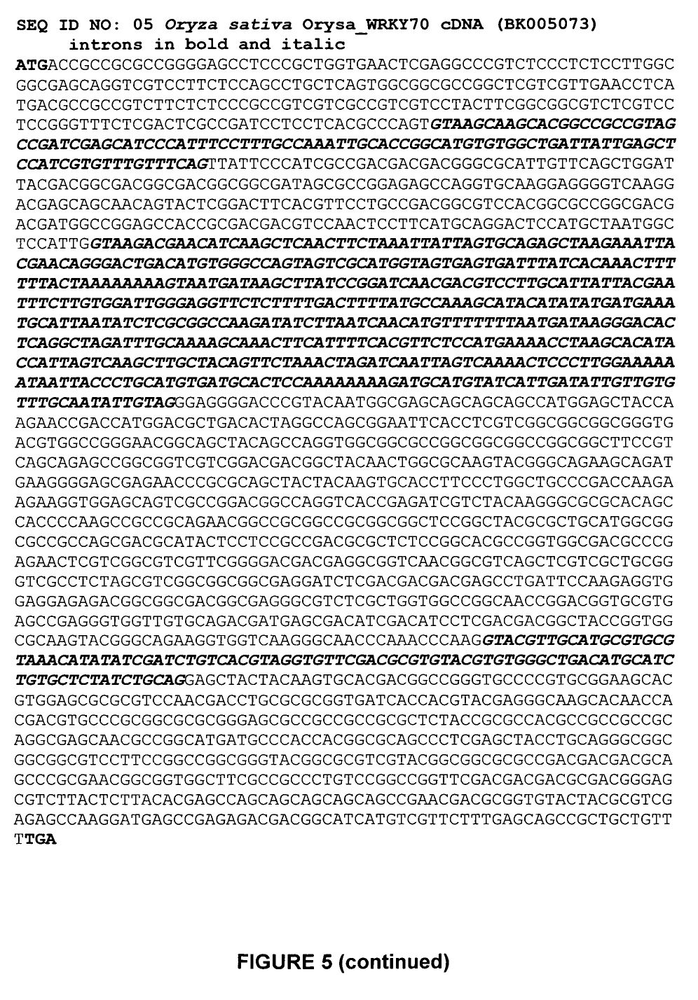

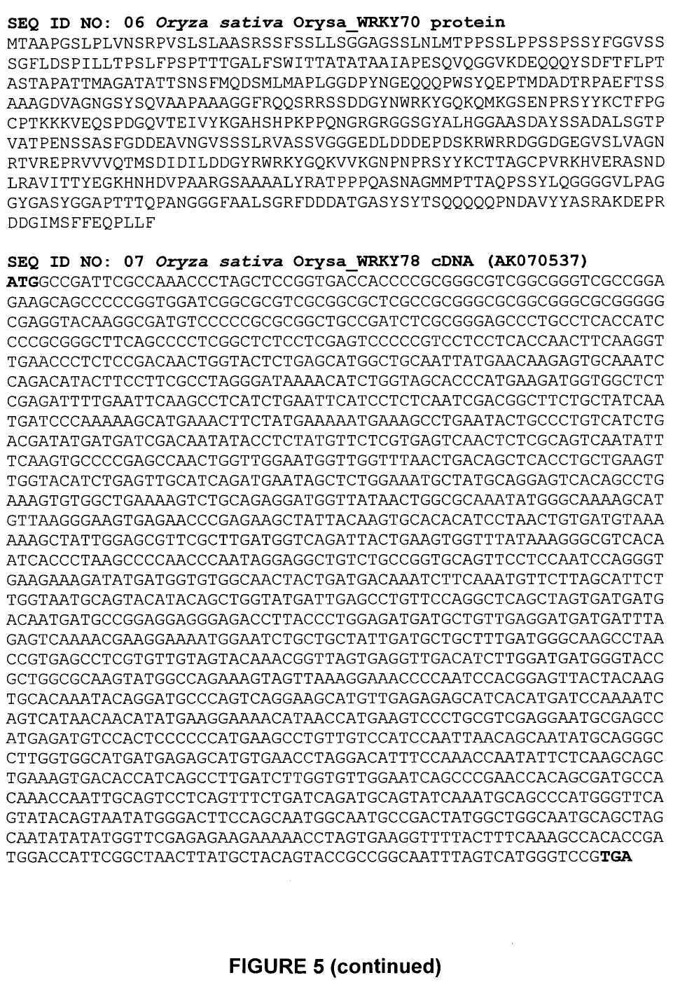

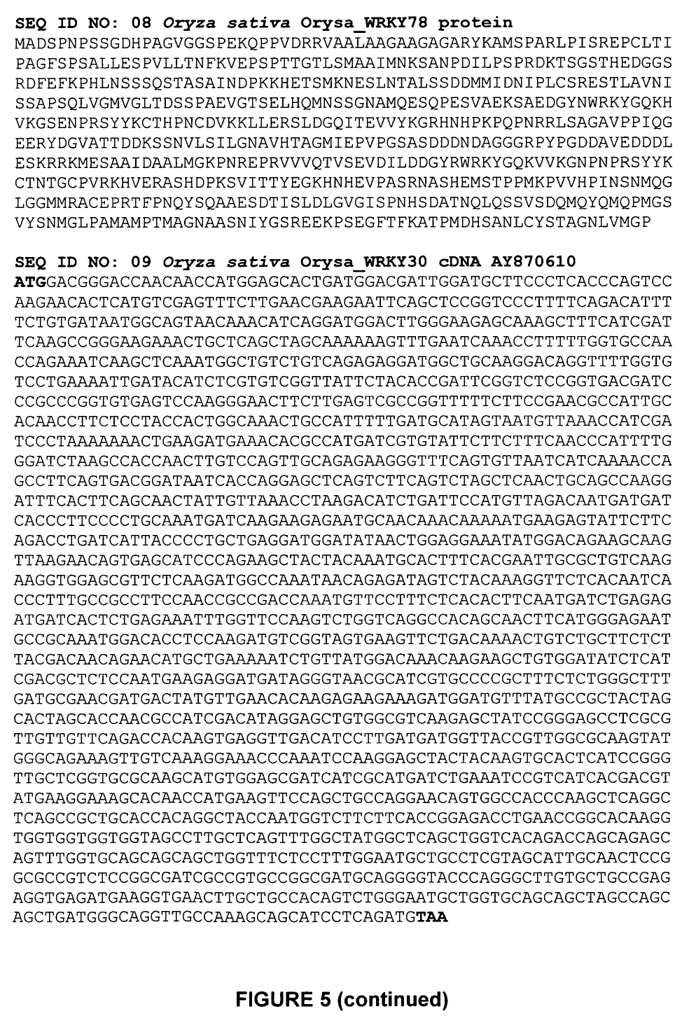

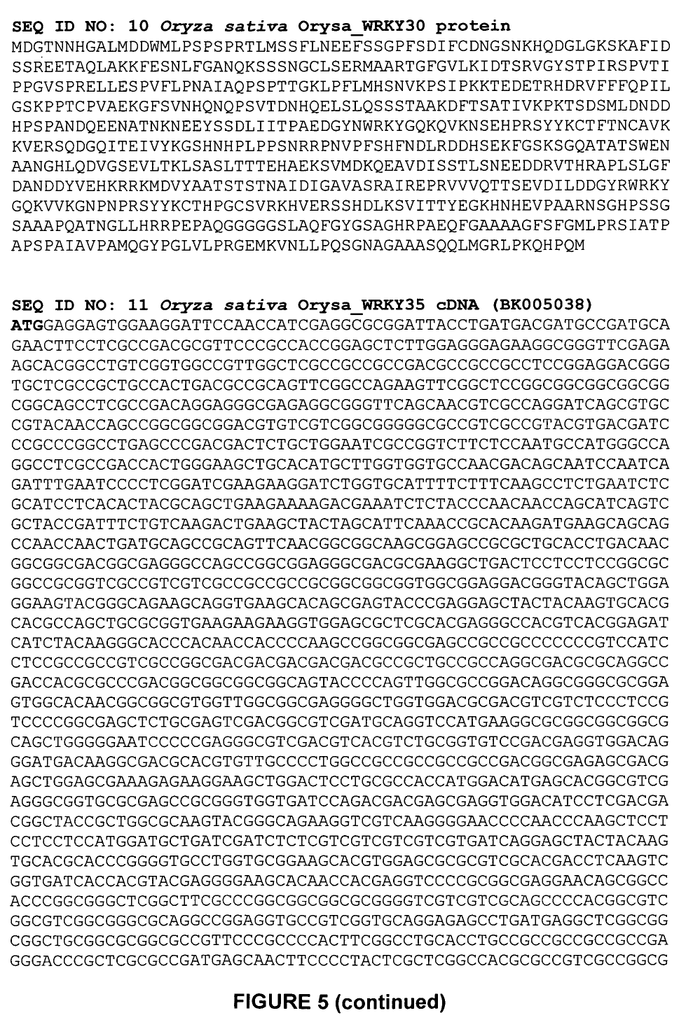

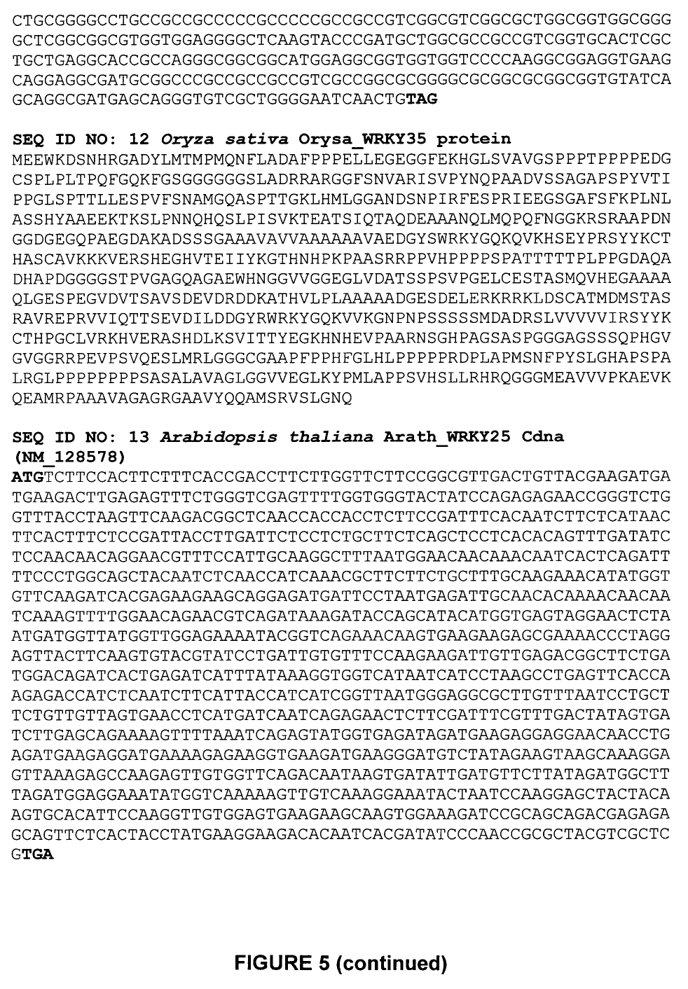

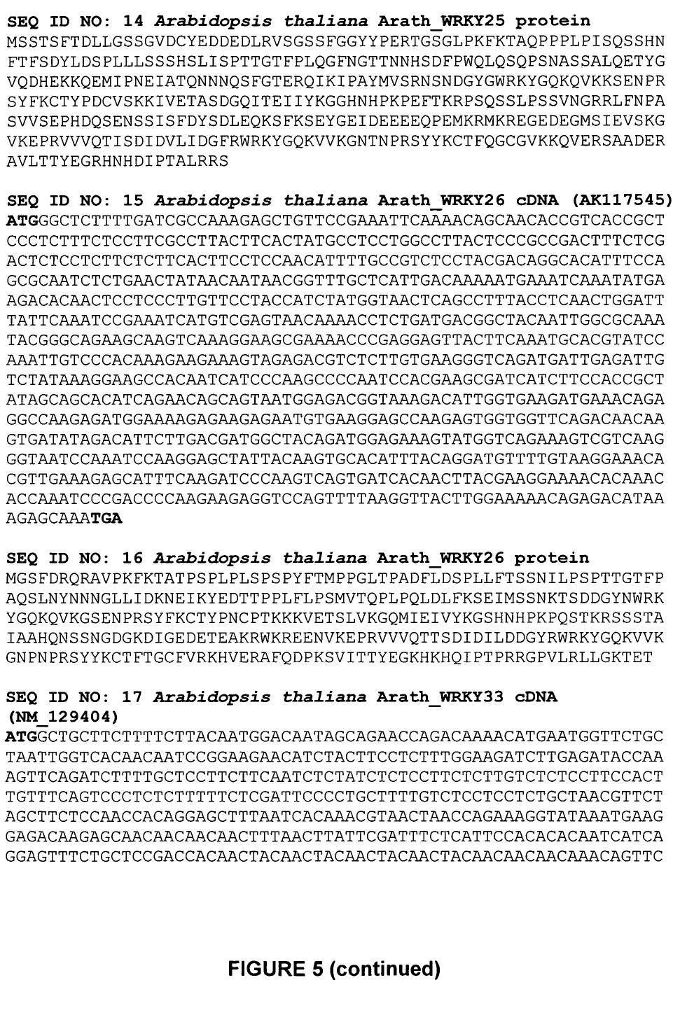

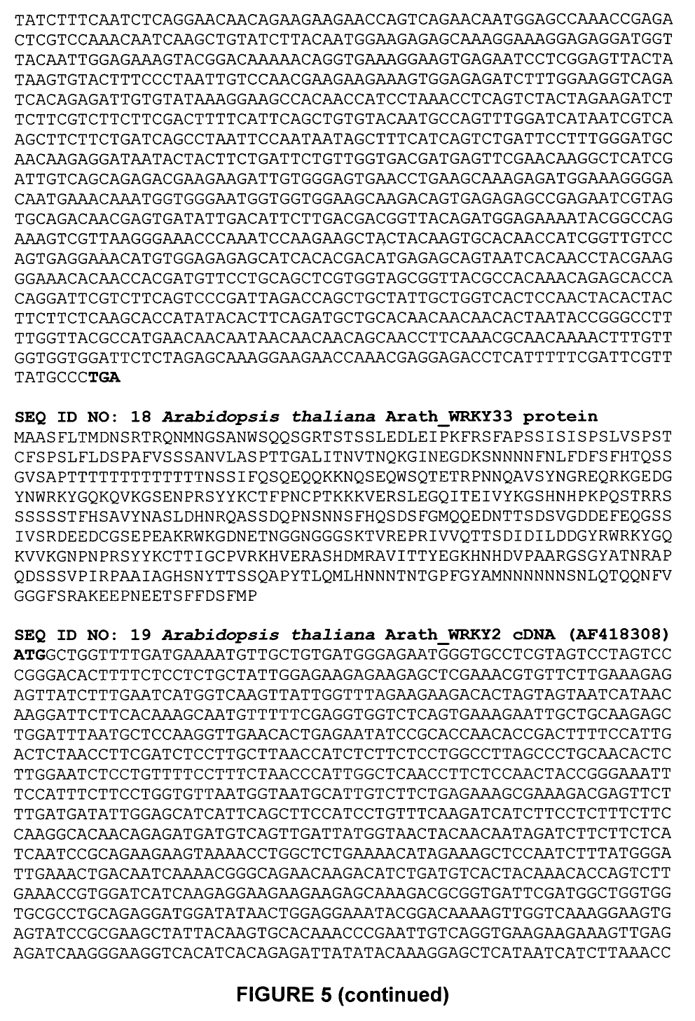

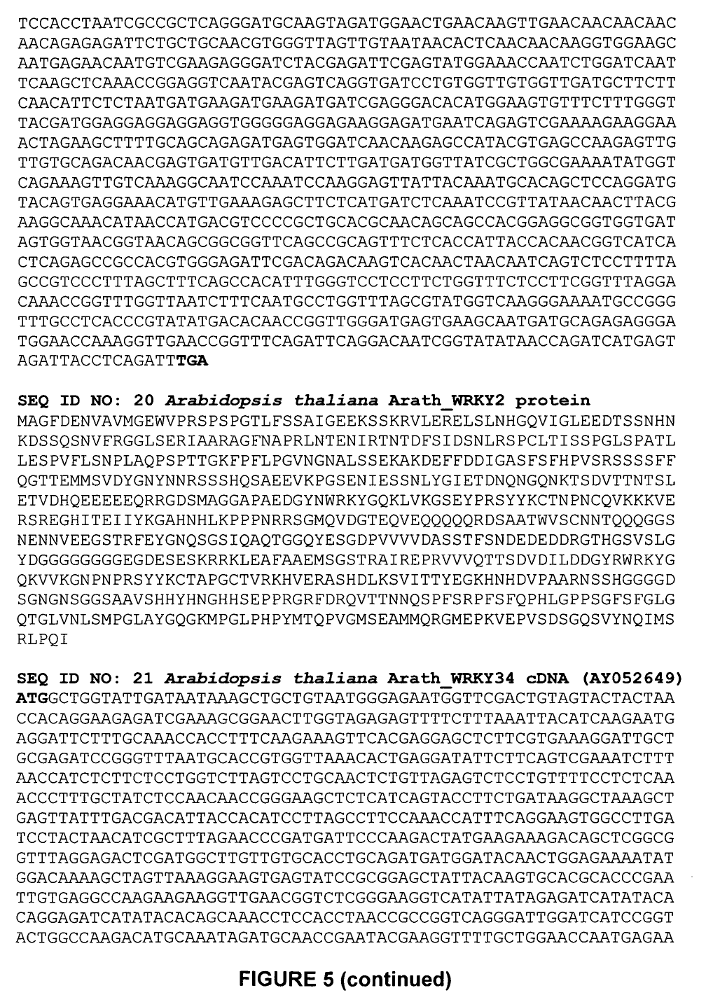

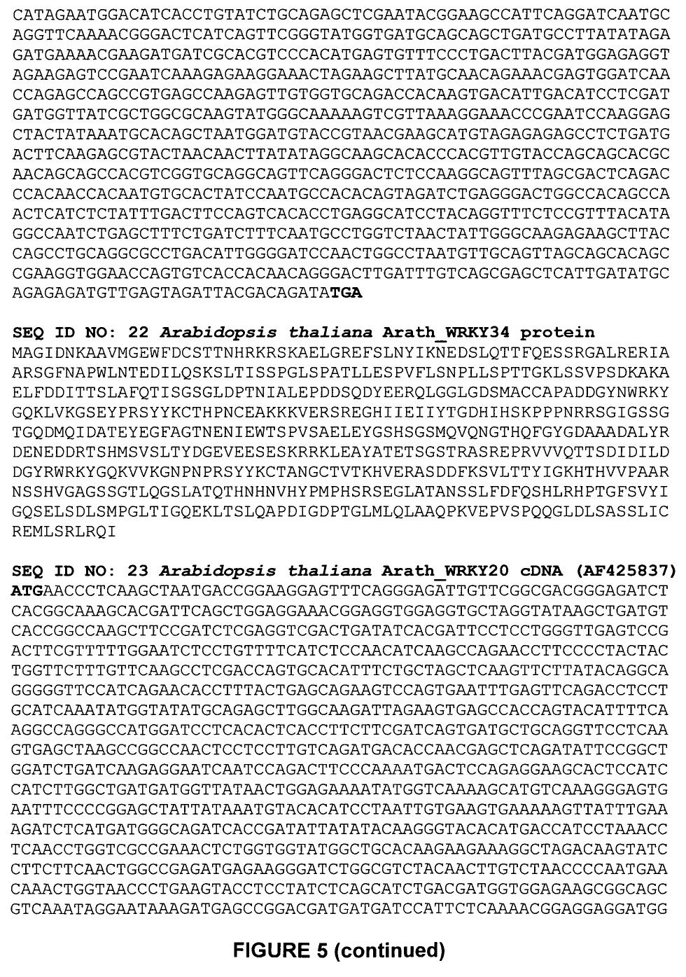

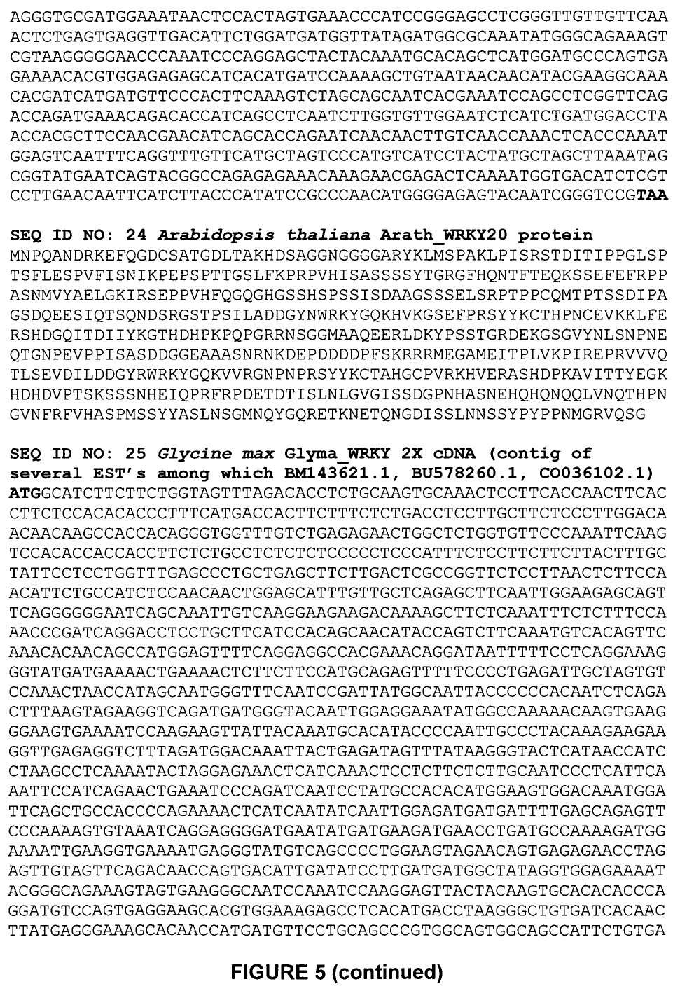

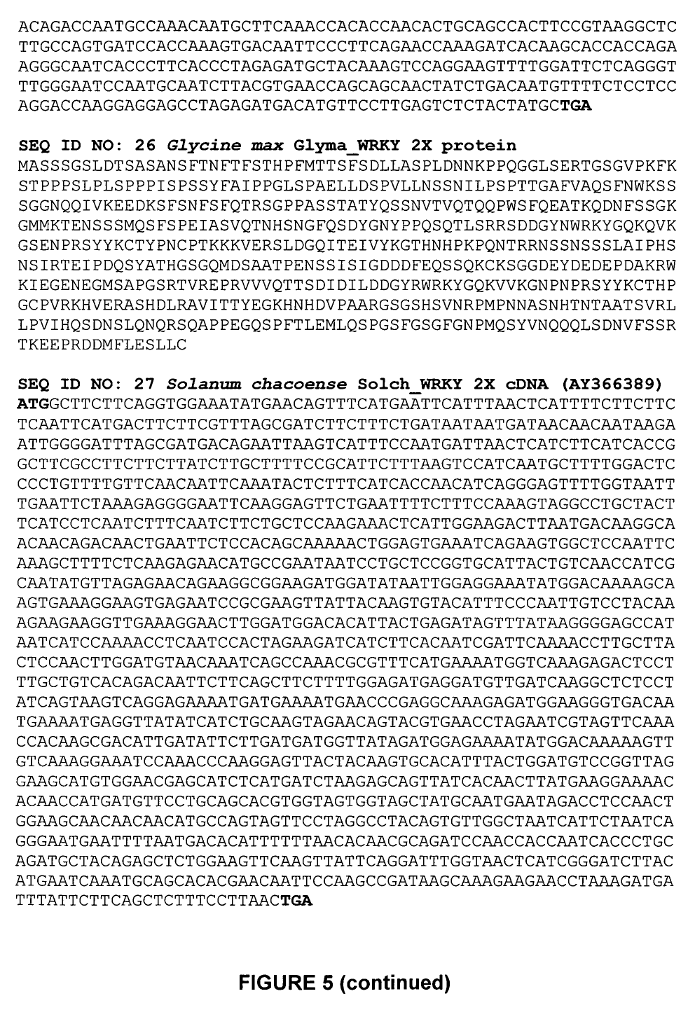

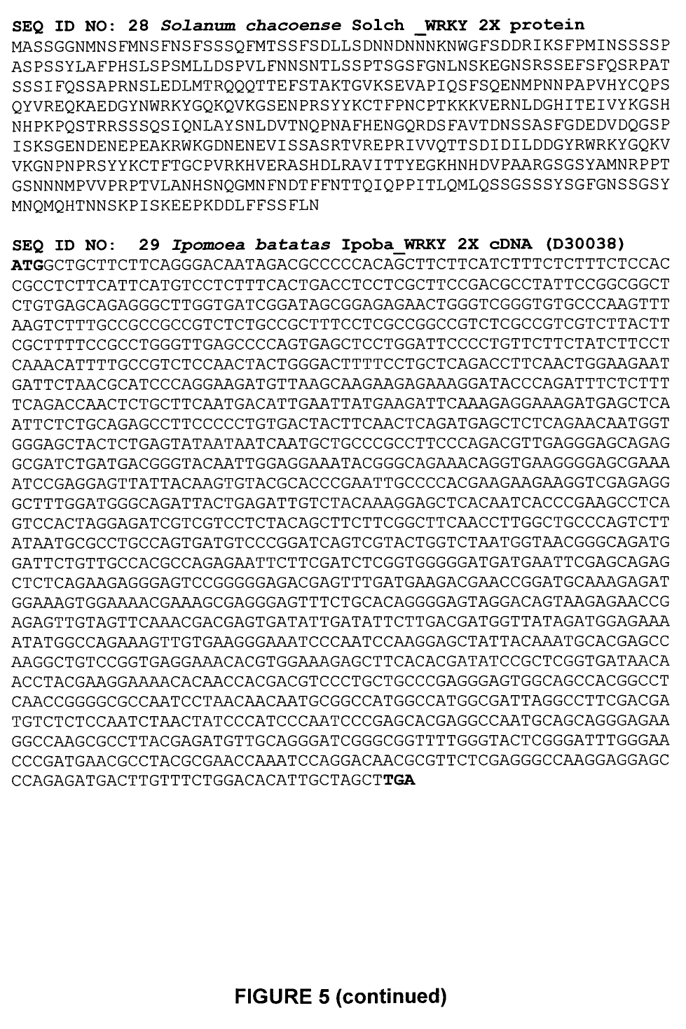

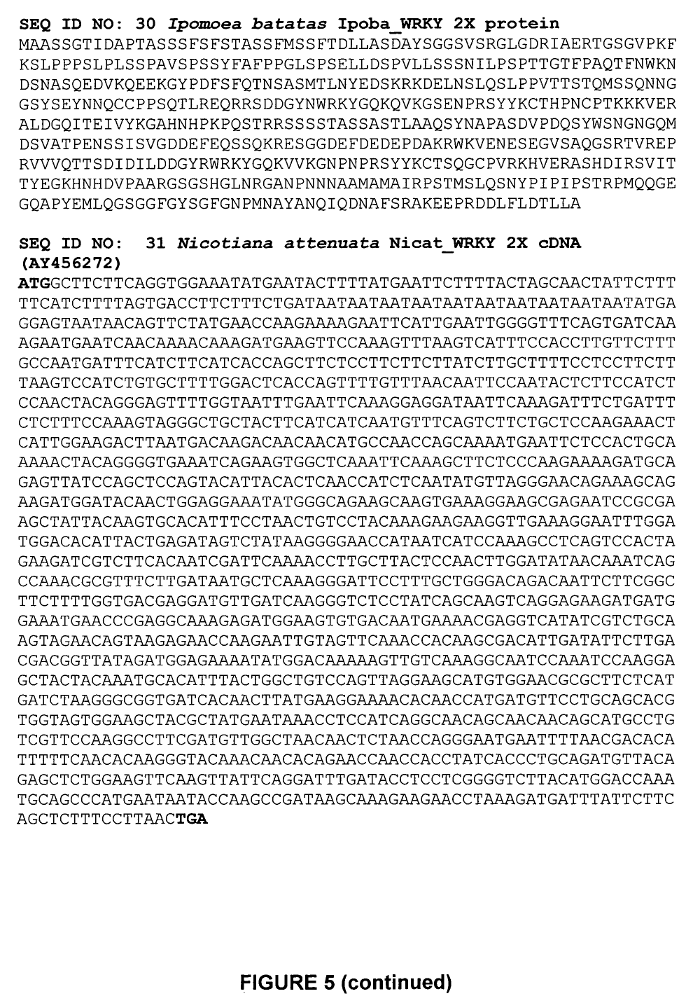

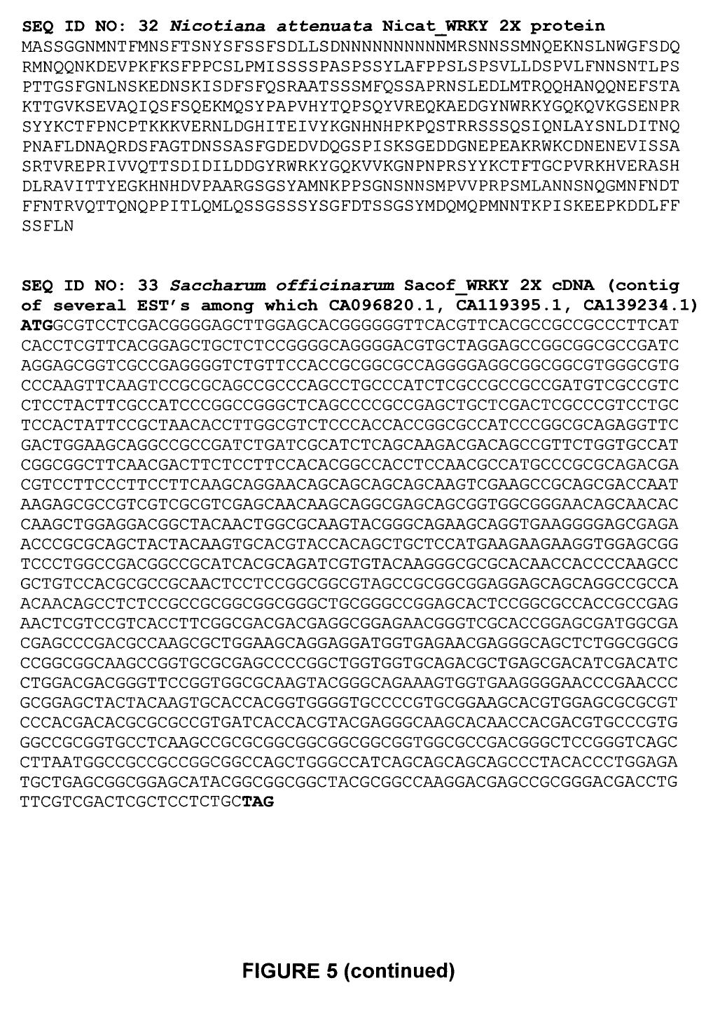

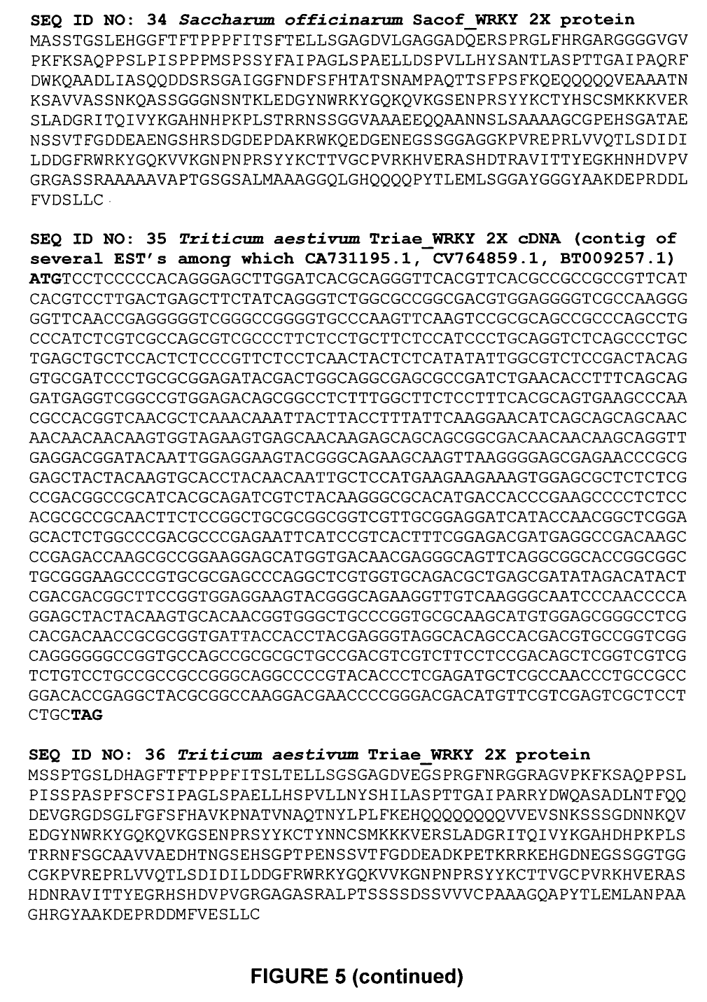

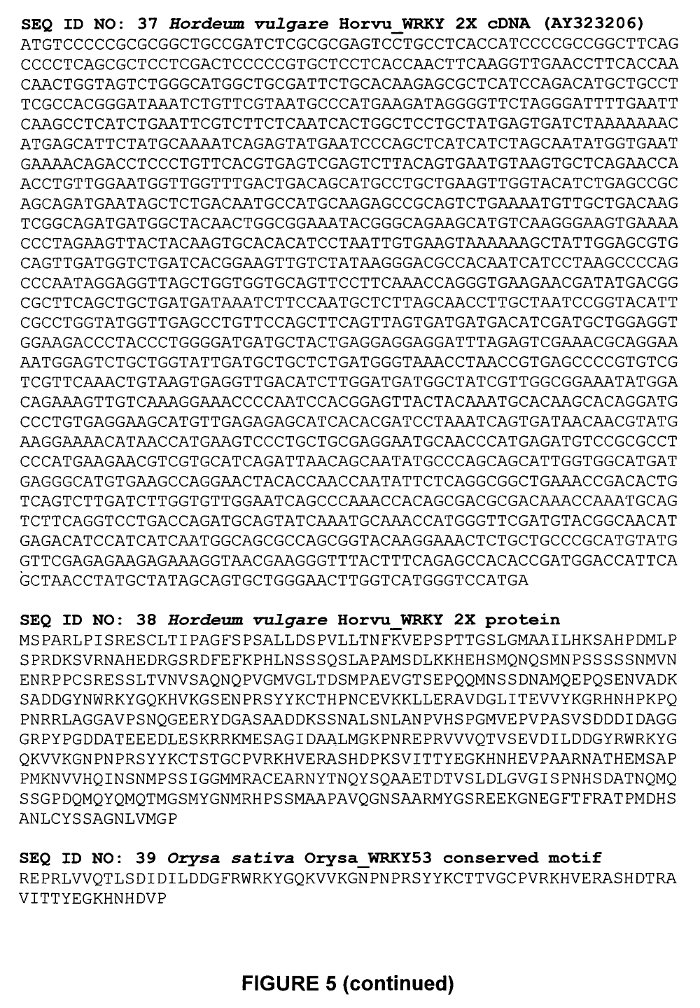

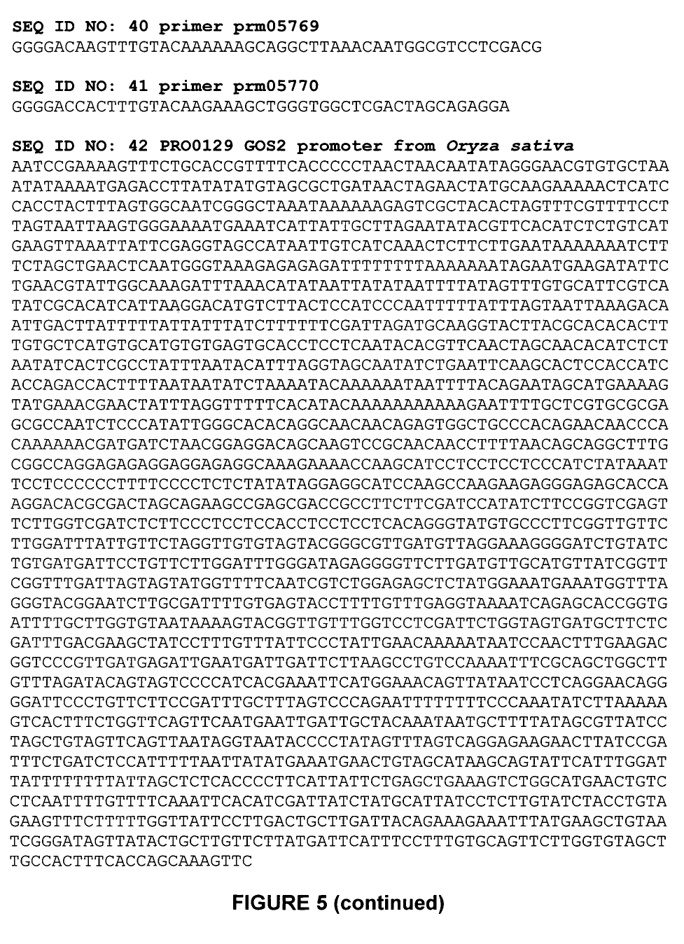

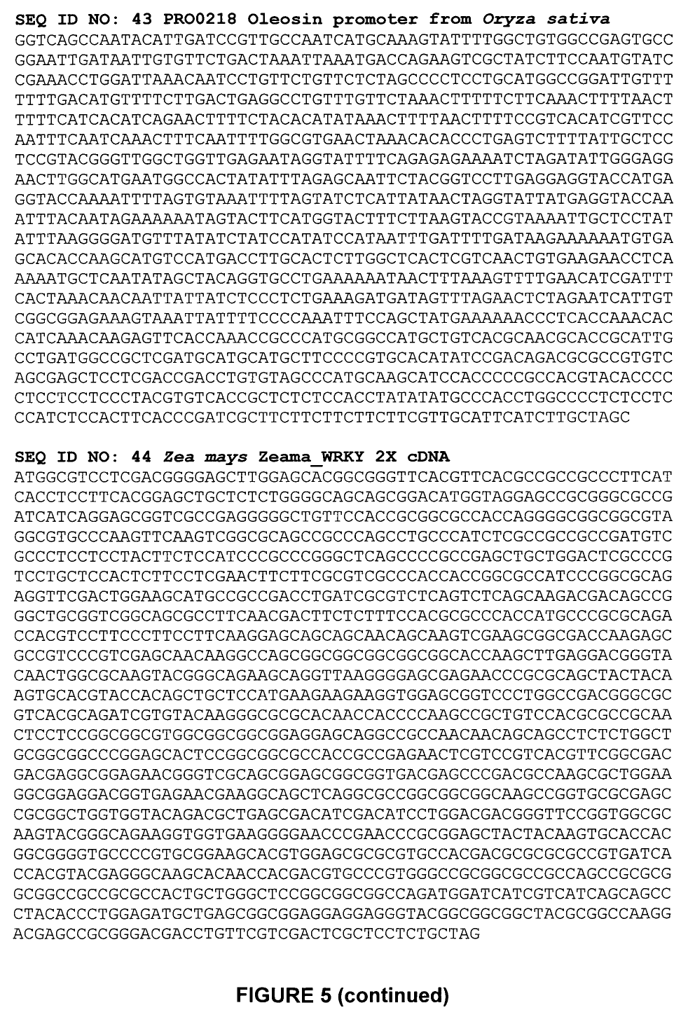

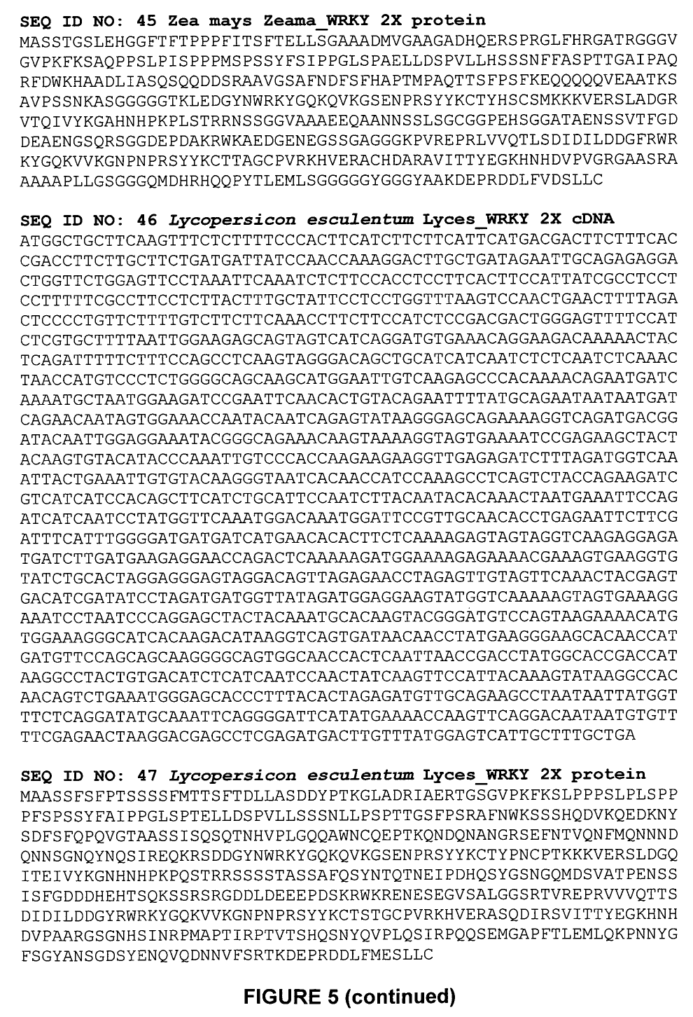

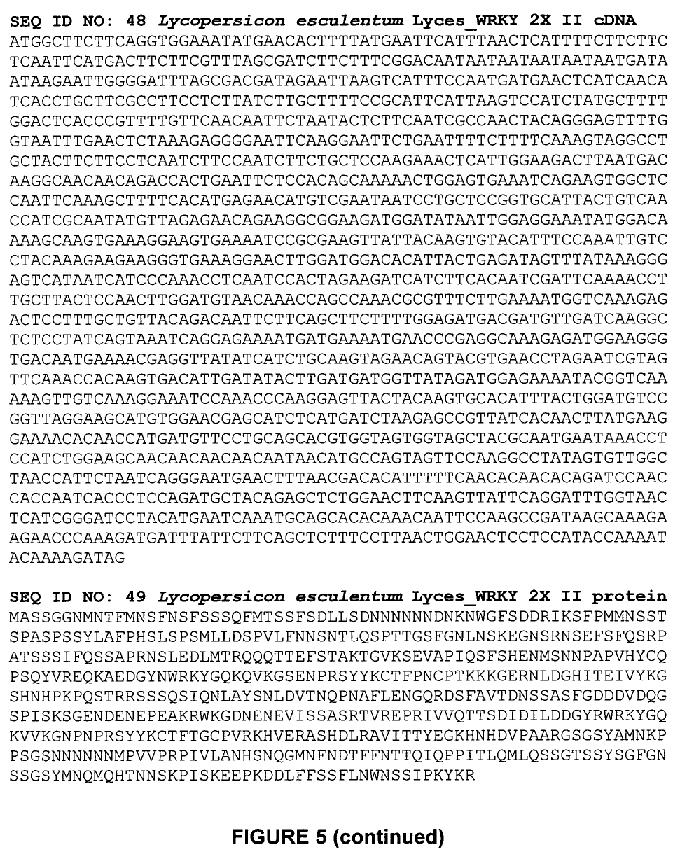

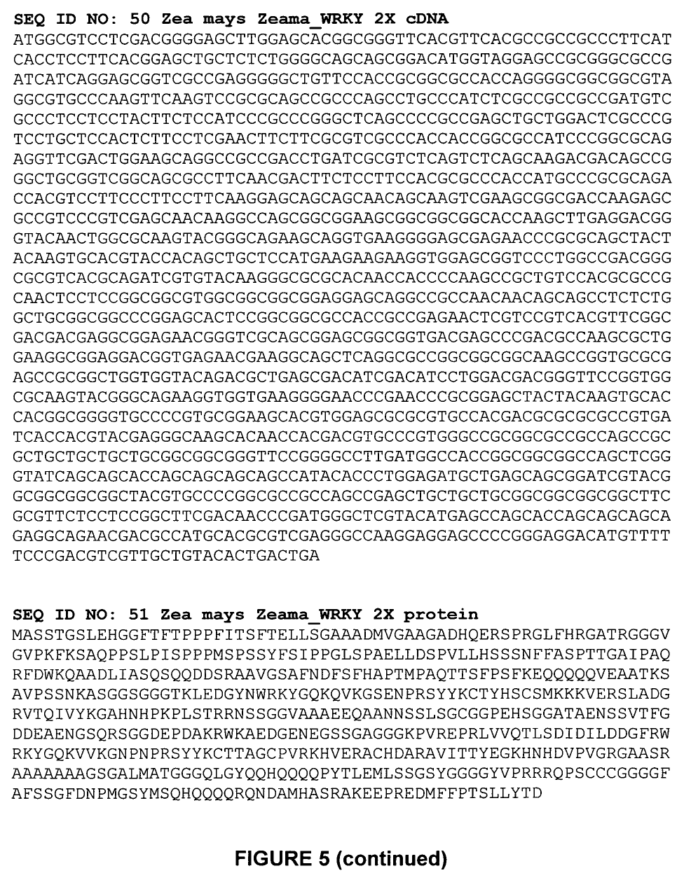

After the LR recombination step, the resulting expression vectors p0700 and p0709 (FIG. 4) were separately transformed into Agrobacterium strain LBA4044 and subsequently separately to Oryza sativa plants. Transformed rice plants were allowed to grow and were then examined for the parameters described in Example 3.

### Example 3

### Evaluation and Results of the Oryza sativa Two-WRKY Domain Transgenic Plants

Approximately 15 to 20 independent T0 rice transformants were generated. The primary transformants were transferred from a tissue culture chamber to a greenhouse for growing and harvest of T1 seed. Four to five events, of which the T1 progeny segregated 3:1 for presence/absence of the transgene, were retained. For each of these events, approximately 10 T1 seedlings containing the transgene (hetero- and homo-zygotes) and approximately 10 T1 seedlings lacking the transgene (nullizygotes) were selected by monitoring visual marker expression. The transgenic plants and the corresponding nullizygotes were grown side-by-side at random positions. From the stage of sowing until the stage of maturity the plants were passed several times through a digital imaging cabinet. At each time point, digital images (2048×1536 pixels, 16 million colours) were taken of each plant from at least 6 different angles.

Four T1 events were further evaluated in the T2 generation following the same evaluation procedure as for the T1 generation but with more individuals per event.

### Statistical Analysis: F-Test

A two factor ANOVA (analysis of variants) was used as a statistical model for the overall evaluation of plant phenotypic characteristics. An F-test was carried out on all the parameters measured of all the plants of all the events transformed with the gene of the present invention. The F-test was carried out to check for an effect of the gene over all the transformation events and for an overall effect of the gene, also known as a global gene effect. The threshold for significance for a true global gene effect was set at a 5% probability level for the F-test. A significant F-test value points to a gene effect, meaning that it is not only the presence or position of the gene that is causing the differences in phenotype.

### Seed-Related Parameter Measurements

The mature primary panicles were harvested, bagged, barcode-labeled and then dried for three days in an oven at 37° C. The panicles were then threshed and all the seeds were collected and counted, giving the total number of seeds. The total number of seeds divided by the number of primary panicles provided for an estimation of the number of florets per panicle. The filled husks were then separated from the empty ones using an air-blowing device. The empty husks were discarded and the remaining fraction counted again. The filled husks were weighed on an analytical balance. The number of filled seeds was determined by counting the number of filled husks that remained after the separation step. The total seed yield was measured by weighing all filled husks harvested from a plant. Thousand Kernel Weight (TKW) was extrapolated from the number of filled seeds counted and their total weight. Harvest index was derived from the ratio of total seed yield and the aboveground area (mm²) multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets). Plant aboveground was determined by counting the total number of pixels from the pictures of the aboveground plant parts discriminated from the background. This value was averaged for the pictures taken on the same time point from the different angles and converted to a physical surface value expressed in square mm by calibration. Experiments show that the aboveground plant area measured this way correlates with the biomass of plant.

3.1 Evaluation and Results of the Oryza sativa Transgenic Plants with a Constitutive Promoter Upstream of a Nucleic Acid Encoding a Polypeptide with Two-WRKY Domains

The TKW measurement results for the Oryza sativa two-WRKY domain transgenic plants are shown in Table 6. The percentage difference between the transgenics and the corresponding nullizygotes is also shown. The number of events with a significant increase in TKW is indicated, as well as the P values from the F test for the T1 and the T2 generations.

The TKW was significantly increased in the T1 and T2 generations for the Oryza sativa two-WRKY domain transgenic plants compared their null counterparts (Table 6).

TABLE 6
Results of TKW measurements in the T1 and T2 generation of the
Oryza sativa two-WRKY domain transgenic plants compared
to their null counterparts.

| | Number of | | Number of events | P |
|---|---|---|---|---|
| | events showing | % | showing a significant | value of |
| | an increase | Difference | increase | F test |
| T1 | 5 out of 5 | 3 | 2 out of 5 | <0.001 |
| generation | | | | |
| T2 | 4 out of 4 | 6 | 4 out of 4 | <0.001 |
| generation | | | | |

Individual seed parameters (width, length and area) were measured on the seeds from the T2 plants, using a custom-made device consisting of two main components, a weighing and imaging device, coupled to software for image analysis.

The average individual seed area, length and width measurement results of the T3 seeds (harvested from the T2 plants) for the Oryza sativa two-WRKY domain transgenic plants are shown in Table 7. The percentage difference between the transgenics and the corresponding nullizygotes is also shown. The number of events with a significant increase in a parameter is indicated, as well as the p values from the F test.

The average individual seed area, length and width of the T3 seeds of the Oryza sativa two-WRKY domain T2 transgenic plants were all significantly increased compared their null counterparts (Table 7).

TABLE 7
Individual seed area, length and width measurements of the
T3 seeds (harvested from the T2 plants) of the Oryza sativa
two-WRKY domain T2 transgenic plants compared to
their null counterparts.

| | Number of | | Number of events | P |
|---|---|---|---|---|
| | events showing | % | showing a | value of |
| | an increase | Difference | significant increase | F test |
| Average | 4 out of 4 | 3 | 4 out of 4 | <0.001 |
| seed area | | | | |
| Average | 4 out of 4 | 2 | 4 out of 4 | <0.001 |
| seed length | | | | |
| Average | 4 out of 4 | 1 | 2 out of 4 | <0.001 |
| seed width | | | | |

3.2 Evaluation and Results of the Oryza sativa Transgenic Plants with an Embryo and/or Aleurone Specific Promoter Upstream of a Nucleic Acid Encoding a Polypeptide with Two-WRKY Domains

The total number of seeds measurement results for the Oryza sativa two-WRKY domain transgenic plants are shown in Table 8. The percentage difference between the transgenics and the corresponding nullizygotes is also shown. The number of events with a significant increase in total number of seeds is indicated, as well as the P values from the F test for the T1 and the T2 generations.

The total number of seeds was significantly increased in the T1 and T2 generations for the Oryza sativa two-WRKY domain transgenic plants compared their null counterparts (Table 8).

TABLE 8
Results of total number of seeds measurements in the
T1 and T2 generation of the Oryza sativa two-WRKY
domain transgenic plants compared to their null
counterparts.

| | Number of | | Number of events | P |
|---|---|---|---|---|
| | events showing | % | showing a significant | value of |
| | an increase | Difference | increase | F test |
| T1 | 3 out of 4 | 11 | 2 out of 4 | 0.0037 |
| generation | | | | |
| T2 | 4 out of 4 | 12 | 2 out of 4 | 0.0029 |
| generation | | | | |

The total number of flowers per panicle measurement results for the Oryza sativa two-WRKY domain transgenic plants are shown in Table 9. The percentage difference between the transgenics and the corresponding nullizygotes is also shown. The number of events with a significant increase in total number of seeds is indicated, as well as the P values from the F test for the T1 and the T2 generations.

The total number of flowers per panicle was significantly increased in the T1 and T2 generations for the Oryza sativa two-WRKY domain transgenic plants compared their null counterparts (Table 9).

TABLE 9
Results of total number of flowers per panicle measurements
in the T1 and T2 generation of the Oryza sativa two-WRKY
domain transgenic plants compared to their null counterparts.

| | Number of | | Number of events | P |
|---|---|---|---|---|
| | events showing | % | showing a significant | value of |
| | an increase | Difference | increase | F test |
| T1 | 2 out of 4 | 7 | 2 out of 4 | 0.0122 |
| generation | | | | |
| T2 | 4 out of 4 | 7 | 1 out of 4 | 0.0091 |
| generation | | | | |

### Example 4

### Determination of Global Similarity and Identity Between Polypeptides Having Two-WRKY Domains Useful in Performing the Methods of the Invention

Global percentages of similarity and identity between polypeptides having two-WRKY domains were determined using one of the methods available in the art, the MatGAT (Matrix Global Alignment Tool) software (BMC Bioinformatics. 2003 4:29. MatGAT: an application that generates similarity/identity matrices using protein or DNA sequences. Campanella J J, Bitincka L, Smalley J; software hosted by Ledion Bitincka). MatGAT software generates similarity/identity matrices for DNA or protein sequences without needing pre-alignment of the data. The program performs a series of pair-wise alignments using the Myers and Miller global alignment algorithm (with a gap opening penalty of 12, and a gap extension penalty of 2), calculates similarity and identity using for example Blosum 62 (for polypeptides), and then places the results in a distance matrix. The sequence of SEQ ID NO: 2 is at line 17.

Results of the software analysis are shown in Table 10 for the global similarity and identity over the length of SEQ ID NO: 39 (conserved domain of 76 amino acids) of the polypeptides having two-WRKY domains. Percentage identity is given above the diagonal and percentage similarity is given below the diagonal. Percentage identity between the paralogues and orthologues having two WRKY domains ranges between 70 and 100%, reflecting the high sequence identity conservation between them within this conserved domain.

TABLE 10
Percentage identity and similarity of the conserved domain (as represented by
SEQ ID NO: 39) between orthologous and paralogous polypeptides having two
WRKY domains. Percentage identity is given above the diagonal and
percentage similarity is given below the diagonal.

| SEQ ID NO: 39 | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 |
|---|---|---|---|---|---|---|---|---|---|---|---|---|---|---|---|---|---|---|---|---|---|---|---|
| 1. SEQ ID NO: | | 90 | 72 | 86 | 90 | 88 | 93 | 91 | 91 | 92 | 91 | 91 | 91 | 91 | 91 | 93 | 87 | 90 | 91 | 87 | 90 | 84 | 86 |
| 39 | | | | | | | | | | | | | | | | | | | | | | | |
| Arath_WRKY2 | | | | | | | | | | | | | | | | | | | | | | | |
| 2. SEQ ID NO: | 95 | | 70 | 82 | 87 | 82 | 88 | 83 | 91 | 87 | 86 | 87 | 93 | 87 | 88 | 87 | 87 | 87 | 91 | 87 | 86 | 86 | 86 |
| 39 | | | | | | | | | | | | | | | | | | | | | | | |
| Arath_WRKY20 | | | | | | | | | | | | | | | | | | | | | | | |
| 3. SEQ ID NO: | 88 | 90 | | 75 | 75 | 71 | 76 | 74 | 70 | 76 | 75 | 76 | 70 | 76 | 76 | 71 | 76 | 76 | 70 | 76 | 76 | 76 | 76 |
| 39 | | | | | | | | | | | | | | | | | | | | | | | |
| Arath_WRKY25 | | | | | | | | | | | | | | | | | | | | | | | |
| 4. SEQ ID NO: | 90 | 90 | 86 | | 83 | 83 | 84 | 86 | 86 | 86 | 88 | 86 | 83 | 86 | 83 | 83 | 80 | 83 | 86 | 80 | 86 | 79 | 80 |
| 39 | | | | | | | | | | | | | | | | | | | | | | | |
| Arath_WRKY26 | | | | | | | | | | | | | | | | | | | | | | | |
| 5. SEQ ID NO: | 96 | 95 | 88 | 90 | | 83 | 95 | 92 | 87 | 93 | 92 | 96 | 87 | 96 | 95 | 86 | 93 | 93 | 87 | 93 | 95 | 91 | 92 |
| 39 | | | | | | | | | | | | | | | | | | | | | | | |
| Arath_WRKY33 | | | | | | | | | | | | | | | | | | | | | | | |
| 6. SEQ ID NO: | 91 | 90 | 83 | 87 | 88 | | 84 | 84 | 83 | 86 | 86 | 83 | 82 | 83 | 83 | 83 | 80 | 83 | 83 | 80 | 82 | 79 | 79 |
| 39 | | | | | | | | | | | | | | | | | | | | | | | |
| Arath_WRKY34 | | | | | | | | | | | | | | | | | | | | | | | |
| 7. SEQ ID NO: | 97 | 95 | 88 | 90 | 97 | 88 | | 92 | 88 | 95 | 93 | 96 | 88 | 96 | 96 | 91 | 92 | 95 | 88 | 92 | 95 | 90 | 91 |
| 39 Glyma | | | | | | | | | | | | | | | | | | | | | | | |
| WRKY 2X | | | | | | | | | | | | | | | | | | | | | | | |
| 8. SEQ ID NO: | 97 | 93 | 88 | 91 | 97 | 91 | 95 | | 87 | 93 | 95 | 93 | 86 | 93 | 91 | 88 | 88 | 91 | 86 | 88 | 92 | 86 | 87 |
| 39 Helan | | | | | | | | | | | | | | | | | | | | | | | |
| WRKY 2X | | | | | | | | | | | | | | | | | | | | | | | |
| 9. SEQ ID NO: | 95 | 99 | 90 | 92 | 96 | 88 | 95 | 93 | | 91 | 91 | 88 | 95 | 88 | 88 | 92 | 86 | 87 | 99 | 86 | 87 | 83 | 84 |
| 39 Horvu | | | | | | | | | | | | | | | | | | | | | | | |
| WRKY 2X | | | | | | | | | | | | | | | | | | | | | | | |
| 10. SEQ ID | 97 | 96 | 90 | 90 | 99 | 90 | 97 | 96 | 96 | | 97 | 93 | 90 | 93 | 93 | 88 | 91 | 92 | 90 | 91 | 92 | 88 | 90 |
| NO: 39 Ipoba | | | | | | | | | | | | | | | | | | | | | | | |
| WRKY 2X | | | | | | | | | | | | | | | | | | | | | | | |
| 11. SEQ ID | 96 | 95 | 88 | 92 | 97 | 90 | 96 | 97 | 96 | 97 | | 93 | 88 | 93 | 92 | 87 | 90 | 92 | 90 | 90 | 92 | 87 | 88 |
| NO: 39 | | | | | | | | | | | | | | | | | | | | | | | |
| Lyces_WRKY | | | | | | | | | | | | | | | | | | | | | | | |
| 2X I | | | | | | | | | | | | | | | | | | | | | | | |
| 12. SEQ ID | 96 | 95 | 90 | 92 | 97 | 88 | 97 | 95 | 96 | 97 | 97 | | 87 | 100 | 95 | 87 | 92 | 93 | 88 | 92 | 99 | 90 | 91 |
| NO: 39 | | | | | | | | | | | | | | | | | | | | | | | |
| Lyces_WRKY | | | | | | | | | | | | | | | | | | | | | | | |
| 2X II | | | | | | | | | | | | | | | | | | | | | | | |
| 13. SEQ ID | 93 | 97 | 90 | 90 | 95 | 87 | 96 | 93 | 99 | 96 | 95 | 95 | | 87 | 90 | 90 | 86 | 88 | 96 | 86 | 86 | 83 | 86 |
| NO: 39 | | | | | | | | | | | | | | | | | | | | | | | |
| LycesWRKY | | | | | | | | | | | | | | | | | | | | | | | |
| 2X III | | | | | | | | | | | | | | | | | | | | | | | |
| 14. SEQ ID | 96 | 95 | 90 | 92 | 97 | 88 | 97 | 95 | 96 | 97 | 97 | 100 | 95 | | 95 | 87 | 92 | 93 | 88 | 92 | 99 | 90 | 91 |
| NO: 39 Nicta | | | | | | | | | | | | | | | | | | | | | | | |
| WRKY 2X | | | | | | | | | | | | | | | | | | | | | | | |
| 15. SEQ ID | 95 | 96 | 90 | 88 | 97 | 87 | 96 | 95 | 97 | 97 | 96 | 96 | 97 | 96 | | 87 | 93 | 99 | 88 | 93 | 93 | 91 | 93 |
| NO: 39 | | | | | | | | | | | | | | | | | | | | | | | |
| Orysa_WRKY24 | | | | | | | | | | | | | | | | | | | | | | | |
| 16. SEQ ID | 99 | 93 | 88 | 91 | 96 | 90 | 99 | 96 | 93 | 96 | 95 | 96 | 95 | 96 | 95 | | 83 | 86 | 92 | 83 | 86 | 80 | 82 |
| NO: 39 | | | | | | | | | | | | | | | | | | | | | | | |
| Orysa_WRKY30 | | | | | | | | | | | | | | | | | | | | | | | |
| 17. SEQ ID | 93 | 96 | 90 | 88 | 97 | 87 | 95 | 95 | 97 | 96 | 95 | 95 | 96 | 95 | 97 | 93 | | 92 | 86 | 100 | 91 | 96 | 96 |
| NO: 39 | | | | | | | | | | | | | | | | | | | | | | | |
| Orysa_WRKY53 | | | | | | | | | | | | | | | | | | | | | | | |
| 18. SEQ ID | 95 | 96 | 90 | 88 | 97 | 88 | 96 | 95 | 97 | 97 | 96 | 96 | 97 | 96 | 100 | 95 | 97 | | 87 | 92 | 92 | 90 | 92 |
| NO: 39 | | | | | | | | | | | | | | | | | | | | | | | |
| Orysa_WRKY70 | | | | | | | | | | | | | | | | | | | | | | | |
| 19. SEQ ID | 93 | 97 | 90 | 92 | 95 | 87 | 96 | 93 | 100 | 96 | 96 | 96 | 99 | 96 | 96 | 95 | 96 | 96 | | 86 | 87 | 83 | 84 |
| NO: 39 | | | | | | | | | | | | | | | | | | | | | | | |
| Orysa_WRKY78 | | | | | | | | | | | | | | | | | | | | | | | |
| 20. SEQ ID | 93 | 96 | 90 | 88 | 97 | 87 | 95 | 95 | 97 | 96 | 95 | 95 | 96 | 95 | 97 | 93 | 100 | 97 | 96 | | 91 | 96 | 96 |
| NO: 39 Sacof | | | | | | | | | | | | | | | | | | | | | | | |
| WRKY 2X | | | | | | | | | | | | | | | | | | | | | | | |
| 21. SEQ ID | 95 | 93 | 88 | 91 | 96 | 87 | 96 | 93 | 95 | 96 | 96 | 99 | 93 | 99 | 95 | 95 | 93 | 95 | 95 | 93 | | 88 | 90 |
| NO: 39 Solch | | | | | | | | | | | | | | | | | | | | | | | |
| WRKY 2X | | | | | | | | | | | | | | | | | | | | | | | |
| 22. SEQ ID | 93 | 95 | 90 | 88 | 97 | 88 | 95 | 95 | 97 | 96 | 95 | 95 | 96 | 95 | 97 | 93 | 99 | 97 | 96 | 99 | 93 | | 93 |
| NO: 39 Triae | | | | | | | | | | | | | | | | | | | | | | | |
| WRKY 2X | | | | | | | | | | | | | | | | | | | | | | | |
| 23. SEQ ID | 92 | 95 | 88 | 88 | 95 | 86 | 93 | 92 | 96 | 95 | 93 | 93 | 96 | 93 | 97 | 92 | 96 | 97 | 95 | 96 | 92 | 96 | |
| NO: 39 Zeama | | | | | | | | | | | | | | | | | | | | | | | |
| WRKY 2X | | | | | | | | | | | | | | | | | | | | | | | |

1. A method for increasing plant yield relative to control plants, comprising modulating expression in a plant of a nucleic acid encoding a polypeptide having two WRKY domains or a homologue of such polypeptide, and optionally selecting for plants having increased yield, wherein said polypeptide having two WRKY domains or homologue comprises from amino-terminus to carboxy-terminus: (i) a Pro-Ser rich domain; and (ii) two WRKY domains including a zinc-finger C₂—H₂ motif.

2. The method according to claim 1, wherein said polypeptide having two WRKY domains or homologue further comprises one or more of the following: (i) an acidic stretch between the two WRKY domains where at least 3 out of 6 amino acids are either Asp (D) or Glu (E); (ii) a putative NLS between the two WRKY domains where at least 3 out of 4 amino acids are either Lys (K) or Arg (R); or (iii) a conserved domain with at least 70% identity to SEQ ID NO: 39.

3. The method according to claim 1, wherein said polypeptide having two WRKY domains or homologue further comprises an LXSP motif within said Pro-Ser rich domain wherein L is Leu, S is Ser, P is Pro and X is any amino acid.

4. The method according to claim 1, wherein said Pro-Ser rich domain is at least twice as rich in Pro and Ser compared to an average amino acid composition (in %) of Swiss-Prot Protein Sequence data bank proteins.

5. The method according to claim 1, wherein said modulated expression is effected by introducing a genetic modification in the locus of a gene encoding a polypeptide having two WRKY domains or a homologue of such polypeptide.

6. The method according to claim 5, wherein said genetic modification is effected by one of: T-DNA activation, TILLING, homologus recombination, site-directed mutagenesis or directed evolution.

7. The method according to claim 1, comprising introducing and expressing in the plant the two-WRKY domain nucleic acid or a variant thereof.

8. The method according to claim 7, wherein said variant is a portion of a two-WRKY domain nucleic acid or a sequence capable of hybridizing to a two-WRKY domain nucleic acid, which portion or hybridizing sequence encodes a polypeptide comprising from amino-terminus to carboxy-terminus: (i) a Pro-Ser rich domain; and (ii) two WRKY domains including a zinc-finger C₂—H₂ motif.

9. The method according to claim 7, wherein said two-WRKY domain nucleic acid or variant thereof is overexpressed in a plant.

10. The method according to claim 7, wherein said two-WRKY domain nucleic acid or variant thereof is of plant origin.

11. The method according to claim 7, wherein said variant encodes an orthologue or paralogue of the polypeptide represented by SEQ ID NO: 2 or SEQ ID NO: 51.

12. The method according to claim 7, wherein said two-WRKY domain nucleic acid or variant thereof is operably linked to a constitutive promoter.

13. The method according to claim 12, wherein said constitutive promoter is a GOS2 promoter.

14. The method according to claim 7, wherein said two-WRKY domain nucleic acid or variant thereof is operably linked to an embryo and/or aleurone specific promoter.

15. The method according to claim 14, wherein said embryo and/or aleurone specific promoter is an oleosin promoter.

16. The method according to claim 1, wherein said increased yield is increased seed yield.

17. The method according to claim 1, wherein said increased yield is selected from one or more of the following: increased TKW, increased individual seed area, increased individual seed length, increased individual seed width, increased number of seeds, increased number of flowers per panicle, each relative to control plants.

18. A plant, plant part or plant cell obtained by the method according claim 1.

19. An isolated nucleic acid molecule comprising a nucleic acid molecule selected from the group consisting of:
a) an isolated nucleic acid molecule as depicted in SEQ ID NO: 50;
b) an isolated nucleic acid molecule encoding the amino acid sequence as depicted in SEQ ID NO: 51;
c) an isolated nucleic acid molecule whose sequence can be deduced from a polypeptide sequence as depicted in SEQ ID NO: 51 as a result of the degeneracy of the genetic code;
d) an isolated nucleic acid molecule which encodes a polypeptide which has at least 80% identity with the amino acid sequence of the polypeptide encoded by the nucleic acid molecule of (a) to (c);
e) an isolated nucleic acid molecule encoding a homologue, derivative or active fragment of the amino acid molecule as depicted in SEQ ID NO: 51, which homologue, derivative or fragment is of plant origin and comprises advantageously
(i) an acidic stretch between the two WRKY domains where at least 3 out of 6 amino acids are either Asp (D) or Glu (E);
(ii) a putative NLS between the two WRKY domains where at least 3 out of 4 amino acids are either Lys (K) or Arg (R); and
(iii) a conserved domain with at least 70% identity to SEQ ID NO: 39;
f) an isolated nucleic acid molecule capable of hybridizing with a nucleic acid of (a) to (c) above, or its complement, wherein the hybridizing sequence or the complement thereof encodes the plant protein of (a) to (e);
whereby the nucleic acid molecule has yield and/or growth increasing activities in plants.

20. A construct comprising:
(i) a two-WRKY domain nucleic acid or variant thereof, wherein a polypeptide having two WRKY domains encoded by said nucleic acid or a homologue of said polypeptide comprises from amino-terminus to carboxy-terminus: (i) a Pro-Ser rich domain; and (ii) two WRKY domains including a zinc-finger C₂—H₂ motif;
(ii) one or more control sequences capable of driving expression of the nucleic acid sequence of (i); and optionally
(iii) a transcription termination sequence; or
(iv) a nucleic acid sequence as claimed in claim 19.

21. The construct according to claim 20, wherein said control sequence is a constitutive promoter.

22. The construct according to claim 21, wherein said constitutive promoter is a GOS2 promoter.

23. The construct according to claim 22, wherein said GOS2 promoter is as represented by SEQ ID NO: 42.

24. The construct according to claim 20, wherein said control sequence is an embryo and/or aleurone specific promoter.

25. The construct according to claim 24, wherein said embryo and/or aleurone specific promoter is an oleosin promoter.

26. The construct according to claim 25, wherein said oleosin promoter is as represented by SEQ ID NO: 43.

27. A plant, plant part or plant cell transformed with the nucleic acid sequence as claimed in claim 19.

28. A method for the production of a transgenic plant having increased yield relative to control plants, which method comprises:
(i) introducing and expressing in a plant or plant cell a two-WRKY domain nucleic acid as defined in claim 1 or variant thereof;
(ii) cultivating the plant cell under conditions promoting plant growth and development.

29. A transgenic plant having increased yield resulting from a two-WRKY domain nucleic acid or a variant thereof introduced into said plant.

30. A plant according to claim 18, 27 or 29, wherein said plant is a monocotyledonous plant.

31. Harvestable parts of the plant according to claim 18.

32. Harvestable parts of a plant according to claim 31 wherein said harvestable parts are seeds.

33. Products directly derived from the plant according to claim 30 and/or from harvestable parts therefrom.

34-35. (canceled)

36. A method of selecting a plant with increased plant yield relative to a corresponding control plant, comprising utilizing a two-WRKY domain nucleic acid/gene as defined in claim 1 or variant thereof, or utilizing a polypeptide having two WRKY domains as defined in claim 1 or homologue of such polypeptide, as a molecular marker.

37. The method of claim 7, wherein said two-WRKY domain nucleic acid or variant thereof is from a monocotyledonous plant.

38. The method of claim 7, wherein said two-WRKY domain nucleic acid or variant thereof is from the family Poaceae.

39. The method of claim 7, wherein said two-WRKY domain nucleic acid or variant thereof is from Oryza sativa or Zea mays.

40. A plant, plant part or plant cell transformed with the construct according to claim 20.

41. The plant according to claim 18, wherein said plant is sugar cane, rice, maize, wheat, barley, millet, rye, oats, or sorghum.

42. The method of claim 2, wherein the conserved domain has at least 95% identity to SEQ ID NO: 39.
